(19) United States
(12) Patent Application Publication
(10) Pub. No.: US 2023/0027978 A1
(43) Pub. Date: Jan. 26, 2023

(54) MACHINE-LEARNED MODELS IN SUPPORT OF SURGICAL PROCEDURES

(71) Applicant: Howmedica Osteonics Corp., Mahwah, NJ (US)

(72) Inventors: Vincent Gaborit, Saint Martin D?Hères (FR); Karine Mollard, Le Versoud (FR); Marine Mayer, Grenoble (FR); François Boux de Casson, Le Sappey en Chartreuse (FR); Manuel Jean-Marie Urvoy, Brest (FR)

(21) Appl. No.: 17/780,445

(22) PCT Filed: Nov. 30, 2020
(86) PCT No.: PCT/US2020/062567

§ 371 Date: May 26, 2022

Related U.S. Application Data
(60) Provisional application No. 62/942,956, filed on Dec. 3, 2019.

(51) Int. Cl. A61B 34/10

(52) U.S. Cl. CPC A61B 34/10; A61B 2017/00017

(57) ABSTRACT

The disclosure describes examples of machine-learned model based techniques. A computing system may obtain patient characteristics of a patient and implant characteristics of an implant. The computing system may determine information indicative of an operational duration of the implant based on the patient characteristics and the implant characteristics and output the information indicative of the operational duration of the implant. In some examples, one or more processors may be configured to receive, with a machine-learned model of the computing system, implant characteristics of an implant to be manufactured, apply model parameters of the machine-learned model to the implant characteristics, determine information indicative of dimensions of the implant to be manufactured based on the applying of the model parameters of the machine-learned model, and output the information indicative of the dimensions of the implant to be manufactured.

This application claims the benefit of U.S. Patent Application No. 62/942,956 filed on Dec. 3, 2019, the entire content of which is incorporated herein by reference.

### BACKGROUND

Surgical joint repair procedures involve repair and/or replacement of a damaged or diseased joint. Many times, a surgical joint repair procedure, such as joint arthroplasty as an example, involves replacing the damaged joint with a prosthetic that is implanted into the patient's bone. Proper selection of a prosthetic that is appropriately sized and shaped and proper positioning of that prosthetic to ensure an optimal surgical outcome can be challenging. Various tools may assist surgeons with preoperative planning for joint repairs and replacements.

### SUMMARY

This disclosure describes a variety of techniques for providing preoperative planning, medical implant design and manufacture, intraoperative guidance, postoperative analysis, and/or training and education for surgical joint repair procedures. The techniques may be used independently or in various combinations to support particular phases or settings for surgical joint repair procedures or provide a multi-faceted ecosystem to support surgical joint repair procedures. In various examples, the disclosure describes techniques for preoperative surgical planning, intra-operative surgical planning, intra-operative surgical guidance, intra-operative surgical tracking and post-operative analysis using mixed reality (MR)-based visualization. In some examples, the disclosure also describes surgical items and/or methods for performing surgical joint repair procedures. In some examples, this disclosure also describes techniques and visualization devices configured to provide education about an orthopedic surgical procedure using mixed reality.

This disclosure describes a variety of techniques for using machine learning to determine operational duration of an orthopedic implant in a pre-operative or intraoperative setting. A computing system may determine the operational duration of the implant such as an estimate of how long an implant will effectively serve its intended function after implantation before subsequent action, e.g., additional surgery such as a revision procedure, is needed. A revision procedure may involve replacement of an orthopedic implant with a new implant. For example, the computing system may configure a machine-learned model with a machine learning dataset that includes information used to predict the operational duration of the orthopedic implant. The machine-learned model may receive patient and implant characteristics and use the model parameters of the machine-learned model generated from the machine learning dataset to determine information indicative of the predicted operational duration of the implant.

In this manner, a surgeon can receive information indicative of an estimate of the operational duration of a particular implant. A longer operational duration is ordinarily desirable so as to prolong effective operation and delay the need for a surgical revision procedure. The surgeon can then determine whether the particular implant is a suitable implant for the patient or whether a different implant is more suitable, e.g., based on prediction of a longer operational duration. In some examples, the computing system may determine the operational duration of multiple implants and provide a recommendation to the surgeon based on the operational duration of the multiple implants, and in some examples, accounting for patient characteristics.

Accordingly, the example techniques rely on computational processes rooted in machine learning technologies as a way to provide a practical application of selecting an implant for implantation. The techniques described in this disclosure may allow a surgeon to select the suitable implant based on more than just know-how and experience of the surgeon, which may be especially limited for less experienced surgeons.

In one example, the disclosure describes a computer-implemented method comprising obtaining, by a computing system, patient characteristics of a patient, obtaining, by the computing system, implant characteristics of an implant, determining, by the computing system, information indicative of an operational duration of the implant based on the patient characteristics and the implant characteristics, and outputting, by the computing system, the information indicative of the operational duration of the implant.

In one example, the disclosure describes a computing system comprising memory configured to store patient characteristics of a patient and implant characteristics of an implant and one or more processors, coupled to the memory, and configured to obtain the patient characteristics of the patient, obtain the implant characteristics of the implant, determine information indicative of an operational duration of the implant based on the patient characteristics and the implant characteristics, and output the information indicative of the operational duration of the implant.

In one example, the disclosure describes a computer-readable storage medium storing instructions thereon that when executed cause one or more processors to obtain patient characteristics of a patient, obtain implant characteristics of an implant, determine information indicative of an operational duration of the implant based on the patient characteristics and the implant characteristics, and output the information indicative of the operational duration of the implant.

In one example, the disclosure describes a computer system comprising means for obtaining patient characteristics of a patient, means for obtaining implant characteristics of an implant, means for determining information indicative of an operational duration of the implant based on the patient characteristics and the implant characteristics, and means for outputting the information indicative of the operational duration of the implant.

This disclosure describes a variety of techniques for using machine learning to determine information indicative of dimensions of an orthopedic implant based on implant characteristics. For instance, a machine-learned model may receive the implant characteristics such as information that the implant is used for a type of surgery (e.g., reverse or anatomical shoulder replacement surgery), information that the implant is stemmed or stemless, information that the implant is for a type of patient condition (e.g., fracture, cuff tear, or osteoarthritis), and information that the implant is for a particular bone (e.g., humerus or glenoid). The machine-learned model may apply model parameters of the machine-learned model, where the model parameters are generated from a machine learning data set, and determine information indicative of the dimensions based on the applying of the model parameters of the machine-learned model. A manufacturer may then construct the implant based on the determined dimensions.

In one or more examples, the determination of the information indicative of the dimensions of the implant may be applicable to many patients rather than determined for a specific patient. In other words, the machine-learned model may determine the information indicative of the dimensions of the implant without relying on patient specific information such that the resulting implant having the dimensions may be suitable for many patients.

Accordingly, the example techniques may rely on the computational processes rooted in machine learning technologies as a way to provide a practical application of determining dimensions of an implant for designing and constructing the implant. The techniques described in this disclosure allow an implant designer to design an implant relying on more than know-how and experience of the implant designer, which may be especially limited for less experienced designers.

In one example, the disclosure describes a computer-implemented method comprising receiving, with a machine-learned model of the computing system, implant characteristics of an implant to be manufactured, applying, with the computing system, model parameters of the machine-learned model to the implant characteristics, wherein the model parameters of the machine-learned model are generated based on a machine learning dataset, determining information indicative of dimensions of the implant to be manufactured based on the applying of the model parameters of the machine-learned model, and outputting, by the computing system, the information indicative of the dimensions of the implant to be manufactured.

In one example, the disclosure describes a computing system comprising memory configured to store implant characteristics of an implant to be manufactured and one or more processors configured to receive, with a machine-learned model of the computing system, the implant characteristics of the implant to be manufactured, apply model parameters of the machine-learned model to the implant characteristics, wherein the model parameters of the machine-learned model are generated based on a machine learning dataset, determine information indicative of dimensions of the implant to be manufactured based on the applying of the model parameters of the machine-learned model, and output the information indicative of the dimensions of the implant to be manufactured.

In one example, the disclosure describes a computer-readable storage medium storing instructions thereon that when executed cause one or more processors to receive implant characteristics of an implant to be manufactured, apply model parameters of the machine-learned model to the implant characteristics, wherein the model parameters of the machine-learned model are generated based on a machine learning dataset, determine information indicative of dimensions of the implant to be manufactured based on the applying of the model parameters of the machine-learned model, and output the information indicative of the dimensions of the implant to be manufactured.

In one example, the disclosure describes a computer system comprising means for receiving implant characteristics of an implant to be manufactured, means for applying model parameters of the machine-learned model to the implant characteristics, wherein the model parameters of the machine-learned model are generated based on a machine learning dataset, means for determining information indicative of dimensions of the implant to be manufactured based on the applying of the model parameters of the machine-learned model, and means for outputting, by the computing system, the information indicative of the dimensions of the implant to be manufactured.

The details of various examples of the disclosure are set forth in the accompanying drawings and the description below. Various features, objects, and advantages will be apparent from the description, drawings, and claims.

### BRIEF DESCRIPTION OF THE DRAWINGS

FIG. 1 is a block diagram of an orthopedic surgical system according to an example of this disclosure.

FIG. 2 is a block diagram of an orthopedic surgical system that includes a mixed reality (MR) system, according to an example of this disclosure.

FIG. 3 is a flowchart illustrating example phases of a surgical lifecycle.

FIG. 4 is a flowchart illustrating preoperative, intraoperative and postoperative workflows in support of an orthopedic surgical procedure.

FIG. 5 is a schematic representation of a visualization device for use in a mixed reality (MR) system, according to an example of this disclosure.

FIG. 6 is a block diagram illustrating example components of a visualization device for use in a mixed reality (MR) system, according to an example of this disclosure.

FIG. 7 is a block diagram illustrating example components of a virtual planning system, according to an example of this disclosure.

FIG. 8 is a flowchart illustrating example steps in the preoperative phase of the surgical lifecycle.

FIGS. 9 through 13 are conceptual diagrams illustrating aspects of an example machine-learned model according to example implementations of the present disclosure.

FIG. 14 is a flowchart illustrating an example method of determining information indicative of an operational duration of an implant.

FIG. 15 is a flowchart illustrating example method of selecting an implant.

FIG. 16 is a flowchart illustrating another example method of selecting an implant.

FIG. 17 is a flowchart illustrating an example method of determining information indicative of dimensions of an implant.

FIG. 18 is a flowchart illustrating an example operation in accordance with one or more techniques of this disclosure.

FIG. 19 is a flowchart illustrating an example operation of a virtual planning system to determine an estimated operating room time for a surgical procedure to be performed on a patient, in accordance with one or more techniques of this disclosure.

### DETAILED DESCRIPTION

Certain examples of this disclosure are described with reference to the accompanying drawings, wherein like reference numerals denote like elements. It should be understood, however, that the accompanying drawings illustrate only the various implementations described herein and are not meant to limit the scope of various technologies described herein. The drawings show and describe various examples of this disclosure.

In the following description, numerous details are set forth to provide an understanding of the present disclosure. However, it will be understood by those skilled in the art that the present disclosure may be practiced without these details and that numerous variations or modifications from the described examples may be possible.

Orthopedic surgery can involve implanting one or more prosthetic devices to repair or replace a patient's damaged or diseased joint. Today, virtual surgical planning tools are available that use image data of the diseased or damaged joint to generate an accurate three-dimensional bone model that can be viewed and manipulated preoperatively by the surgeon. These tools can enhance surgical outcomes by allowing the surgeon to simulate the surgery, select or design an implant that more closely matches the contours of the patient's actual bone, and select or design surgical instruments and guide tools that are adapted specifically for repairing the bone of a particular patient. Use of these planning tools typically results in generation of a preoperative surgical plan, complete with an implant and surgical instruments that are selected or manufactured for the individual patient. Oftentimes, once in the actual operating environment, the surgeon may desire to verify the preoperative surgical plan intraoperatively relative to the patient's actual bone. This verification may result in a determination that an adjustment to the preoperative surgical plan is needed, such as a different implant, a different positioning or orientation of the implant, and/or a different surgical guide for carrying out the surgical plan. In addition, a surgeon may want to view details of the preoperative surgical plan relative to the patient's real bone during the actual procedure in order to more efficiently and accurately position and orient the implant components. For example, the surgeon may want to obtain intra-operative visualization that provides guidance for positioning and orientation of implant components, guidance for preparation of bone or tissue to receive the implant components, guidance for reviewing the details of a procedure or procedural step, and/or guidance for selection of tools or implants and tracking of surgical procedure workflow.

Accordingly, this disclosure describes systems and methods for using a mixed reality (MR) visualization system to assist with creation, implementation, verification, and/or modification of a surgical plan before and during a surgical procedure. Because MR, or in some instances VR, may be used to interact with the surgical plan, this disclosure may also refer to the surgical plan as a “virtual” surgical plan. Visualization tools other than or in addition to mixed reality visualization systems may be used in accordance with techniques of this disclosure. A surgical plan, e.g., as generated by the BLUEPRINT™ system or another surgical planning platform, may include information defining a variety of features of a surgical procedure, such as features of particular surgical procedure steps to be performed on a patient by a surgeon according to the surgical plan including, for example, bone or tissue preparation steps and/or steps for selection, modification and/or placement of implant components. Such information may include, in various examples, dimensions, shapes, angles, surface contours, and/or orientations of implant components to be selected or modified by surgeons, dimensions, shapes, angles, surface contours and/or orientations to be defined in bone or tissue by the surgeon in bone or tissue preparation steps, and/or positions, axes, planes, angle and/or entry points defining placement of implant components by the surgeon relative to patient bone or tissue. Information such as dimensions, shapes, angles, surface contours, and/or orientations of anatomical features of the patient may be derived from imaging (e.g., x-ray, CT, MRI, ultrasound or other images), direct observation, or other techniques.

In this disclosure, the term “mixed reality” (MR) refers to the presentation of virtual objects such that a user sees images that include both real, physical objects and virtual objects. Virtual objects may include text, 2-dimensional surfaces, 3-dimensional models, or other user-perceptible elements that are not actually present in the physical, real-world environment in which they are presented as coexisting. In addition, virtual objects described in various examples of this disclosure may include graphics, images, animations or videos, e.g., presented as 3D virtual objects or 2D virtual objects. Virtual objects may also be referred to as virtual elements. Such elements may or may not be analogs of real-world objects. In some examples, in mixed reality, a camera may capture images of the real world and modify the images to present virtual objects in the context of the real world. In such examples, the modified images may be displayed on a screen, which may be head-mounted, handheld, or otherwise viewable by a user. This type of mixed reality is increasingly common on smartphones, such as where a user can point a smartphone's camera at a sign written in a foreign language and see in the smartphone's screen a translation in the user's own language of the sign superimposed on the sign along with the rest of the scene captured by the camera. In some examples, in mixed reality, see-through (e.g., transparent) holographic lenses, which may be referred to as waveguides, may permit the user to view real-world objects, i.e., actual objects in a real-world environment, such as real anatomy, through the holographic lenses and also concurrently view virtual objects.

The Microsoft HOLOLENS™ headset, available from Microsoft Corporation of Redmond, Washington, is an example of a MR device that includes see-through holographic lenses, sometimes referred to as waveguides, that permit a user to view real-world objects through the lens and concurrently view projected 3D holographic objects. The Microsoft HOLOLENS™ headset, or similar waveguide-based visualization devices, are examples of an MR visualization device that may be used in accordance with some examples of this disclosure. Some holographic lenses may present holographic objects with some degree of transparency through see-through holographic lenses so that the user views real-world objects and virtual, holographic objects. In some examples, some holographic lenses may, at times, completely prevent the user from viewing real-world objects and instead may allow the user to view entirely virtual environments. The term mixed reality may also encompass scenarios where one or more users are able to perceive one or more virtual objects generated by holographic projection. In other words, “mixed reality” may encompass the case where a holographic projector generates holograms of elements that appear to a user to be present in the user's actual physical environment.

In some examples, in mixed reality, the positions of some or all presented virtual objects are related to positions of physical objects in the real world. For example, a virtual object may be tethered to a table in the real world, such that the user can see the virtual object when the user looks in the direction of the table but does not see the virtual object when the table is not in the user's field of view. In some examples, in mixed reality, the positions of some or all presented virtual objects are unrelated to positions of physical objects in the real world. For instance, a virtual item may always appear in the top right of the user's field of vision, regardless of where the user is looking.

Augmented reality (AR) is similar to MR in the presentation of both real-world and virtual elements, but AR generally refers to presentations that are mostly real, with a few virtual additions to “augment” the real-world presentation. For purposes of this disclosure, MR is considered to include AR. For example, in AR, parts of the user's physical environment that are in shadow can be selectively brightened without brightening other areas of the user's physical environment. This example is also an instance of MR in that the selectively brightened areas may be considered virtual objects superimposed on the parts of the user's physical environment that are in shadow.

Furthermore, in this disclosure, the term “virtual reality” (VR) refers to an immersive artificial environment that a user experiences through sensory stimuli (such as sights and sounds) provided by a computer. Thus, in virtual reality, the user may not see any physical objects as they exist in the real world. Video games set in imaginary worlds are a common example of VR. The term “VR” also encompasses scenarios where the user is presented with a fully artificial environment in which some virtual object's locations are based on the locations of corresponding physical objects as they relate to the user. Walk-through VR attractions are examples of this type of VR.

The term “extended reality” (XR) is a term that encompasses a spectrum of user experiences that includes virtual reality, mixed reality, augmented reality, and other user experiences that involve the presentation of at least some perceptible elements as existing in the user's environment that are not present in the user's real-world environment. Thus, the term “extended reality” may be considered a genus for MR and VR. XR visualizations may be presented in any of the techniques for presenting mixed reality discussed elsewhere in this disclosure or presented using techniques for presenting VR, such as VR goggles.

These mixed reality systems and methods can be part of an intelligent surgical planning system that includes multiple subsystems that can be used to enhance surgical outcomes. In addition to the preoperative and intraoperative applications discussed above, an intelligent surgical planning system can include postoperative tools to assist with patient recovery and which can provide information that can be used to assist with and plan future surgical revisions or surgical cases for other patients.

Accordingly, systems and methods are also described herein that can be incorporated into an intelligent surgical planning system, such as artificial intelligence systems to assist with planning, implants with embedded sensors (e.g., smart implants) to provide postoperative feedback for use by the healthcare provider and the artificial intelligence system, and mobile applications to monitor and provide information to the patient and the healthcare provider in real-time or near real-time.

Visualization tools are available that utilize patient image data to generate three-dimensional models of bone contours to facilitate preoperative planning for joint repairs and replacements. These tools allow surgeons to design and/or select surgical guides and implant components that closely match the patient's anatomy. These tools can improve surgical outcomes by customizing a surgical plan for each patient. An example of such a visualization tool for shoulder repairs is the BLUEPRINT™ system available from Wright Medical Technology, Inc. The BLUEPRINT™ system provides the surgeon with two-dimensional planar views of the bone repair region as well as a three-dimensional virtual model of the repair region. The surgeon can use the BLUEPRINT™ system to select, design or modify appropriate implant components, determine how best to position and orient the implant components and how to shape the surface of the bone to receive the components, and design, select or modify surgical guide tool(s) or instruments to carry out the surgical plan. The information generated by the BLUEPRINT™ system is compiled in a preoperative surgical plan for the patient that is stored in a database at an appropriate location (e.g., on a server in a wide area network, a local area network, or a global network) where it can be accessed by the surgeon or other care provider, including before and during the actual surgery.

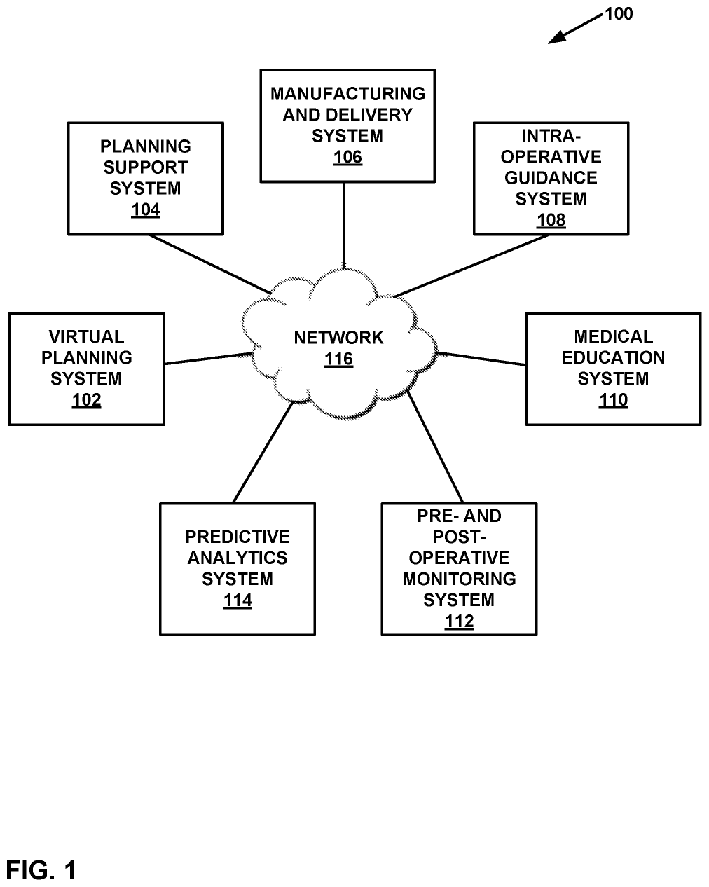

FIG. 1 is a block diagram of an orthopedic surgical system 100 according to an example of this disclosure. Orthopedic surgical system 100 includes a set of subsystems. In the example of FIG. 1, the subsystems include a virtual planning system 102, a planning support system 104, a manufacturing and delivery system 106, an intraoperative guidance system 108, a medical education system 110, a monitoring system 112, a predictive analytics system 114, and a communications network 116. In other examples, orthopedic surgical system 100 may include more, fewer, or different subsystems. For example, orthopedic surgical system 100 may omit medical education system 110, monitoring system 112, predictive analytics system 114, and/or other subsystems. In some examples, orthopedic surgical system 100 may be used for surgical tracking, in which case orthopedic surgical system 100 may be referred to as a surgical tracking system. In other cases, orthopedic surgical system 100 may be generally referred to as a medical device system.

Users of orthopedic surgical system 100 may use virtual planning system 102 to plan orthopedic surgeries. Users of orthopedic surgical system 100 may use planning support system 104 to review surgical plans generated using orthopedic surgical system 100. Manufacturing and delivery system 106 may assist with the manufacture and delivery of items needed to perform orthopedic surgeries. Intraoperative guidance system 108 provides guidance to assist users of orthopedic surgical system 100 in performing orthopedic surgeries. Medical education system 110 may assist with the education of users, such as healthcare professionals, patients, and other types of individuals. Pre- and postoperative monitoring system 112 may assist with monitoring patients before and after the patients undergo surgery. Predictive analytics system 114 may assist healthcare professionals with various types of predictions. For example, predictive analytics system 114 may apply artificial intelligence techniques to determine a classification of a condition of an orthopedic joint, e.g., a diagnosis, determine which type of surgery to perform on a patient and/or which type of implant to be used in the procedure, determine types of items that may be needed during the surgery, and so on.

The subsystems of orthopedic surgical system 100 (i.e., virtual planning system 102, planning support system 104, manufacturing and delivery system 106, intraoperative guidance system 108, medical education system 110, pre- and postoperative monitoring system 112, and predictive analytics system 114) may include various systems. The systems in the subsystems of orthopedic surgical system 100 may include various types of computing systems, computing devices, including server computers, personal computers, tablet computers, smartphones, display devices, Internet of Things (IoT) devices, visualization devices (e.g., mixed reality (MR) visualization devices, virtual reality (VR) visualization devices, holographic projectors, or other devices for presenting extended reality (XR) visualizations), surgical tools, and so on. A holographic projector, in some examples, may project a hologram for general viewing by multiple users or a single user without a headset, rather than viewing only by a user wearing a headset. For example, virtual planning system 102 may include a MR visualization device and one or more server devices, planning support system 104 may include one or more personal computers and one or more server devices, and so on. A computing system is a set of one or more computing systems configured to operate as a system. In some examples, one or more devices may be shared between two or more of the subsystems of orthopedic surgical system 100. For instance, in the previous examples, virtual planning system 102 and planning support system 104 may include the same server devices.

In the example of FIG. 1, the devices included in the subsystems of orthopedic surgical system 100 may communicate using communications network 116. Communications network 116 may include various types of communication networks including one or more wide-area networks, such as the Internet, local area networks, and so on. In some examples, communications network 116 may include wired and/or wireless communication links.

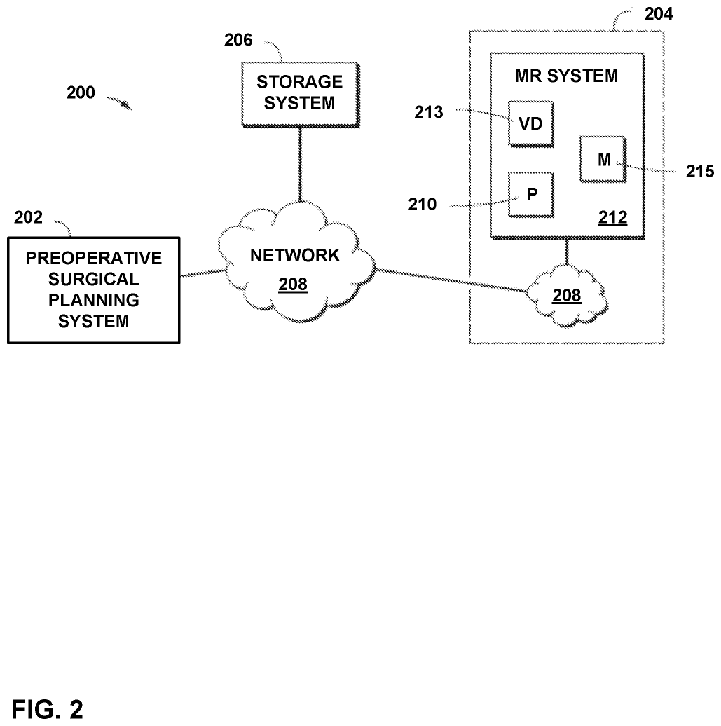

Many variations of orthopedic surgical system 100 are possible in accordance with techniques of this disclosure. Such variations may include more or fewer subsystems than the version of orthopedic surgical system 100 shown in FIG. 1. For example, FIG. 2 is a block diagram of an orthopedic surgical system 200 that includes one or more mixed reality (MR) systems, according to an example of this disclosure. Orthopedic surgical system 200 may be used for creating, verifying, updating, modifying and/or implementing a surgical plan. In some examples, the surgical plan can be created preoperatively, such as by using a virtual surgical planning system (e.g., the BLUEPRINT™ system), and then verified, modified, updated, and viewed intraoperatively, e.g., using MR visualization of the surgical plan. In other examples, orthopedic surgical system 200 can be used to create the surgical plan immediately prior to surgery or intraoperatively, as needed. In some examples, orthopedic surgical system 200 may be used for surgical tracking, in which case orthopedic surgical system 200 may be referred to as a surgical tracking system. In other cases, orthopedic surgical system 200 may be generally referred to as a medical device system.

In the example of FIG. 2, orthopedic surgical system 200 includes a preoperative surgical planning system 202, a healthcare facility 204 (e.g., a surgical center or hospital), a storage system 206, and a network 208 that allows a user at healthcare facility 204 to access stored patient information, such as medical history, image data corresponding to the damaged joint or bone and various parameters corresponding to a surgical plan that has been created preoperatively (as examples). Preoperative surgical planning system 202 may be equivalent to virtual planning system 102 of FIG. 1 and, in some examples, may generally correspond to a virtual planning system similar or identical to the BLUEPRINT™ system.

In the example of FIG. 2, healthcare facility 204 includes a mixed reality (MR) system 212. In some examples of this disclosure, MR system 212 includes one or more processing device(s) (P) 210 to provide functionalities that will be described in further detail below. Processing device(s) 210 may also be referred to as processor(s). In addition, one or more users of MR system 212 (e.g., a surgeon, nurse, or other care provider) can use processing device(s) (P) 210 to generate a request for a particular surgical plan or other patient information that is transmitted to storage system 206 via network 208. In response, storage system 206 returns the requested patient information to MR system 212. In some examples, the users can use other processing device(s) to request and receive information, such as one or more processing devices that are part of MR system 212, but not part of any visualization device, or one or more processing devices that are part of a visualization device (e.g., visualization device 213) of MR system 212, or a combination of one or more processing devices that are part of MR system 212, but not part of any visualization device, and one or more processing devices that are part of a visualization device (e.g., visualization device 213) that is part of MR system 212.

In some examples, multiple users can simultaneously use MR system 212. For example, MR system 212 can be used in a spectator mode in which multiple users each use their own visualization devices so that the users can view the same information at the same time and from the same point of view. In some examples, MR system 212 may be used in a mode in which multiple users each use their own visualization devices so that the users can view the same information from different points of view.

In some examples, processing device(s) 210 can provide a user interface to display data and receive input from users at healthcare facility 204. Processing device(s) 210 may be configured to control visualization device 213 to present a user interface. Furthermore, processing device(s) 210 may be configured to control visualization device 213 to present virtual images, such as 3D virtual models, 2D images, and so on. Processing device(s) 210 can include a variety of different processing or computing devices, such as servers, desktop computers, laptop computers, tablets, mobile phones and other electronic computing devices, or processors within such devices. In some examples, one or more of processing device(s) 210 can be located remote from healthcare facility 204. In some examples, processing device(s) 210 reside within visualization device 213. In some examples, at least one of processing device(s) 210 is external to visualization device 213. In some examples, one or more processing device(s) 210 reside within visualization device 213 and one or more of processing device(s) 210 are external to visualization device 213.

In the example of FIG. 2, MR system 212 also includes one or more memory or storage device(s) (M) 215 for storing data and instructions of software that can be executed by processing device(s) 210. The instructions of software can correspond to the functionality of MR system 212 described herein. In some examples, the functionalities of a virtual surgical planning application, such as the BLUEPRINT™ system, can also be stored and executed by processing device(s) 210 in conjunction with memory storage device(s) (M) 215. For instance, memory or storage system 215 may be configured to store data corresponding to at least a portion of a virtual surgical plan. In some examples, storage system 206 may be configured to store data corresponding to at least a portion of a virtual surgical plan. In some examples, memory or storage device(s) (M) 215 reside within visualization device 213. In some examples, memory or storage device(s) (M) 215 are external to visualization device 213. In some examples, memory or storage device(s) (M) 215 include a combination of one or more memory or storage devices within visualization device 213 and one or more memory or storage devices external to the visualization device.

Network 208 may be equivalent to network 116. Network 208 can include one or more wide area networks, local area networks, and/or global networks (e.g., the Internet) that connect preoperative surgical planning system 202 and MR system 212 to storage system 206. Storage system 206 can include one or more databases that can contain patient information, medical information, patient image data, and parameters that define the surgical plans. For example, medical images of the patient's diseased or damaged bone typically are generated preoperatively in preparation for an orthopedic surgical procedure. The medical images can include images of the relevant bone(s) taken along the sagittal plane and the coronal plane of the patient's body. The medical images can include X-ray images, magnetic resonance imaging (MRI) images, computerized tomography (CT) images, ultrasound images, and/or any other type of 2D or 3D image that provides information about the relevant surgical area. Storage system 206 also can include data identifying the implant components selected for a particular patient (e.g., type, size, etc.), surgical guides selected for a particular patient, and details of the surgical procedure, such as entry points, cutting planes, drilling axes, reaming depths, etc. Storage system 206 can be a cloud-based storage system (as shown) or can be located at healthcare facility 204 or at the location of preoperative surgical planning system 202 or can be part of MR system 212 or visualization device (VD) 213, as examples.

MR system 212 can be used by a surgeon before (e.g., preoperatively) or during the surgical procedure (e.g., intraoperatively) to create, review, verify, update, modify and/or implement a surgical plan. In some examples, MR system 212 may also be used after the surgical procedure (e.g., postoperatively) to review the results of the surgical procedure, assess whether revisions are required, or perform other postoperative tasks. To that end, MR system 212 may include a visualization device 213 that may be worn by the surgeon and (as will be explained in further detail below) is operable to display a variety of types of information, including a 3D virtual image of the patient's diseased, damaged, or postsurgical joint and details of the surgical plan, such as a 3D virtual image of the prosthetic implant components selected for the surgical plan, 3D virtual images of entry points for positioning the prosthetic components, alignment axes and cutting planes for aligning cutting or reaming tools to shape the bone surfaces, or drilling tools to define one or more holes in the bone surfaces, in the surgical procedure to properly orient and position the prosthetic components, surgical guides and instruments and their placement on the damaged joint, and any other information that may be useful to the surgeon to implement the surgical plan. MR system 212 can generate images of this information that are perceptible to the user of the visualization device 213 before and/or during the surgical procedure.

In some examples, MR system 212 includes multiple visualization devices (e.g., multiple instances of visualization device 213) so that multiple users can simultaneously see the same images and share the same 3D scene. In some such examples, one of the visualization devices can be designated as the master device and the other visualization devices can be designated as observers or spectators. Any observer device can be re-designated as the master device at any time, as may be desired by the users of MR system 212.

In this way, FIG. 2 illustrates a surgical planning system that includes a preoperative surgical planning system 202 to generate a virtual surgical plan customized to repair an anatomy of interest of a particular patient. For example, the virtual surgical plan may include a plan for an orthopedic joint repair surgical procedure (e.g., to attach a prosthetic to anatomy of a patient), such as one of a standard total shoulder arthroplasty or a reverse shoulder arthroplasty. In this example, details of the virtual surgical plan may include details relating to at least one of preparation of anatomy for attachment of a prosthetic or attachment of the prosthetic to the anatomy. For instance, details of the virtual surgical plan may include details relating to at least one of preparation of a glenoid bone, preparation of a humeral bone, attachment of a prosthetic to the glenoid bone, or attachment of a prosthetic to the humeral bone. In some examples, the orthopedic joint repair surgical procedure is one of a stemless standard total shoulder arthroplasty, a stemmed standard total shoulder arthroplasty, a stemless reverse shoulder arthroplasty, a stemmed reverse shoulder arthroplasty, an augmented glenoid standard total shoulder arthroplasty, and an augmented glenoid reverse shoulder arthroplasty.

The virtual surgical plan may include a 3D virtual model corresponding to the anatomy of interest of the particular patient and a 3D model of a prosthetic component matched to the particular patient to repair the anatomy of interest or selected to repair the anatomy of interest. Furthermore, in the example of FIG. 2, the surgical planning system includes a storage system 206 to store data corresponding to the virtual surgical plan. The surgical planning system of FIG. 2 also includes MR system 212, which may comprise visualization device 213. In some examples, visualization device 213 is wearable by a user. In some examples, visualization device 213 is held by a user, or rests on a surface in a place accessible to the user. MR system 212 may be configured to present a user interface via visualization device 213. The user interface may present details of the virtual surgical plan for a particular patient. For instance, the details of the virtual surgical plan may include a 3D virtual model of an anatomy of interest of the particular patient. The user interface is visually perceptible to the user when the user is using visualization device 213. For instance, in one example, a screen of visualization device 213 may display real-world images and the user interface on a screen. In some examples, visualization device 213 may project virtual, holographic images onto see-through holographic lenses and also permit a user to see real-world objects of a real-world environment through the lenses. In other words, visualization device 213 may comprise one or more see-through holographic lenses and one or more display devices that present imagery to the user via the holographic lenses to present the user interface to the user.

In some examples, visualization device 213 is configured such that the user can manipulate the user interface (which is visually perceptible to the user when the user is wearing or otherwise using visualization device 213) to request and view details of the virtual surgical plan for the particular patient, including a 3D virtual model of the anatomy of interest (e.g., a 3D virtual bone model of the anatomy of interest, such as a glenoid bone or a humeral bone) and/or a 3D model of the prosthetic component selected to repair an anatomy of interest. In some such examples, visualization device 213 is configured such that the user can manipulate the user interface so that the user can view the virtual surgical plan intraoperatively, including (at least in some examples) the 3D virtual model of the anatomy of interest (e.g., a 3D virtual bone model of the anatomy of interest). In some examples, MR system 212 can be operated in an augmented surgery mode in which the user can manipulate the user interface intraoperatively so that the user can visually perceive details of the virtual surgical plan projected in a real environment, e.g., on a real anatomy of interest of the particular patient. In this disclosure, the terms real and real world may be used in a similar manner. For example, MR system 212 may present one or more virtual objects that provide guidance for preparation of a bone surface and placement of a prosthetic implant on the bone surface. Visualization device 213 may present one or more virtual objects in a manner in which the virtual objects appear to be overlaid on an actual, real anatomical object of the patient, within a real-world environment, e.g., by displaying the virtual object(s) with actual, real-world patient anatomy viewed by the user through holographic lenses. For example, the virtual objects may be 3D virtual objects that appear to reside within the real-world environment with the actual, real anatomical object.

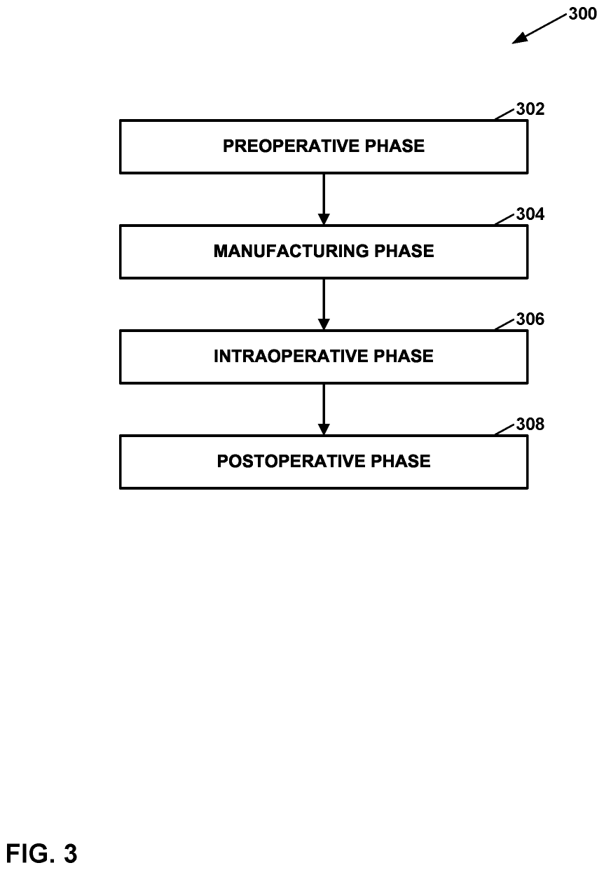

FIG. 3 is a flowchart illustrating example phases of a surgical lifecycle 300. In the example of FIG. 3, surgical lifecycle 300 begins with a preoperative phase (302). During the preoperative phase, a surgical plan is developed. The preoperative phase is followed by a manufacturing and delivery phase (304). During the manufacturing and delivery phase, patient-specific items, such as parts and equipment, needed for executing the surgical plan are manufactured and delivered to a surgical site. In some examples, it is unnecessary to manufacture patient-specific items in order to execute the surgical plan. An intraoperative phase follows the manufacturing and delivery phase (306). The surgical plan is executed during the intraoperative phase. In other words, one or more persons perform the surgery on the patient during the intraoperative phase. The intraoperative phase is followed by the postoperative phase (308). The postoperative phase includes activities occurring after the surgical plan is complete. For example, the patient may be monitored during the postoperative phase for complications.

As described in this disclosure, orthopedic surgical system 100 (FIG. 1) may be used in one or more of preoperative phase 302, the manufacturing and delivery phase 304, the intraoperative phase 306, and the postoperative phase 308. For example, virtual planning system 102 and planning support system 104 may be used in preoperative phase 302. Manufacturing and delivery system 106 may be used in the manufacturing and delivery phase 304. Intraoperative guidance system 108 may be used in intraoperative phase 306. Some of the systems of FIG. 1 may be used in multiple phases of FIG. 3. For example, medical education system 110 may be used in one or more of preoperative phase 302, intraoperative phase 306, and postoperative phase 308; pre- and postoperative monitoring system 112 may be used in preoperative phase 302 and postoperative phase 308. Predictive analytics system 114 may be used in preoperative phase 302 and postoperative phase 308.

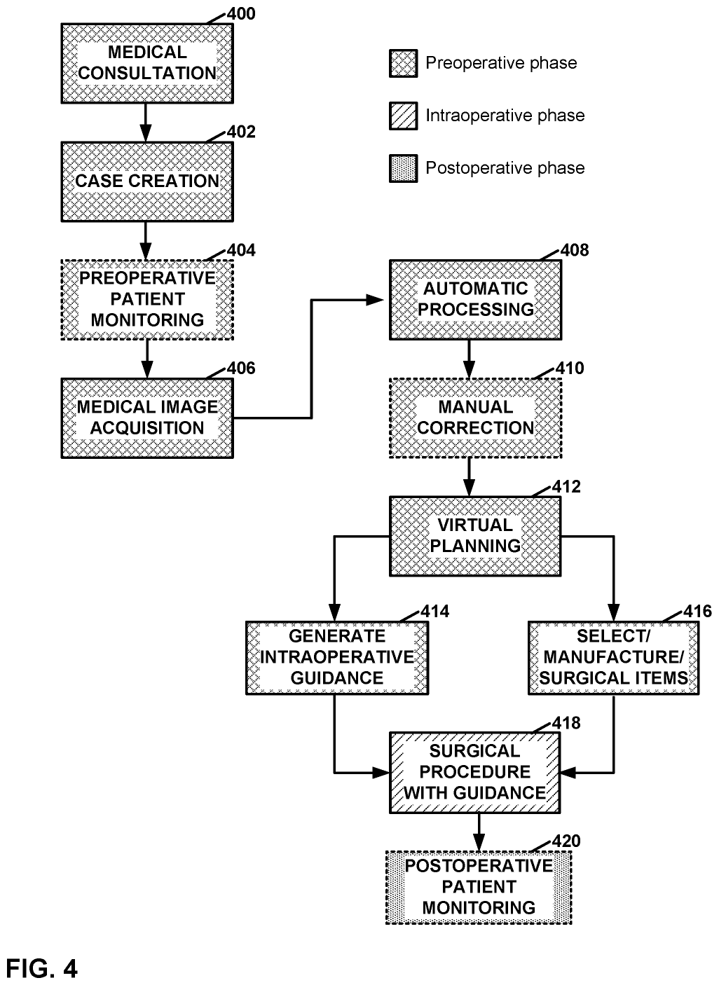

Various workflows may exist within the surgical process of FIG. 3. For example, different workflows within the surgical process of FIG. 3 may be appropriate for different types of surgeries. FIG. 4 is a flowchart illustrating preoperative, intraoperative and postoperative workflows in support of an orthopedic surgical procedure. In the example of FIG. 4, the surgical process begins with a medical consultation (400). During the medical consultation (400), a healthcare professional evaluates a medical condition of a patient. For instance, the healthcare professional may consult the patient with respect to the patient's symptoms. During the medical consultation (400), the healthcare professional may also discuss various treatment options with the patient. For instance, the healthcare professional may describe one or more different surgeries to address the patient's symptoms.

Furthermore, the example of FIG. 4 includes a case creation step (402). In other examples, the case creation step occurs before the medical consultation step. During the case creation step, the medical professional or other user establishes an electronic case file for the patient. The electronic case file for the patient may include information related to the patient, such as data regarding the patient's symptoms, patient range of motion observations, data regarding a surgical plan for the patient, medical images of the patients, notes regarding the patient, billing information regarding the patient, and so on.

The example of FIG. 4 includes a preoperative patient monitoring phase (404). During the preoperative patient monitoring phase, the patient's symptoms may be monitored. For example, the patient may be suffering from pain associated with arthritis in the patient's shoulder. In this example, the patient's symptoms may not yet rise to the level of requiring an arthroplasty to replace the patient's shoulder. However, arthritis typically worsens over time. Accordingly, the patient's symptoms may be monitored to determine whether the time has come to perform a surgery on the patient's shoulder. Observations from the preoperative patient monitoring phase may be stored in the electronic case file for the patient. In some examples, predictive analytics system 114 may be used to predict when the patient may need surgery, to predict a course of treatment to delay or avoid surgery or make other predictions with respect to the patient's health.

Additionally, in the example of FIG. 4, a medical image acquisition step occurs during the preoperative phase (406). During the image acquisition step, medical images of the patient are generated. The medical images may be generated in a variety of ways. For instance, the images may be generated using a Computed Tomography (CT) process, a Magnetic Resonance Imaging (MRI) process, an ultrasound process, or another imaging process. The medical images generated during the image acquisition step include images of an anatomy of interest of the patient. For instance, if the patient's symptoms involve the patient's shoulder, medical images of the patient's shoulder may be generated. The medical images may be added to the patient's electronic case file. Healthcare professionals may be able to use the medical images in one or more of the preoperative, intraoperative, and postoperative phases.

Furthermore, in the example of FIG. 4, an automatic processing step may occur (408). During the automatic processing step, virtual planning system 102 (FIG. 1) may automatically develop a preliminary surgical plan for the patient. In some examples of this disclosure, virtual planning system 102 may use machine learning techniques to develop the preliminary surgical plan based on information in the patient's virtual case file.

The example of FIG. 4 also includes a manual correction step (410). During the manual correction step, one or more human users may check and correct the determinations made during the automatic processing step. In some examples of this disclosure, one or more users may use mixed reality or virtual reality visualization devices during the manual correction step. In some examples, changes made during the manual correction step may be used as training data to refine the machine learning techniques applied by virtual planning system 102 during the automatic processing step.

A virtual planning step (412) may follow the manual correction step in FIG. 4. During the virtual planning step, a healthcare professional may develop a surgical plan for the patient. In some examples of this disclosure, one or more users may use mixed reality or virtual reality visualization devices during development of the surgical plan for the patient.

Furthermore, in the example of FIG. 4, intraoperative guidance may be generated (414). The intraoperative guidance may include guidance to a surgeon on how to execute the surgical plan. In some examples of this disclosure, virtual planning system 102 may generate at least part of the intraoperative guidance. In some examples, the surgeon or other user may contribute to the intraoperative guidance.

Additionally, in the example of FIG. 4, a step of selecting and manufacturing surgical items is performed (416). During the step of selecting and manufacturing surgical items, manufacturing and delivery system 106 (FIG. 1) may manufacture surgical items for use during the surgery described by the surgical plan. For example, the surgical items may include surgical implants, surgical tools, and other items required to perform the surgery described by the surgical plan.

In the example of FIG. 4, a surgical procedure may be performed with guidance from intraoperative system 108 (FIG. 1) (418). For example, a surgeon may perform the surgery while wearing a head-mounted MR visualization device of intraoperative system 108 that presents guidance information to the surgeon. The guidance information may help guide the surgeon through the surgery, providing guidance for various steps in a surgical workflow, including sequence of steps, details of individual steps, and tool or implant selection, implant placement and position, and bone surface preparation for various steps in the surgical procedure workflow.

Postoperative patient monitoring may occur after completion of the surgical procedure (420). During the postoperative patient monitoring step, healthcare outcomes of the patient may be monitored. Healthcare outcomes may include relief from symptoms, ranges of motion, complications, performance of implanted surgical items, and so on. Pre- and postoperative monitoring system 112 (FIG. 1) may assist in the postoperative patient monitoring step.

The medical consultation, case creation, preoperative patient monitoring, image acquisition, automatic processing, manual correction, and virtual planning steps of FIG. 4 are part of preoperative phase 302 of FIG. 3. The surgical procedures with guidance steps of FIG. 4 is part of intraoperative phase 306 of FIG. 3. The postoperative patient monitoring step of FIG. 4 is part of postoperative phase 308 of FIG. 3.

As mentioned above, one or more of the subsystems of orthopedic surgical system 100 may include one or more mixed reality (MR) systems, such as MR system 212 (FIG. 2). Each MR system may include a visualization device. For instance, in the example of FIG. 2, MR system 212 includes visualization device 213. In some examples, in addition to including a visualization device, an MR system may include external computing resources that support the operations of the visualization device. For instance, the visualization device of an MR system may be communicatively coupled to a computing device (e.g., a personal computer, backpack computer, smartphone, etc.) that provides the external computing resources. Alternatively, adequate computing resources may be provided on or within visualization device 213 to perform necessary functions of the visualization device.

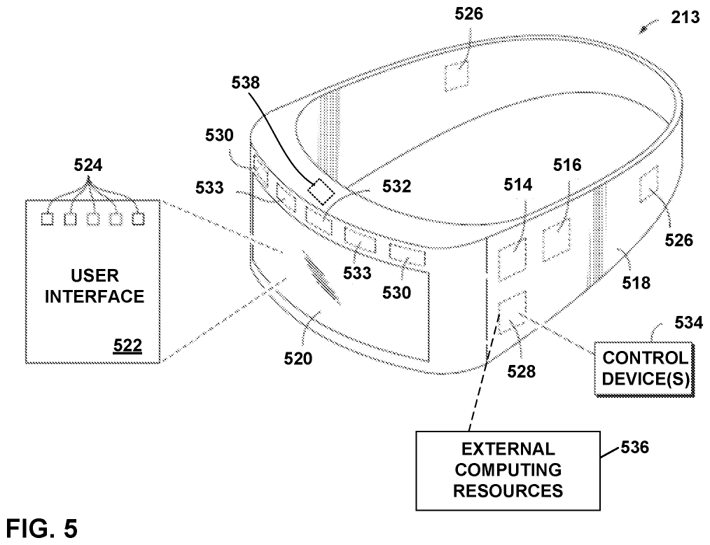

FIG. 5 is a schematic representation of visualization device 213 for use in an MR system, such as MR system 212 of FIG. 2, according to an example of this disclosure. As shown in the example of FIG. 5, visualization device 213 can include a variety of electronic components found in a computing system, including one or more processor(s) 514 (e.g., microprocessors or other types of processing units) and memory 516 that may be mounted on or within a frame 518. Furthermore, in the example of FIG. 5, visualization device 213 may include a transparent screen 520 that is positioned at eye level when visualization device 213 is worn by a user. In some examples, screen 520 can include one or more liquid crystal displays (LCDs) or other types of display screens on which images are perceptible to a surgeon who is wearing or otherwise using visualization device 213 via screen 520. Other display examples include organic light emitting diode (OLED) displays. In some examples, visualization device 213 can operate to project 3D images onto the user's retinas using techniques known in the art.

In some examples, screen 520 may include see-through holographic lenses. sometimes referred to as waveguides, that permit a user to see real-world objects through (e.g., beyond) the lenses and also see holographic imagery projected into the lenses and onto the user's retinas by displays, such as liquid crystal on silicon (LCoS) display devices, which are sometimes referred to as light engines or projectors, operating as an example of a holographic projection system 538 within visualization device 213. In other words, visualization device 213 may include one or more see-through holographic lenses to present virtual images to a user. Hence, in some examples, visualization device 213 can operate to project 3D images onto the user's retinas via screen 520, e.g., formed by holographic lenses. In this manner, visualization device 213 may be configured to present a 3D virtual image to a user within a real-world view observed through screen 520, e.g., such that the virtual image appears to form part of the real-world environment. In some examples, visualization device 213 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses.

Although the example of FIG. 5 illustrates visualization device 213 as a head-wearable device, visualization device 213 may have other forms and form factors. For instance, in some examples, visualization device 213 may be a handheld smartphone or tablet.

Visualization device 213 can also generate a user interface (UI) 522 that is visible to the user, e.g., as holographic imagery projected into see-through holographic lenses as described above. For example, UI 522 can include a variety of selectable widgets 524 that allow the user to interact with a mixed reality (MR) system, such as MR system 212 of FIG. 2. Imagery presented by visualization device 213 may include, for example, one or more 3D virtual objects. Details of an example of UI 522 are described elsewhere in this disclosure. Visualization device 213 also can include a speaker or other sensory devices 526 that may be positioned adjacent the user's ears. Sensory devices 526 can convey audible information or other perceptible information (e.g., vibrations) to assist the user of visualization device 213.

Visualization device 213 can also include a transceiver 528 to connect visualization device 213 to a processing device 510 and/or to network 208 and/or to a computing cloud, such as via a wired communication protocol or a wireless protocol, e.g., Wi-Fi, Bluetooth, etc. Visualization device 213 also includes a variety of sensors to collect sensor data, such as one or more optical camera(s) 530 (or other optical sensors) and one or more depth camera(s) 532 (or other depth sensors), mounted to, on or within frame 518. In some examples, the optical sensor(s) 530 are operable to scan the geometry of the physical environment in which a user of MR system 212 is located (e.g., an operating room) and collect two-dimensional (2D) optical image data (either monochrome or color). Depth sensor(s) 532 are operable to provide 3D image data, such as by employing time of flight, stereo or other known or future-developed techniques for determining depth and thereby generating image data in three dimensions. Other sensors can include motion sensors 533 (e.g., Inertial Mass Unit (IMU) sensors, accelerometers, etc.) to assist with tracking movement.

MR system 212 processes the sensor data so that geometric, environmental, textural, or other types of landmarks (e.g., corners, edges or other lines, walls, floors, objects) in the user's environment or “scene” can be defined and movements within the scene can be detected. As an example, the various types of sensor data can be combined or fused so that the user of visualization device 213 can perceive 3D images that can be positioned, or fixed and/or moved within the scene. When a 3D image is fixed in the scene, the user can walk around the 3D image, view the 3D image from different perspectives, and manipulate the 3D image within the scene using hand gestures, voice commands, gaze line (or direction) and/or other control inputs. As another example, the sensor data can be processed so that the user can position a 3D virtual object (e.g., a bone model) on an observed physical object in the scene (e.g., a surface, the patient's real bone, etc.) and/or orient the 3D virtual object with other virtual images displayed in the scene. In some examples, the sensor data can be processed so that the user can position and fix a virtual representation of the surgical plan (or other widget, image or information) onto a surface, such as a wall of the operating room. Yet further, in some examples, the sensor data can be used to recognize surgical instruments and the position and/or location of those instruments.

Visualization device 213 may include one or more processors 514 and memory 516, e.g., within frame 518 of the visualization device. In some examples, one or more external computing resources 536 process and store information, such as sensor data, instead of or in addition to in-frame processor(s) 514 and memory 516. In this way, data processing and storage may be performed by one or more processors 514 and memory 516 within visualization device 213 and/or some of the processing and storage requirements may be offloaded from visualization device 213. Hence, in some examples, one or more processors that control the operation of visualization device 213 may be within visualization device 213, e.g., as processor(s) 514. Alternatively, in some examples, at least one of the processors that controls the operation of visualization device 213 may be external to visualization device 213, e.g., as processor(s) 210. Likewise, operation of visualization device 213 may, in some examples, be controlled in part by a combination one or more processors 514 within the visualization device and one or more processors 210 external to visualization device 213.

For instance, in some examples, when visualization device 213 is in the context of FIG. 2, processing of the sensor data can be performed by processing device(s) 210 in conjunction with memory or storage device(s) (M) 215. In some examples, processor(s) 514 and memory 516 mounted to frame 518 may provide sufficient computing resources to process the sensor data collected by cameras 530, 532 and motion sensors 533. In some examples, the sensor data can be processed using a Simultaneous Localization and Mapping (SLAM) algorithm, or other known or future-developed algorithms for processing and mapping 2D and 3D image data and tracking the position of visualization device 213 in the 3D scene. In some examples, image tracking may be performed using sensor processing and tracking functionality provided by the Microsoft HOLOLENS™ system, e.g., by one or more sensors and processors 514 within a visualization device 213 substantially conforming to the Microsoft HOLOLENS™ device or a similar mixed reality (MR) visualization device.

In some examples, MR system 212 can also include user-operated control device(s) 534 that allow the user to operate MR system 212, use MR system 212 in spectator mode (either as master or observer), interact with UI 522 and/or otherwise provide commands or requests to processing device(s) 210 or other systems connected to network 208. As examples, control device(s) 534 can include a microphone, a touch pad, a control panel, a motion sensor or other types of control input devices with which the user can interact.

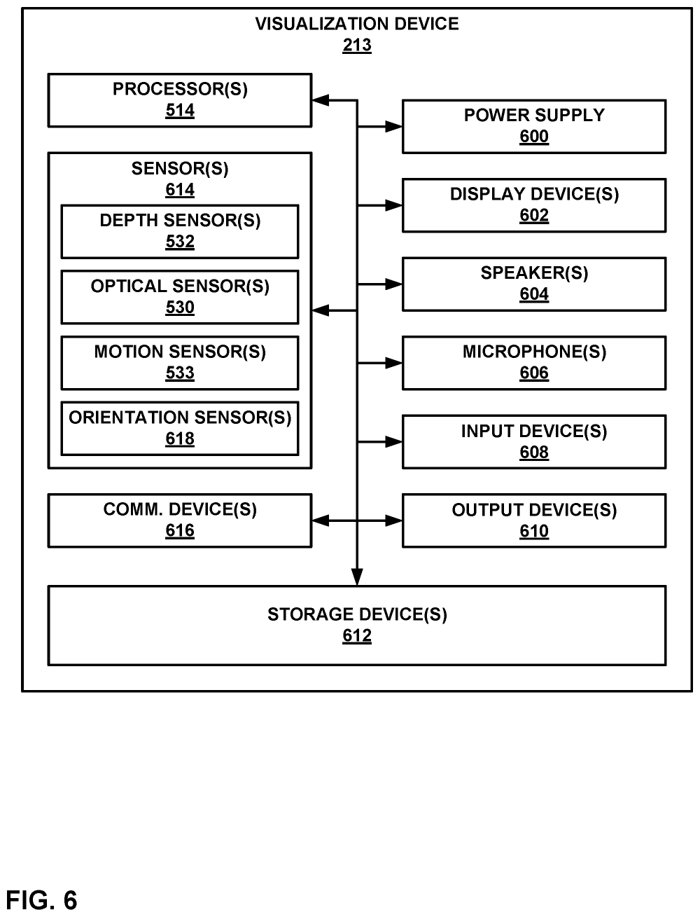

FIG. 6 is a block diagram illustrating example components of visualization device 213 for use in a MR system. In the example of FIG. 6, visualization device 213 includes processors 514, a power supply 600, display device(s) 602, speakers 604, microphone(s) 606, input device(s) 608, output device(s) 610, storage device(s) 612, sensor(s) 614, and communication devices 616. In the example of FIG. 6, sensor(s) 616 may include depth sensor(s) 532, optical sensor(s) 530, motion sensor(s) 533, and orientation sensor(s) 618. Optical sensor(s) 530 may include cameras, such as Red-Green-Blue (RGB) video cameras, infrared cameras, or other types of sensors that form images from light. Display device(s) 602 may display imagery to present a user interface to the user.

Speakers 604, in some examples, may form part of sensory devices 526 shown in FIG. 5. In some examples, display devices 602 may include screen 520 shown in FIG. 5. For example, as discussed with reference to FIG. 5, display device(s) 602 may include see-through holographic lenses, in combination with projectors, that permit a user to see real-world objects, in a real-world environment, through the lenses, and also see virtual 3D holographic imagery projected into the lenses and onto the user's retinas, e.g., by a holographic projection system. In this example, virtual 3D holographic objects may appear to be placed within the real-world environment. In some examples, display devices 602 include one or more display screens, such as LCD display screens, OLED display screens, and so on. The user interface may present virtual images of details of the virtual surgical plan for a particular patient.

In some examples, a user may interact with and control visualization device 213 in a variety of ways. For example, microphones 606, and associated speech recognition processing circuitry or software, may recognize voice commands spoken by the user and, in response, perform any of a variety of operations, such as selection, activation, or deactivation of various functions associated with surgical planning, intra-operative guidance, or the like. As another example, one or more cameras or other optical sensors 530 of sensors 614 may detect and interpret gestures to perform operations as described above. As a further example, sensors 614 may sense gaze direction and perform various operations as described elsewhere in this disclosure. In some examples, input devices 608 may receive manual input from a user, e.g., via a handheld controller including one or more buttons, a keypad, a touchscreen, joystick, trackball, and/or other manual input media, and perform, in response to the manual user input, various operations as described above.

As discussed above, surgical lifecycle 300 may include a preoperative phase 302 (FIG. 3). One or more users may use orthopedic surgical system 100 in preoperative phase 302. For instance, orthopedic surgical system 100 may include virtual planning system 102 to help the one or more users generate a virtual surgical plan that may be customized to an anatomy of interest of a particular patient. As described herein, the virtual surgical plan may include a 3-dimensional virtual model that corresponds to the anatomy of interest of the particular patient and a 3-dimensional model of one or more prosthetic components matched to the particular patient to repair the anatomy of interest or selected to repair the anatomy of interest. The virtual surgical plan also may include a 3-dimensional virtual model of guidance information to guide a surgeon in performing the surgical procedure, e.g., in preparing bone surfaces or tissue and placing implantable prosthetic hardware relative to such bone surfaces or tissue.

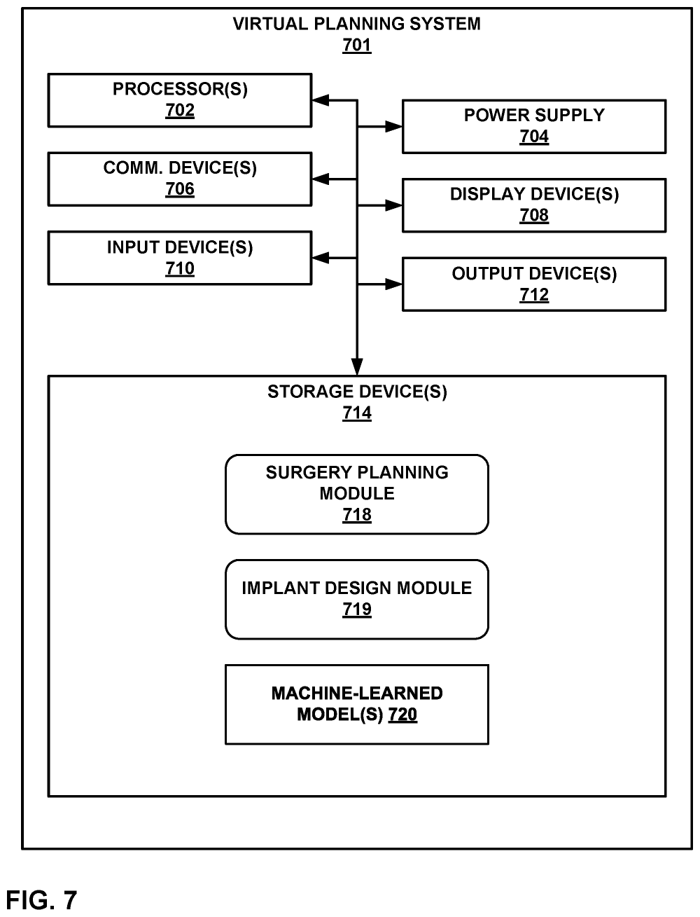

FIG. 7 is a block diagram illustrating example components of virtual planning system 701. Virtual planning system 701 may be considered an example of virtual planning system 102 (FIG. 1 and FIG. 7) or 202 (FIG. 2). Examples of virtual planning system include, but are not limited to, laptops, desktops, server systems, mobile computing devices (e.g., smartphones), wearable computing devices (e.g., head-mounted devices such as visualization device 213 of FIG. 5), or any other computing system or computing component. In the example of FIG. 7, virtual planning system 701 includes processor(s) 702, power supply 704, communication device(s) 706, display device(s) 708, input device(s) 710, output device(s) 712, and storage device(s) 714.

Processor(s) 702 may process information at virtual planning system 701. Processors 702 may be implemented at any variety of circuitry, such as one or more microprocessors, digital signal processors (DSPs), application specific integrated circuits (ASICs), field programmable gate arrays (FPGAs), discrete logic, software, hardware, firmware or any combinations thereof.

Power supply 704 may provide power to one or more components of virtual planning system 701. For example, power supply 704 may provide electrical power to processor(s) 702, communication device(s) 706, display device(s) 708, input device(s) 710, output device(s) 712, and storage device(s) 714.

Communication device(s) 706 may facilitate communication between virtual planning system 701 and various other devices and systems. For instance, communication devices 706 may facilitate communication between virtual planning system 701 and any of planning support system 104, manufacturing and delivery system 106, intraoperative guidance system 108, medical education system 110, monitoring system 112, and predictive analytics system 114 of FIG. 1 (e.g., via network 116 of FIG. 1). Examples of communication devices 706 include, but are not limited to, wired network adaptors (e.g., ethernet adaptors/cards), wireless network adaptors (e.g., Wi-Fi adaptors, cellular network adaptors (e.g., 3G, 4G, LTE, 5G, etc.)), universal serial bus (USB) adaptors, or any other device capable of facilitating inter-device communication.

Display device(s) 708 may be configured to display information to a user of virtual planning system 701. For instance, display devices 708 may display a graphical user interface (GUI) via which virtual planning system 701 may convey information. Examples of display devices 708 include, but are not limited to, liquid crystal displays (LCDs), organic light emitting diode (OLED) displays, plasma displays, projectors, or other types of display screens on which images are perceptible to a user.

Input device(s) 710 may be configured to receive input at virtual planning system 701. Examples of input devices 710 include, but are not limited to, user input devices (e.g., keyboards, mice, microphones, touchscreens, etc.) and sensors (e.g., photosensors, temperature sensors, pressure sensors, etc.).

Output device(s) 712 may be configured to provide output from virtual planning system 701. Examples of output devices 712 include, but are not limited to, speakers, lights, haptic output devices, display devices (e.g., display devices 708 may, in some examples, be considered an output device), communication devices (e.g., communication devices 706 may, in some examples, be considered an output device), or any other device capable of producing a user-perceptible signal.

Storage device(s) 714 may be configured to store information at virtual planning system 102. Examples of storage devices 714 include, but are not limited to, random access memory (RAM), hard drives (e.g., both solid state and not solid state), optical drives, or any other device capable of storing information. In some examples, storage devices 714 may be considered to be non-transitory computer-readable storage media. As shown in FIG. 7, virtual planning system 701 may include surgery planning module 718 and machine-learned model 720.

Surgery planning module 718 may facilitate the planning of surgical procedures. For instance, surgery planning module 718 may facilitate the preoperative creation of a surgical plan. A surgical plan created with surgery planning module 718 may specify one or more of: a surgery type, an implant type, an implant location, and/or any other aspects of a surgical procedure. One example of surgery planning module 718 is the BLUEPRINT™ system.

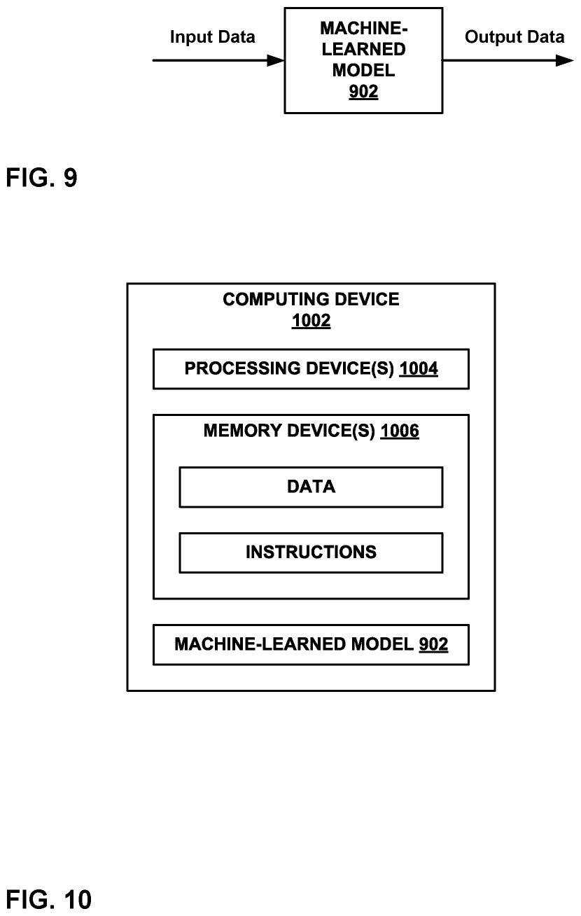

As discussed in further detail below, surgery planning module 718 may invoke/execute or otherwise utilize one or more of machine-learned models 720 to aid in the planning of a surgical procedure. For instance, surgery planning module 718 may invoke a particular machine-learned model of machine-learned models 720 to recommend/predict/estimate a particular aspect of a surgical procedure. As one example, surgery planning module 718 may use one or more of machine-learned models 720 to determine feasibility scores and select one or more implants based on the feasibility scores. As another example, surgery planning module 718 may use one or more of machine-learned models 720 to determine information indicative of dimensions of an implant. As another example, surgery planning module 718 may use one or more of machine-learned models 720 to determine whether a selected surgical option is among a set of recommended surgical options. As another example, surgery planning module 718 may use one or more of machine-learned models 720 to estimate an amount of operating room time for a surgical procedure. Additional details of machine-learned models 720 are discussed below with reference to FIGS. 9-13.

Surgery planning module 718 may perform operations described using software, hardware, firmware, or a mixture of hardware, software, and/or firmware residing in and/or executing at virtual planning system 701. Virtual planning system 701 may execute module 718 and models 720 with one or more of processors 702. Virtual planning system 701 may execute surgery planning module 718 and machine-learned models 720 as a virtual machine executing on underlying hardware. Surgery planning module 718 and machine-learned models 720 may execute as a service or component of an operating system or computing platform. Surgery planning module 718 and machine-learned models 720 may execute as one or more executable programs at an application layer of a computing platform. Surgery planning module 718 and machine-learned models 720 may be otherwise arranged remotely to and remotely accessible to virtual planning system 701, for instance, as one or more network services operating at a network in a network cloud. Although surgery planning module 718 is described as a module, surgery planning module 718 may be implemented using one or more modules or other software architectures.

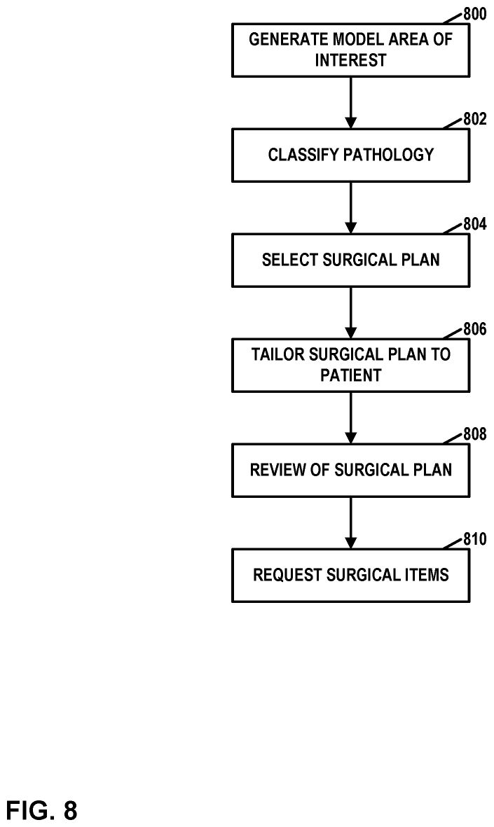

FIG. 8 is a flowchart illustrating example steps in preoperative phase 302 of surgical lifecycle 300. In other examples, preoperative phase 302 may include more, fewer, or different steps. Moreover, in other examples, one or more of the steps of FIG. 8 may be performed in different orders. In some examples, one or more of the steps may be performed automatically within a surgical planning system such as virtual planning system 102 (FIG. 1), 202 (FIG. 2), or 702 (FIG. 7).

In the example of FIG. 8, a model of the area of interest is generated (800). For example, a scan (e.g., a CT scan, MRI scan, or other type of scan) of the area of interest may be performed. For example, if the area of interest is the patient's shoulder, a scan of the patient's shoulder may be performed. Furthermore, a pathology in the area of interest may be classified (802). In some examples, the pathology of the area of interest may be classified based on the scan of the area of interest. For example, if the area of interest is the user's shoulder, a surgeon may determine what is wrong with the patient's shoulder based on the scan of the patient's shoulder and provide a shoulder classification indicating the classification or diagnosis, e.g., such as primary glenoid humeral osteoarthritis (PGHOA), rotator cuff tear arthropathy (RCTA) instability, massive rotator cuff tear (MRCT), rheumatoid arthritis, post-traumatic arthritis, and osteoarthritis.

Additionally, a surgical plan may be selected based on the pathology (804). The surgical plan is a plan to address the pathology. For instance, in the example where the area of interest is the patient's shoulder, the surgical plan may be selected from an anatomical shoulder arthroplasty, a reverse shoulder arthroplasty, a post-trauma shoulder arthroplasty, or a revision to a previous shoulder arthroplasty. The surgical plan may then be tailored to patient (806). For instance, tailoring the surgical plan may involve selecting and/or sizing surgical items needed to perform the selected surgical plan. Additionally, the surgical plan may be tailored to the patient in order to address issues specific to the patient, such as the presence of osteophytes. As described in detail elsewhere in this disclosure, one or more users may use mixed reality systems of orthopedic surgical system 100 to tailor the surgical plan to the patient.

The surgical plan may then be reviewed (808). For instance, a consulting surgeon may review the surgical plan before the surgical plan is executed. As described in detail elsewhere in this disclosure, one or more users may use mixed reality (MR) systems of orthopedic surgical system 100 to review the surgical plan. In some examples, a surgeon may modify the surgical plan using an MR system by interacting with a UI and displayed elements, e.g., to select a different procedure, change the sizing, shape or positioning of implants, or change the angle, depth or amount of cutting or reaming of the bone surface to accommodate an implant.

Additionally, in the example of FIG. 8, surgical items needed to execute the surgical plan may be requested (810). As described in the following sections of this disclosure, orthopedic surgical system 100 may assist various users in performing one or more of the preoperative steps of FIG. 8.

FIGS. 9 through 13 are conceptual diagrams illustrating aspects of an example machine-learned model according to example implementations of the present disclosure. FIGS. 9 through 13 are described below in the context of orthopedic surgical system 100 of FIG. 1. For example, in some instances, machine-learned model 902, as referenced below, may be utilized (e.g., executed, trained, etc.) by any component of orthopedic surgical system 100, such as orthopedic surgical system 100. For instance, machine-learned model 902 may be considered an example of a machine-learned model of machine learned models 720 of FIG. 7

FIG. 9 depicts a conceptual diagram of an example machine-learned model according to example implementations of the present disclosure. As illustrated in FIG. 9, in some implementations, machine-learned model 902 is trained to receive input data of one or more types and, in response, provide output data of one or more types. Thus, FIG. 9 illustrates machine-learned model 902 performing inference.

The input data may include one or more features that are associated with an instance or an example. In some implementations, the one or more features associated with the instance or example can be organized into a feature vector. In some implementations, the output data can include one or more predictions. Predictions can also be referred to as inferences. Thus, given features associated with a particular instance, machine-learned model 902 can output a prediction for such instance based on the features.

Machine-learned model 902 can be or include one or more of various different types of machine-learned models. In particular, in some implementations, machine-learned model 902 can perform classification, regression, clustering, anomaly detection, recommendation generation, and/or other tasks.

In some implementations, machine-learned model 902 can perform various types of classification based on the input data. For example, machine-learned model 902 can perform binary classification or multiclass classification. In binary classification, the output data can include a classification of the input data into one of two different classes. In multiclass classification, the output data can include a classification of the input data into one (or more) of more than two classes. The classifications can be single label or multi-label. Machine-learned model 902 may perform discrete categorical classification in which the input data is simply classified into one or more classes or categories.

In some implementations, machine-learned model 902 can perform classification in which machine-learned model 902 provides, for each of one or more classes, a numerical value descriptive of a degree to which it is believed that the input data should be classified into the corresponding class. In some instances, the numerical values provided by machine-learned model 902 can be referred to as “confidence scores” that are indicative of a respective confidence associated with classification of the input into the respective class. In some implementations, the confidence scores can be compared to one or more thresholds to render a discrete categorical prediction. In some implementations, only a certain number of classes (e.g., one) with the relatively largest confidence scores can be selected to render a discrete categorical prediction.

Machine-learned model 902 may output a probabilistic classification. For example, machine-learned model 902 may predict, given a sample input, a probability distribution over a set of classes. Thus, rather than outputting only the most likely class to which the sample input should belong, machine-learned model 902 can output, for each class, a probability that the sample input belongs to such class. In some implementations, the probability distribution over all possible classes can sum to one. In some implementations, a Softmax function, or other type of function or layer can be used to squash a set of real values respectively associated with the possible classes to a set of real values in the range (0, 1) that sum to one.

In some examples, the probabilities provided by the probability distribution can be compared to one or more thresholds to render a discrete categorical prediction. In some implementations, only a certain number of classes (e.g., one) with the relatively largest predicted probability can be selected to render a discrete categorical prediction.

In cases in which machine-learned model 902 performs classification, machine-learned model 902 may be trained using supervised learning techniques. For example, machine-learned model 902 may be trained on a training dataset that includes training examples labeled as belonging (or not belonging) to one or more classes. Further details regarding supervised training techniques are provided below in the descriptions of FIGS. 10-13.

In some implementations, machine-learned model 902 can perform regression to provide output data in the form of a continuous numeric value. The continuous numeric value can correspond to any number of different metrics or numeric representations, including, for example, currency values, scores, or other numeric representations. As examples, machine-learned model 902 can perform linear regression, polynomial regression, or nonlinear regression. As examples, machine-learned model 902 can perform simple regression or multiple regression. As described above, in some implementations, a Softmax function or other function or layer can be used to squash a set of real values respectively associated with a two or more possible classes to a set of real values in the range (0, 1) that sum to one.

Machine-learned model 902 may perform various types of clustering. For example, machine-learned model 902 can identify one or more previously-defined clusters to which the input data most likely corresponds. Machine-learned model 902 may identify one or more clusters within the input data. That is, in instances in which the input data includes multiple objects, documents, or other entities, machine-learned model 902 can sort the multiple entities included in the input data into a number of clusters. In some implementations in which machine-learned model 902 performs clustering, machine-learned model 902 can be trained using unsupervised learning techniques.

Machine-learned model 902 may perform anomaly detection or outlier detection. For example, machine-learned model 902 can identify input data that does not conform to an expected pattern or other characteristic (e.g., as previously observed from previous input data). As examples, the anomaly detection can be used for fraud detection or system failure detection.

In some implementations, machine-learned model 902 can provide output data in the form of one or more recommendations. For example, machine-learned model 902 can be included in a recommendation system or engine. As an example, given input data that describes previous outcomes for certain entities (e.g., a score, ranking, or rating indicative of an amount of success or enjoyment), machine-learned model 902 can output a suggestion or recommendation of one or more additional entities that, based on the previous outcomes, are expected to have a desired outcome (e.g., elicit a score, ranking, or rating indicative of success or enjoyment). As one example, given input data descriptive of a patient, a recommendation system, such as orthopedic surgical system 100 of FIG. 1, can output a suggestion or recommendation of a surgical procedure or one or more aspects of a surgical procedure to be performed on the patient.

Machine-learned model 902 may, in some cases, act as an agent within an environment. For example, machine-learned model 902 can be trained using reinforcement learning, which will be discussed in further detail below.

In some implementations, machine-learned model 902 can be a parametric model while, in other implementations, machine-learned model 902 can be a non-parametric model. In some implementations, machine-learned model 902 can be a linear model while, in other implementations, machine-learned model 902 can be a non-linear model.

As described above, machine-learned model 902 can be or include one or more of various different types of machine-learned models. Examples of such different types of machine-learned models are provided below for illustration. One or more of the example models described below can be used (e.g., combined) to provide the output data in response to the input data. Additional models beyond the example models provided below can be used as well.

In some implementations, machine-learned model 902 can be or include one or more classifier models such as, for example, linear classification models; quadratic classification models; etc. Machine-learned model 902 may be or include one or more regression models such as, for example, simple linear regression models; multiple linear regression models; logistic regression models; stepwise regression models; multivariate adaptive regression splines; locally estimated scatterplot smoothing models; etc.

In some examples, machine-learned model 902 can be or include one or more decision tree-based models such as, for example, classification and/or regression trees; iterative dichotomiser 3 decision trees; C4.5 decision trees; chi-squared automatic interaction detection decision trees; decision stumps; conditional decision trees; etc.

Machine-learned model 902 may be or include one or more kernel machines. In some implementations, machine-learned model 902 can be or include one or more support vector machines. Machine-learned model 902 may be or include one or more instance-based learning models such as, for example, learning vector quantization models; self-organizing map models; locally weighted learning models; etc. In some implementations, machine-learned model 902 can be or include one or more nearest neighbor models such as, for example, k-nearest neighbor classifications models; k-nearest neighbors regression models; etc. Machine-learned model 902 can be or include one or more Bayesian models such as, for example, naive Bayes models; Gaussian naive Bayes models; multinomial naive Bayes models; averaged one-dependence estimators; Bayesian networks; Bayesian belief networks; hidden Markov models; etc.

In some implementations, machine-learned model 902 can be or include one or more artificial neural networks (also referred to simply as neural networks). A neural network can include a group of connected nodes, which also can be referred to as neurons or perceptrons. A neural network can be organized into one or more layers. Neural networks that include multiple layers can be referred to as “deep” networks. A deep network can include an input layer, an output layer, and one or more hidden layers positioned between the input layer and the output layer. The nodes of the neural network can be connected or non-fully connected.

Machine-learned model 902 can be or include one or more feed forward neural networks. In feed forward networks, the connections between nodes do not form a cycle. For example, each connection can connect a node from an earlier layer to a node from a later layer.

In some instances, machine-learned model 902 can be or include one or more recurrent neural networks. In some instances, at least some of the nodes of a recurrent neural network can form a cycle. Recurrent neural networks can be especially useful for processing input data that is sequential in nature. In particular, in some instances, a recurrent neural network can pass or retain information from a previous portion of the input data sequence to a subsequent portion of the input data sequence through the use of recurrent or directed cyclical node connections.

In some examples, sequential input data can include time-series data (e.g., sensor data versus time or imagery captured at different times). For example, a recurrent neural network can analyze sensor data versus time to detect or predict a swipe direction, to perform handwriting recognition, etc. Sequential input data may include words in a sentence (e.g., for natural language processing, speech detection or processing, etc.); notes in a musical composition; sequential actions taken by a user (e.g., to detect or predict sequential application usage); sequential object states; etc.

Example recurrent neural networks include long short-term (LSTM) recurrent neural networks; gated recurrent units; bi-direction recurrent neural networks; continuous time recurrent neural networks; neural history compressors; echo state networks; Elman networks; Jordan networks; recursive neural networks; Hopfield networks; fully recurrent networks; sequence-to-sequence configurations; etc.

In some implementations, machine-learned model 902 can be or include one or more convolutional neural networks. In some instances, a convolutional neural network can include one or more convolutional layers that perform convolutions over input data using learned filters.

Filters can also be referred to as kernels. Convolutional neural networks can be especially useful for vision problems such as when the input data includes imagery such as still images or video. However, convolutional neural networks can also be applied for natural language processing.

In some examples, machine-learned model 902 can be or include one or more generative networks such as, for example, generative adversarial networks. Generative networks can be used to generate new data such as new images or other content.

Machine-learned model 902 may be or include an autoencoder. In some instances, the aim of an autoencoder is to learn a representation (e.g., a lower-dimensional encoding) for a set of data, typically for the purpose of dimensionality reduction. For example, in some instances, an autoencoder can seek to encode the input data and provide output data that reconstructs the input data from the encoding. An autoencoder may be used for learning generative models of data. In some instances, the autoencoder can include additional losses beyond reconstructing the input data.

Machine-learned model 902 may be or include one or more other forms of artificial neural networks such as, for example, deep Boltzmann machines; deep belief networks; stacked autoencoders; etc. Any of the neural networks described herein can be combined (e.g., stacked) to form more complex networks.

One or more neural networks can be used to provide an embedding based on the input data. For example, the embedding can be a representation of knowledge abstracted from the input data into one or more learned dimensions. In some instances, embeddings can be a useful source for identifying related entities. In some instances, embeddings can be extracted from the output of the network, while in other instances embeddings can be extracted from any hidden node or layer of the network (e.g., a close to final but not final layer of the network). Embeddings can be useful for performing auto suggest next video, product suggestion, entity or object recognition, etc. In some instances, embeddings be useful inputs for downstream models. For example, embeddings can be useful to generalize input data (e.g., search queries) for a downstream model or processing system.

Machine-learned model 902 may include one or more clustering models such as, for example, k-means clustering models; k-medians clustering models; expectation maximization models; hierarchical clustering models; etc.

In some implementations, machine-learned model 902 can perform one or more dimensionality reduction techniques such as, for example, principal component analysis; kernel principal component analysis; graph-based kernel principal component analysis; principal component regression; partial least squares regression; Sammon mapping; multidimensional scaling; projection pursuit; linear discriminant analysis; mixture discriminant analysis; quadratic discriminant analysis; generalized discriminant analysis; flexible discriminant analysis; autoencoding; etc.

In some implementations, machine-learned model 902 can perform or be subjected to one or more reinforcement learning techniques such as Markov decision processes; dynamic programming; Q functions or Q-learning; value function approaches; deep Q-networks; differentiable neural computers; asynchronous advantage actor-critics; deterministic policy gradient; etc.

In some implementations, machine-learned model 902 can be an autoregressive model. In some instances, an autoregressive model can specify that the output data depends linearly on its own previous values and on a stochastic term. In some instances, an autoregressive model can take the form of a stochastic difference equation. One example autoregressive model is WaveNet, which is a generative model for raw audio.

In some implementations, machine-learned model 902 can include or form part of a multiple model ensemble. As one example, bootstrap aggregating can be performed, which can also be referred to as “bagging.” In bootstrap aggregating, a training dataset is split into a number of subsets (e.g., through random sampling with replacement) and a plurality of models are respectively trained on the number of subsets. At inference time, respective outputs of the plurality of models can be combined (e.g., through averaging, voting, or other techniques) and used as the output of the ensemble.

One example ensemble is a random forest, which can also be referred to as a random decision forest. Random forests are an ensemble learning method for classification, regression, and other tasks. Random forests are generated by producing a plurality of decision trees at training time. In some instances, at inference time, the class that is the mode of the classes (classification) or the mean prediction (regression) of the individual trees can be used as the output of the forest. Random decision forests can correct for decision trees' tendency to overfit their training set.

Another example ensemble technique is stacking, which can, in some instances, be referred to as stacked generalization. Stacking includes training a combiner model to blend or otherwise combine the predictions of several other machine-learned models. Thus, a plurality of machine-learned models (e.g., of same or different type) can be trained based on training data. In addition, a combiner model can be trained to take the predictions from the other machine-learned models as inputs and, in response, produce a final inference or prediction. In some instances, a single-layer logistic regression model can be used as the combiner model.

Another example ensemble technique is boosting. Boosting can include incrementally building an ensemble by iteratively training weak models and then adding to a final strong model. For example, in some instances, each new model can be trained to emphasize the training examples that previous models misinterpreted (e.g., misclassified). For example, a weight associated with each of such misinterpreted examples can be increased. One common implementation of boosting is AdaBoost, which can also be referred to as Adaptive Boosting. Other example boosting techniques include LPBoost; TotalBoost; BrownBoost; xgboost; MadaBoost, LogitBoost, gradient boosting; etc. Furthermore, any of the models described above (e.g., regression models and artificial neural networks) can be combined to form an ensemble. As an example, an ensemble can include a top level machine-learned model or a heuristic function to combine and/or weight the outputs of the models that form the ensemble.

In some implementations, multiple machine-learned models (e.g., that form an ensemble can be linked and trained jointly (e.g., through backpropagation of errors sequentially through the model ensemble). However, in some implementations, only a subset (e.g., one) of the jointly trained models is used for inference.

In some implementations, machine-learned model 902 can be used to preprocess the input data for subsequent input into another model. For example, machine-learned model 902 can perform dimensionality reduction techniques and embeddings (e.g., matrix factorization, principal components analysis, singular value decomposition, word2vec/GLOVE, and/or related approaches); clustering; and even classification and regression for downstream consumption. Many of these techniques have been discussed above and will be further discussed below.

As discussed above, machine-learned model 902 can be trained or otherwise configured to receive the input data and, in response, provide the output data. The input data can include different types, forms, or variations of input data. As examples, in various implementations, the input data can include features that describe the content (or portion of content) initially selected by the user, e.g., content of user-selected document or image, links pointing to the user selection, links within the user selection relating to other files available on device or cloud, metadata of user selection, etc. Additionally, with user permission, the input data includes the context of user usage, either obtained from app itself or from other sources. Examples of usage context include breadth of share (sharing publicly, or with a large group, or privately, or a specific person), context of share, etc. When permitted by the user, additional input data can include the state of the device, e.g., the location of the device, the apps running on the device, etc.

One example way in which to receive the input data is through an application programming interface (API). As an example, the input data may be stored in a cloud for one or more hospitals. An endpoint for the cloud may retrieve data stored in the cloud in response to a request formatted in accordance with the API for the cloud. Processor(s) 702 may generate the request for specific data stored in the cloud in accordance with the API for the cloud, and communication device(s) 706 may transmit the request to the endpoint for the cloud. In return, communication device(s) 706 may receive the requested data that processor(s) 702 stores as the input data for training machine-learned model 902.

Utilization of the API for accessing the input data may be beneficial for various reasons, such as protecting patient privacy. The API may not allow for a query to access private information that can identify a patient, such as name, address, etc. Hence, the endpoint may not access the private information from the cloud. Accordingly, when training machine-learned model 902, the input data may be limited to protect patient privacy.

In some implementations, machine-learned model 902 can receive and use the input data in its raw form. In some implementations, the raw input data can be preprocessed. Thus, in addition or alternatively to the raw input data, machine-learned model 902 can receive and use the preprocessed input data.

In some implementations, preprocessing the input data can include extracting one or more additional features from the raw input data. For example, feature extraction techniques can be applied to the input data to generate one or more new, additional features. Example feature extraction techniques include edge detection; corner detection; blob detection; ridge detection; scale-invariant feature transform; motion detection; optical flow; Hough transform; etc.

In some implementations, the extracted features can include or be derived from transformations of the input data into other domains and/or dimensions. As an example, the extracted features can include or be derived from transformations of the input data into the frequency domain. For example, wavelet transformations and/or fast Fourier transforms can be performed on the input data to generate additional features.

In some implementations, the extracted features can include statistics calculated from the input data or certain portions or dimensions of the input data. Example statistics include the mode, mean, maximum, minimum, or other metrics of the input data or portions thereof

In some implementations, as described above, the input data can be sequential in nature. In some instances, the sequential input data can be generated by sampling or otherwise segmenting a stream of input data. As one example, frames can be extracted from a video. In some implementations, sequential data can be made non-sequential through summarization.

As another example preprocessing technique, portions of the input data can be imputed. For example, additional synthetic input data can be generated through interpolation and/or extrapolation.

As another example preprocessing technique, some or all of the input data can be scaled, standardized, normalized, generalized, and/or regularized. Example regularization techniques include ridge regression; least absolute shrinkage and selection operator (LASSO); elastic net; least-angle regression; cross-validation; L1 regularization; L2 regularization; etc. As one example, some or all of the input data can be normalized by subtracting the mean across a given dimension's feature values from each individual feature value and then dividing by the standard deviation or other metric.

As another example preprocessing technique, some or all or the input data can be quantized or discretized. In some cases, qualitative features or variables included in the input data can be converted to quantitative features or variables. For example, one hot encoding can be performed.

In some examples, dimensionality reduction techniques can be applied to the input data prior to input into machine-learned model 902. Several examples of dimensionality reduction techniques are provided above, including, for example, principal component analysis; kernel principal component analysis; graph-based kernel principal component analysis; principal component regression; partial least squares regression; Sammon mapping; multidimensional scaling; projection pursuit; linear discriminant analysis; mixture discriminant analysis; quadratic discriminant analysis; generalized discriminant analysis; flexible discriminant analysis; autoencoding; etc.

In some implementations, during training, the input data can be intentionally deformed in any number of ways to increase model robustness, generalization, or other qualities. Example techniques to deform the input data include adding noise; changing color, shade, or hue; magnification; segmentation; amplification; etc.

In response to receipt of the input data, machine-learned model 902 can provide the output data. The output data can include different types, forms, or variations of output data. As examples, in various implementations, the output data can include content, either stored locally on the user device or in the cloud, that is relevantly shareable along with the initial content selection.

As discussed above, in some implementations, the output data can include various types of classification data (e.g., binary classification, multiclass classification, single label, multi-label, discrete classification, regressive classification, probabilistic classification, etc.) or can include various types of regressive data (e.g., linear regression, polynomial regression, nonlinear regression, simple regression, multiple regression, etc.). In other instances, the output data can include clustering data, anomaly detection data, recommendation data, or any of the other forms of output data discussed above.

In some implementations, the output data can influence downstream processes or decision making. As one example, in some implementations, the output data can be interpreted and/or acted upon by a rules-based regulator.

The present disclosure provides systems and methods that include or otherwise leverage one or more machine-learned models to suggest content, either stored locally on the uses device or in the cloud, that is relevantly shareable along with the initial content selection based on features of the initial content selection. Any of the different types or forms of input data described above can be combined with any of the different types or forms of machine-learned models described above to provide any of the different types or forms of output data described above.

The systems and methods of the present disclosure can be implemented by or otherwise executed on one or more computing devices. Example computing devices include user computing devices (e.g., laptops, desktops, and mobile computing devices such as tablets, smartphones, wearable computing devices, etc.); embedded computing devices (e.g., devices embedded within a vehicle, camera, medical scanner, image sensor, industrial machine, satellite, gaming console or controller, or home appliance such as a refrigerator, thermostat, energy meter, home energy manager, smart home assistant, etc.); server computing devices (e.g., database servers, parameter servers, file servers, mail servers, print servers, web servers, game servers, application servers, etc.); dedicated, specialized model processing or training devices; virtual computing devices; other computing devices or computing infrastructure; or combinations thereof.

FIG. 10 illustrates a conceptual diagram of computing device 1002, which is an example of virtual planning system 701 of FIG. 2. Computing device 1002 includes processing component 302, memory component 304 and machine-learned model 902. Computing device 1002 may store and implement machine-learned model 902 locally (i.e., on-device). Thus, in some implementations, machine-learned model 902 can be stored at and/or implemented locally by an embedded device or a user computing device such as a mobile device. Output data obtained through local implementation of machine-learned model 902 at the embedded device or the user computing device can be used to improve performance of the embedded device or the user computing device (e.g., an application implemented by the embedded device or the user computing device).

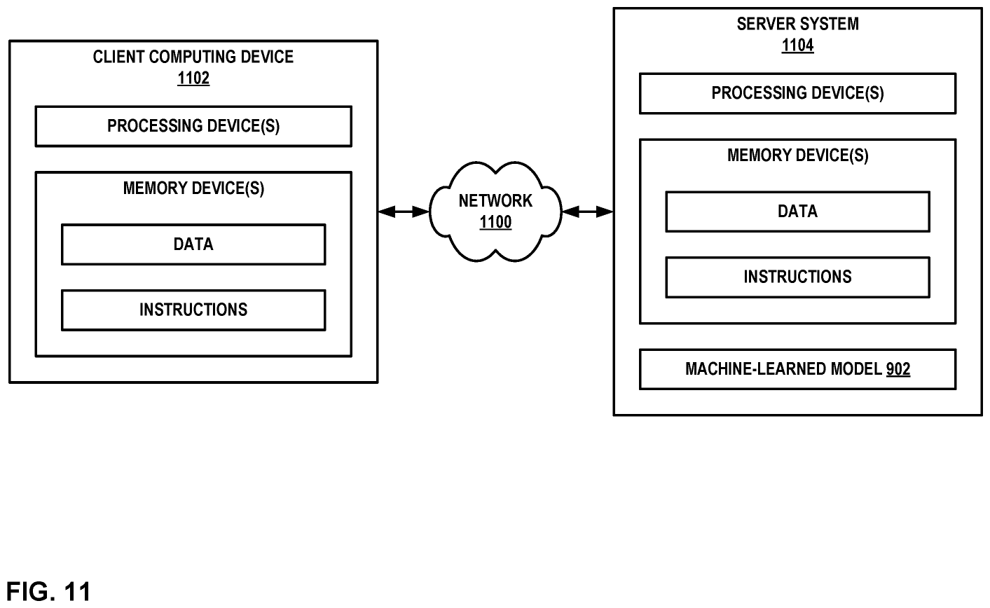

FIG. 11 illustrates a conceptual diagram of an example client computing device that can communicate over a network with an example server computing system that includes a machine-learned model. FIG. 11 includes client computing device 1102 communicating with server system 1104 over network 1100. Client computing device 1102 is an example of virtual planning system 701 of FIG. 2, server system 1104 is an example of any component of orthopedic surgical system 100, and network 1100 is an example of network 116 of FIG. 1. Server system 1104 stores and implements machine-learned model 902. In some instances, output data obtained through machine-learned model 902 at server system 1104 can be used to improve other server tasks or can be used by other non-user devices to improve services performed by or for such other non-user devices. For example, the output data can improve other downstream processes performed by server system 1104 for a computing device of a user or embedded computing device. In other instances, output data obtained through implementation of machine-learned model 902 at server system 1104 can be sent to and used by a user computing device, an embedded computing device, or some other client device, such as client computing device 1102. For example, server system 1104 can be said to perform machine learning as a service.

In yet other implementations, different respective portions of machine-learned model 902 can be stored at and/or implemented by some combination of a user computing device; an embedded computing device; a server computing device; etc. In other words, portions of machine-learned model 902 may be distributed in whole or in part amongst client device 1102 and server system 1104.

Devices 1102 and 1104 may perform graph processing techniques or other machine learning techniques using one or more machine learning platforms, frameworks, and/or libraries, such as, for example, TensorFlow, Caffe/Caffe2, Theano, Torch/PyTorch, MXnet, CNTK, etc. Devices 1102 and 1104 may be distributed at different physical locations and connected via one or more networks, including network 1100. If configured as distributed computing devices, Devices 1102 and 1104 may operate according to sequential computing architectures, parallel computing architectures, or combinations thereof. In one example, distributed computing devices can be controlled or guided through use of a parameter server.

In some implementations, multiple instances of machine-learned model 902 can be parallelized to provide increased processing throughput. For example, the multiple instances of machine-learned model 902 can be parallelized on a single processing device or computing device or parallelized across multiple processing devices or computing devices.

Each computing device that implements machine-learned model 902 or other aspects of the present disclosure can include a number of hardware components that enable performance of the techniques described herein. For example, each computing device can include one or more memory devices that store some or all of machine-learned model 902. For example, machine-learned model 902 can be a structured numerical representation that is stored in memory. The one or more memory devices can also include instructions for implementing machine-learned model 902 or performing other operations. Example memory devices include RAM, ROM, EEPROM, EPROM, flash memory devices, magnetic disks, etc., and combinations thereof.

Each computing device can also include one or more processing devices that implement some or all of machine-learned model 902 and/or perform other related operations. Example processing devices include one or more of: a central processing unit (CPU); a visual processing unit (VPU); a graphics processing unit (GPU); a tensor processing unit (TPU); a neural processing unit (NPU); a neural processing engine; a core of a CPU, VPU, GPU, TPU, NPU or other processing device; an application specific integrated circuit (ASIC); a field programmable gate array (FPGA); a co-processor; a controller; or combinations of the processing devices described above. Processing devices can be embedded within other hardware components such as, for example, an image sensor, accelerometer, etc.

Hardware components (e.g., memory devices and/or processing devices) can be spread across multiple physically distributed computing devices and/or virtually distributed computing systems.

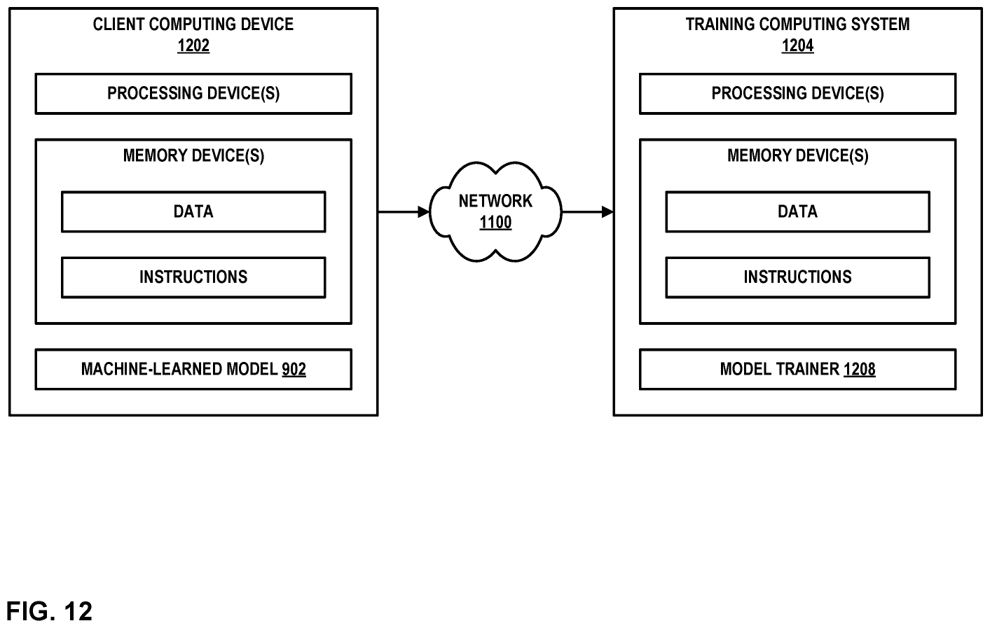

FIG. 12 illustrates a conceptual diagram of an example computing device in communication with an example training computing system that includes a model trainer. FIG. 12 includes client computing device 1202 communicating with training computing system 1204 370 over network 1100. Client computing device 1202 is an example of virtual planning system 701 of FIG. 7 and network 1100 is an example of network 116 of FIG. 1. Machine-learned model 902 described herein can be trained at a training computing system, such as training computing system 1204, and then provided for storage and/or implementation at one or more computing devices, such as client computing device 1202. For example, model trainer 1208 executes locally at training computing system 1204. However, in some examples, training computing system 1204, including model trainer 1208, can be included in or separate from client computing device 1202 or any other computing device that implement machine-learned model 902.

In some implementations, machine-learned model 902 may be trained in an offline fashion or an online fashion. In offline training (also known as batch learning), machine-learned model 902 is trained on the entirety of a static set of training data. In online learning, machine-learned model 902 is continuously trained (or re-trained) as new training data becomes available (e.g., while the model is used to perform inference).

Model trainer 1208 may perform centralized training of machine-learned model 902 (e.g., based on a centrally stored dataset). In other implementations, decentralized training techniques such as distributed training, federated learning, or the like can be used to train, update, or personalize machine-learned model 902.

Machine-learned model 902 described herein can be trained according to one or more of various different training types or techniques. For example, in some implementations, machine-learned model 902 can be trained by model trainer 1208 using supervised learning, in which machine-learned model 902 is trained on a training dataset that includes instances or examples that have labels. The labels can be manually applied by experts, generated through crowdsourcing, or provided by other techniques (e.g., by physics-based or complex mathematical models). In some implementations, if the user has provided consent, the training examples can be provided by the user computing device. In some implementations, this process can be referred to as personalizing the model.

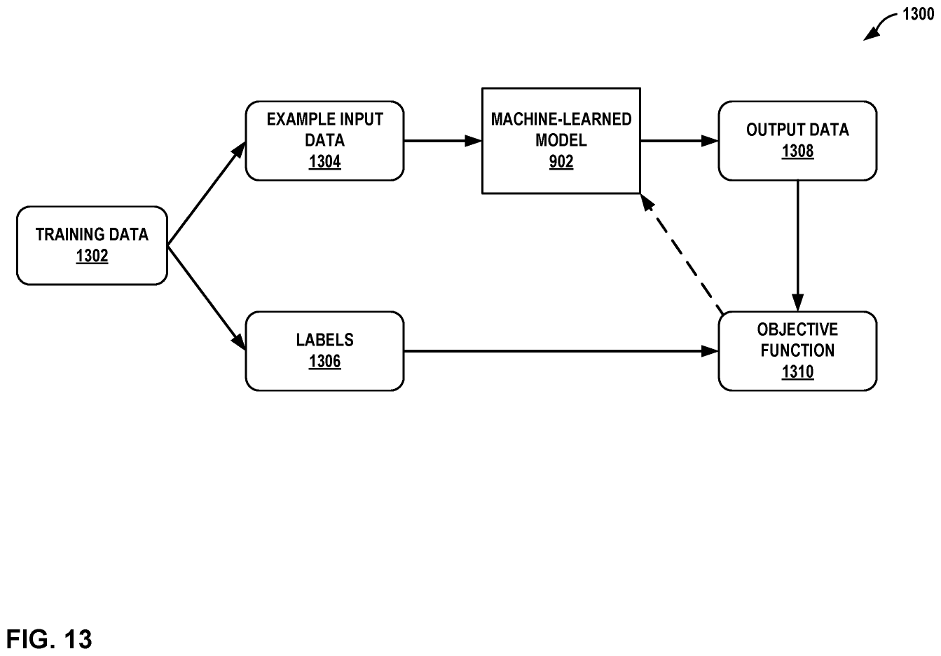

FIG. 13 illustrates a conceptual diagram of training process 1300 which is an example training process in which machine-learned model 902 is trained on training data 1302 that includes example input data 1304 that has labels 1306. Training processes 1300 is one example training process; other training processes may be used as well.

Training data 1302 used by training process 1300 can include, upon user permission for use of such data for training, anonymized usage logs of sharing flows, e.g., content items that were shared together, bundled content pieces already identified as belonging together, e.g., from entities in a knowledge graph, etc. In some implementations, training data 1302 can include examples of input data 1304 that have been assigned labels 1306 that correspond to output data 1308.

In some implementations, machine-learned model 902 can be trained by optimizing an objective function, such as objective function 1310. For example, in some implementations, objective function 1310 may be or include a loss function that compares (e.g., determines a difference between) output data generated by the model from the training data and labels (e.g., ground-truth labels) associated with the training data. For example, the loss function can evaluate a sum or mean of squared differences between the output data and the labels. In some examples, objective function 1310 may be or include a cost function that describes a cost of a certain outcome or output data. Other examples of objective function 1310 can include margin-based techniques such as, for example, triplet loss or maximum-margin training.

One or more of various optimization techniques can be performed to optimize objective function 1310. For example, the optimization technique(s) can minimize or maximize objective function 1310. Example optimization techniques include Hessian-based techniques and gradient-based techniques, such as, for example, coordinate descent; gradient descent (e.g., stochastic gradient descent); subgradient methods; etc. Other optimization techniques include black box optimization techniques and heuristics.

In some implementations, backward propagation of errors can be used in conjunction with an optimization technique (e.g., gradient based techniques) to train machine-learned model 902 (e.g., when machine-learned model is a multi-layer model such as an artificial neural network). For example, an iterative cycle of propagation and model parameter (e.g., weights) update can be performed to train machine-learned model 902. Example backpropagation techniques include truncated backpropagation through time, Levenberg-Marquardt backpropagation, etc.

In some implementations, machine-learned model 902 described herein can be trained using unsupervised learning techniques. Unsupervised learning can include inferring a function to describe hidden structure from unlabeled data. For example, a classification or categorization may not be included in the data. Unsupervised learning techniques can be used to produce machine-learned models capable of performing clustering, anomaly detection, learning latent variable models, or other tasks.

Machine-learned model 902 can be trained using semi-supervised techniques which combine aspects of supervised learning and unsupervised learning. Machine-learned model 902 can be trained or otherwise generated through evolutionary techniques or genetic algorithms. In some implementations, machine-learned model 902 described herein can be trained using reinforcement learning. In reinforcement learning, an agent (e.g., model) can take actions in an environment and learn to maximize rewards and/or minimize penalties that result from such actions. Reinforcement learning can differ from the supervised learning problem in that correct input/output pairs are not presented, nor sub-optimal actions explicitly corrected.

In some implementations, one or more generalization techniques can be performed during training to improve the generalization of machine-learned model 902. Generalization techniques can help reduce overfitting of machine-learned model 902 to the training data. Example generalization techniques include dropout techniques; weight decay techniques; batch normalization; early stopping; subset selection; stepwise selection; etc.

In some implementations, machine-learned model 902 described herein can include or otherwise be impacted by a number of hyperparameters, such as, for example, learning rate, number of layers, number of nodes in each layer, number of leaves in a tree, number of clusters; etc. Hyperparameters can affect model performance. Hyperparameters can be hand selected or can be automatically selected through application of techniques such as, for example, grid search; black box optimization techniques (e.g., Bayesian optimization, random search, etc.); gradient-based optimization; etc. Example techniques and/or tools for performing automatic hyperparameter optimization include Hyperopt; Auto-WEKA; Spearmint; Metric Optimization Engine (MOE); etc.

In some implementations, various techniques can be used to optimize and/or adapt the learning rate when the model is trained. Example techniques and/or tools for performing learning rate optimization or adaptation include Adagrad; Adaptive Moment Estimation (ADAM); Adadelta; RMSprop; etc.

In some implementations, transfer learning techniques can be used to provide an initial model from which to begin training of machine-learned model 902 described herein.

In some implementations, machine-learned model 902 described herein can be included in different portions of computer-readable code on a computing device. In one example, machine-learned model 902 can be included in a particular application or program and used (e.g., exclusively) by such particular application or program. Thus, in one example, a computing device can include a number of applications and one or more of such applications can contain its own respective machine learning library and machine-learned model(s).

In another example, machine-learned model 902 described herein can be included in an operating system of a computing device (e.g., in a central intelligence layer of an operating system) and can be called or otherwise used by one or more applications that interact with the operating system. In some implementations, each application can communicate with the central intelligence layer (and model(s) stored therein) using an application programming interface (API) (e.g., a common, public API across all applications).

In some implementations, the central intelligence layer can communicate with a central device data layer. The central device data layer can be a centralized repository of data for the computing device. The central device data layer can communicate with a number of other components of the computing device, such as, for example, one or more sensors, a context manager, a device state component, and/or additional components. In some implementations, the central device data layer can communicate with each device component using an API (e.g., a private API).

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination.

Databases and applications can be implemented on a single system or distributed across multiple systems. Distributed components can operate sequentially or in parallel.

In addition, the machine learning techniques described herein are readily interchangeable and combinable. Although certain example techniques have been described, many others exist and can be used in conjunction with aspects of the present disclosure.

A brief overview of example machine-learned models and associated techniques has been provided by the present disclosure. For additional details, readers should review the following references: Machine Learning A Probabilistic Perspective (Murphy); Rules of Machine Learning: Best Practices for ML Engineering (Zinkevich); Deep Learning (Goodfellow); Reinforcement Learning: An Introduction (Sutton); and Artificial Intelligence: A Modern Approach (Norvig).

Orthopedic surgery can involve implanting one or more implant components into a patient to repair or replace a damaged or diseased joint of the patient. For example, orthopedic shoulder surgery may involve attaching a first implant component to a glenoid cavity of a patient and attaching a second implant component to a humerus of the patient. It is important for surgeons to select the correct implant components and the plan the surgery properly when planning an orthopedic surgery. Some selected implant components and some planned procedures, involving positioning, angles, etc., may limit patient range of motion, cause bone fractures, or loosen and detach from patients' bones.

Over time and use, the implant may not function in the desired way or the patient condition may worsen in a way that makes the implant not function in the desired way. After the operational duration of the implant, additional corrective actions (e.g., surgery, such as revision surgery, or physical therapy) may be needed to alleviate symptoms of the patient condition.

An operational duration of implant may refer to information (e.g., a prediction) indicative of how long the implant will operate before additional corrective actions are needed. As one example, the information indicative of the operational duration of an implant may include information indicative of a likelihood that the implant will serve a function of the implant for a certain amount of time. For example, the operational duration of an implant may be information indicating that there is a 95% likelihood that the implant will serve its function for 10 years (e.g., for a group of patients who have the implant at 10 years, 95% of the patients still have the implant). As another example, the operational duration of an implant may be information indicative of likelihood that the implant will serve its function over a period of years (e.g., 99% likelihood that the implant will serve its function for 2 years, 99% likelihood that the implant will serve its function for 5 years, 95% likelihood that the implant will serve its function for 10 years, 90% likelihood that the implant will serve its function for 15 years, and so forth). As an example, the operational duration of the implant may be a histogram showing probability of duration for certain periods.

There may be various ways in which to qualify whether the implant will serve its function for a certain amount of time (e.g., whether there is efficacious or effective functioning of the implant). Example ways to determine the efficacious or effective function include determination of range of motion, tolerable or no pain, little to no dislocation, no implant breakage, and no infection.

The operational duration of an implant may be represented in other ways. As a few examples, the operational duration may be a particular time duration or range or classification (e.g., short, medium, long), with a likelihood or confidence of different durations. As described in more detail below, in some examples, rather than or in addition to a predicted duration for a particular selected implant, there may be a comparative ranking of other suitable implants by duration.

In some examples, the operational duration may be for the implant. However, there may be various factors, beyond just the size and shape of the implant, that may impact the operational duration such as an overall surgical plan that includes the implant along with positioning (medialization, lateralization, angle, etc.) of the implant.

Implanting some selected implant components with a certain designed surgical procedure may require the patient to undergo additional corrective actions earlier than necessary. For instance, the operational duration of a first implant, given the implant characteristics of the first implant, surgical procedure, and the patient characteristics of the patient, may be longer than the operational duration of a second implant, given the implant characteristics of the second implant, surgical procedure, and the patient characteristics of the patient. In this example, if a surgeon were to implant the second implant, the patient may require corrective actions earlier than if the surgeon were to implant the first implant. Without knowledge of the operational duration, in some cases, the surgeon may recommend the patient take corrective action earlier than necessary to ensure that the implant does not go past its operational duration.

However, taking corrective action, especially earlier than necessary, may be undesirable. Corrective action by the patient may increase cost to the patient and require the patient to undergo surgery, which increases the chances of infection, further damage to the bone or surrounding bone or tissue, and the like.

While ensuring that the implant selected for implantation has the longest operational duration (or an acceptable operational duration above a threshold) may be important, there may be other factors that impact which implant a surgeon is to use. As one example, a first implant may have a longer operational duration than a second implant if implanted in a particular patient. However, to implant the first implant, the surgeon may need to perform a surgical procedure that may not be appropriate for the patient, or the surgeon may be less experienced or skilled on that procedure, so the type of procedure can be balanced against the duration. For instance, the selected surgical procedure may last longer than would be advisable for the patient.

This disclosure describes example techniques performed by a computing system to generate information indicative of the operational duration of an implant. The surgeon may then utilize the information indicative of the operational duration of the implant to determine which implant to use prior to the surgery or during the surgery.

For example, the computing system may utilize a machine-learned model to determine the information indicative of the operational duration of the implant. The machine-learned model is a computer-implemented tool that may analyze input data (e.g., patient characteristics and implant characteristics), utilizing computational processes of the computing system in a manner that extends beyond just know-how of the surgeon to generate an output of the information indicative of the operational duration of the implant. The surgeon skill and experience may be additional examples of input data for the machine-learned model. Some additional examples of the input data include data from publications showing a survival rate of implants for a specific group of patients and a published revision rate for a selected implant range.

The computing system may apply model parameters of the machine-learned model to the input data (e.g., categorize the input data based on the model parameters or generally perform a machine learning algorithm using the model parameters on the input data). The result of applying the model parameters of the machine-learned model may be the information indicative of the operational duration of the implant.

The computing system may generate the model parameters of the machine-learned model based on a machine learning dataset. Examples of the machine learning dataset include one or more of pre-operative scans of a set of patients, patient characteristics of the set of patients, information indicative of previous surgical plans (e.g., surgical procedures) using different implants, information indicative of surgical results, and surgeon characteristics.

For example, the computing system may receive pre-operational or intra-operational data for a large number of cases. The pre-operational or intra-operational data may include information indicative of a type of surgery, scans of patient anatomy, patient information such as age, gender, diseases, smoker or not, fatty infiltration at bony region, etc., and implant characteristics such as dimension (e.g., size and shape), manufacturing company, stemmed or stemless configuration, stem size if stemmed, implant for anatomical or reverse implantation, etc. The computing system may also receive post-operational data for the large number of cases. The post-operational data may be information indicative of surgical results such as length of surgery, success or failure of proper implantation, infection rate, length of time before further corrective action was taken post implant, etc.

Additional examples of machine learning datasets may be data from patients that have had the implant implanted, and their results from the implantation. For example, after a patient is implanted with an implant, the patient may be periodically questioned about the comfort of the implant and a physician may also periodically determine movement capability of the patient. As one example, the patient may be asked questions like whether there is pain in certain body parts (e.g., shoulder). The patient may be asked questions such as whether their day-to-day life is impacted (e.g., in their daily living, in their leisure or recreational activity, during sleep, and how high they can move their arm without pain). The physician may determine the forward flexion, abduction, external rotation, and internal rotation of the patient. The physician may also determine how much weight the patient can pull.

All of these replies may be associated with a numerical score that is scaled to determine a composite score for the patient. This composite score may be referred to as a “constant score.” The composite score may be indicative of the success of the implantation. In some example, the composite score, or one or more of the numerical scores used to generate the composite score, may be machine learning datasets for training the machine-learned model. For example, each of the numerical scores used to generate the composite score may be indicative of how comfortable the patient is with the implantation, meaning that there is a lesser likelihood of needing corrective surgery soon. Utilizing the score information (e.g., scores used to generate composite score or composite score itself) from a plurality of patients that have been previously implanted may be helpful in determining an implant that is appropriate for the current patient.

The computing system may train (e.g., in a supervised or unsupervised manner) the machine-learned model using the pre-operational or intra-operational data as known inputs and the post-operational data as known outputs. The result of the training of the machine-learned model may be the model parameters. The computing system may periodically update the model parameters based on pre-operational or intra-operational and post-operational data of implant surgeries that are subsequently performed.

With the model parameters, the machine-learned model may be configured to generate information indicative of an operational duration of an implant. In some examples, the machine-learned model may be configured to generate information indicative of respective operational durations of a plurality of implants. The surgeon may then select one of the implants based on the information indicative of the respective operational durations.

For example, the model parameters may define operations that the computing system, executing the machine-learned model, is to perform. The inputs to the machine-learned model may be patient characteristics such as age, gender, diseases, smoker or not, and bone status (e.g., fatty infiltration, fracture, arthritic, etc.), as a few non-limiting examples. Additional inputs to the machine-learned model may be implant characteristics such as type of implant (e.g., anatomical or reversed, stemmed or stemless, etc.) and parameters of the implant (e.g., stem size, polyethylene (PE) insert thickness, etc.). As more examples, inputs to the machine-learned model may be information of the surgical skill/experience of the surgeon. The machine-learned model receives these examples of input data and groups, classifies, or generally performs the operation of a machine learning algorithm using the model parameters to determine operational duration of the implants.

Accordingly, in one or more examples, in predicting operational duration, the machine-learned model may utilize, as inputs, characteristics of an implant such as size, shape, angle, surgical positioning, and material. The machine-learned model may also utilize, as inputs, parameters such as orthopedic measurements obtained from CT image segmentation of the patient's joint, patient information, such as age, gender, shoulder classification (i.e., type of shoulder problem ranging from cuff tear to osteoarthritis). In some examples, the machine-learned model may also utilize, as input, physician information, such as preferences or experience/skill level/preferred implants.

The output from the machine-learned model may be the operational duration of the implant. For instance, the operational duration may be a particular time duration or range of classification (e.g., short, medium, long). The particular duration or range of classification may be associated with a likelihood or confidence for different durations. For instance, there is a 95% likelihood that implant serves its function after 10 years. As described in more detail elsewhere in this disclosure, in addition to a predicted duration for a particular implant, the machine-learned model may perform its example operations for a plurality of implants and may provide a comparative ranking of other suitable implants by duration.

Also, in some examples, the operational duration may be based on a particular surgical procedure (e.g., surgical plan). The operative technique (e.g., surgical procedure) may be different for different types of implants. The number of steps needed for the surgical procedure may be correlated with the operational duration of the implant. The example techniques may account for the number of steps needed for the surgical procedure in determining the operational duration of the implant.

In some examples, for the same implant and same patient, the machine-learned model may determine operational durations for the implant for different surgical procedures. For instance, for a first implant, the machine-learned model may generate operational duration information for a plurality of time periods (e.g., 2, 5, 10, and 15 years), and for each time period, the machine-learned model may generate operational duration information for different surgical procedures. The machine-learned model may repeat the process for a second implant, and so forth. In some examples, machine-learned model may perform a subset of the example operations (e.g., generate duration information for only one time period). In general, machine-learned model may determine different types of operational duration information using the techniques described in this disclosure, and machine-learned model may determine all or a subset of the examples of the operational duration information.

As one example way in which the machine-learned model may operate, the machine-learned model, using the model parameters, may determine a classification based on the input data. The classification may be associated with a particular value for the operational duration. For instance, the model parameters may include a plurality of clusters for each of a plurality of implants, where each cluster represents a multi-dimensional range of values for the input data. Each of the clusters may be associated with a particular operational duration for respective implants. The machine-learned model may be configured to determine a cluster based on the input data and then determine the operational duration of the implant based on the determined cluster.

As another example way in which the machine-learned model may operate, the machine-learned model, using the model parameters, may scale a baseline operational duration value based on numerical representations of the input data, where the amount by which the machine-learned model scales the baseline operational duration value is based on the model parameters. For example, the baseline operational duration value for a particular implant may be 90% likelihood of serving its function for 10 years. Based on the input data and the model parameters, the machine-learned model may scale the 90% down to 80% or scale the 90% to 95%.

In some examples, the machine-learned model may be further configured to compare operational duration of different implants based on patient characteristics and output a recommendation for an implant. For instance, the machine-learned model may further analyze, based on the model parameters, factors such as length of operation needed to implant, cost of implantation, risk of infection during the operation, quality of life expectancy post-implant (e.g., such as based on a determination of the composite score or scores used to form the composite score), and other such factor. For each of the implants, the machine-learned model may generate a feasibility score for each of the implant. The feasibility score may indicative of how beneficial the implant would be to the patient. The machine-learned model may compare (e.g., as a weighted comparison) the feasibility score and the operational duration of each implant with other implants and output a particular implant as the recommended implant based on the comparison.

As described herein, FIGS. 9 through 13 are conceptual diagrams illustrating aspects of example machine-learning models. For ease of understanding, the example techniques are described with respect to FIGS. 9 through 13. As one example, machine-learned model 902 of FIG. 9 is an example of a machine-learned model configured to perform example techniques described in this disclosure. As described in this disclosure, machine-learned model 720 of FIG. 7 is an example of machine-learned model 902. Any one or a combination of computing device 1002 (FIG. 10), server system 1104 (FIG. 11), and client computing device 1202 (FIG. 12) may be examples of a computing system configured to execute machine-learned model 902. In one or more examples, machine-learned model 902 may be generated with model trainer 1208 (FIG. 12) using example techniques described with respect to FIG. 13.

For instance, machine-learned model 902 may be configured to determine and output information indicative of an operational duration of an implant based on patient characteristics of a patient and implant characteristics of an implant, and in some examples, based on surgical procedure and/or surgeon experience. A surgeon may receive the information indicative of the operational duration and select an implant to use based on the information indicative of the operational duration. As described in more detail, machine-learned model 902 may generate information indicative of operational durations of a plurality of implants, and the surgeon may select an implant from the plurality of implants based on the information indicative of the operational durations.

A computing system, applying machine-learned model 902, may be configured to obtain patient characteristics of a patient and obtain implant characteristics of an implant. The patient characteristics may include one or more age of the patient, gender of the patient, diseases of the patient, whether the patient is a smoker, and fatty infiltration in tissue adjacent target bone where the implant is to be implanted. The patient characteristic may also include information such as type of disease the patient is experiencing (e.g., if shoulder problem, the problem may be for cuff tear or osteoarthritis). The implant characteristics may include one or more of a type of implant and dimensions of the implant. For example, the implant characteristics may include information indicating whether the implant is for an anatomical or reversed implant procedure, whether the implant is stemmed or stemless, and the like. The implant characteristics may also include information indicating parameters of the implant such as stem size, polyethylene (PE) insert thickness, and the like. In some examples, the computing system, applying machine-learned model 902, may also be configured to obtain information of the surgical procedure (e.g., plan), including positioning of the implant. For instance, the surgical procedure may include information such as medialization and lateralization angles. Also, the surgical procedure) may be different for different types of implants. The number of steps needed for the surgical procedure may be correlated with the operational duration of the implant. Machine-learned model 902 may account for the number of steps needed for the surgical procedure in determining the operational duration of the implant.

The computing system, applying machine-learned model 902, may be configured to determine information indicative of an operational duration of the implant based on the patient characteristics and the implant characteristics. For example, machine-learned model 902 may determine information indicative of a likelihood that the implant will serve a function of the implant for a certain amount of time. As one example, machine-learned model 902 may determine information such as there being a 90% likelihood that the implant will serve its function for 10 years. In this example, there is a 10% likelihood that the patient will need revision or some other form of corrective action in the first 10 years.

As another example, the operational duration of an implant may be information indicative of a likelihood that the implant will serve its function over a period of years (e.g., a 99% likelihood that the implant will serve its function for 2 years, a 99% likelihood that the implant will serve its function for 5 years, a 95% likelihood that the implant will serve its function for 10 years, a 90% likelihood that the implant will serve its function for 15 years, and so forth). As an example, the operational duration of the implant may be a histogram showing a probability of duration for certain periods.

In some examples, the operational duration information may be relative information such as the operation duration is short, medium, or long. The operational duration information may be associated with a likelihood or confidence value (e.g., very likely that the operational duration of implant is at least short term).

The operational duration information may be for an implant, and in some examples, for a specific surgical procedure. In some examples, the surgeon may utilize the operational duration information to assist with surgical planning (e.g., select the surgical procedure that provides the longest operational duration (or at least above a threshold duration) balanced with the highest likelihood and other factors such as a length of surgical procedure).

The computing system, applying machine-learned model 902, may be configured to output the information indicative of the operational duration of the implant (e.g., which may be a plurality of cooperative components such as a humeral head with stem and a glenoid plate). A health professional (e.g., one or more surgeon, nurse, clinician, etc.) may utilize the information indicative of the operational duration of the implant to select an implant to use for the surgery, as well as a surgical plan in some examples.

As an example, the computing system may be virtual planning system 701 of FIG. 7, and one or more storage devices 714 of virtual planning system 701 store one or more machine-learned models 720 (e.g., object code of machine-learned models 720 that is executed by one or more processors 702 of virtual planning system 701). As described in this disclosure, one example of machine-learned models 720 is machine-learned model 902. One or more storage devices 714 store surgery planning module 718 (e.g., object code of surgery planning module 718 that is executed by one or more processors 702).

A health professional (e.g., surgeon, nurse, clinician, etc.), as part of the pre-operative planning or intra-operative planning, may cause one or more processors 702 to execute surgery planning module 718 using one or more input devices 710. The health professional may enter, using one or more input devices 710, the patient characteristics and the implant characteristics. In some examples, a range of implant characteristics may be recommended by the system based on automated planning using segmentation and image processing. In this way, the computing system (e.g., virtual planning system 701) may obtain the patient characteristics and the implant characteristics. The health professional may also enter information of the surgeon (e.g., surgical experience, preferences, etc.).

Executing surgery planning module 718 may cause one or more processors 702 to perform operations defined by one or more machine-learned models 720, including operations defined by machine-learned module 902. For example, one or more processors 702 may determine information indicative of an operational duration of the implant based on the patient characteristics and the implant characteristics (e.g., information indicative of a likelihood that the implant will serve a function of the implant for a certain amount of time).

One or more output devices 712 of virtual planning system 701 may be configured to output information indicative of the operational duration of the implant. For example, in some examples, one or more display devices 708 may be part of output devices 712, and one or more display devices 708 may be configured to present information indicative of the operational duration of the implant. In some examples, one or more output devices 712 may include one or more communication devices 706. One or more communication devices 706 may output the information indicative of the operational duration of the implant to one or more visualization devices, such as visualization device 213. In such examples, visualization device 213 may be configured to display the information indicative of the operational duration of the implant (e.g., likelihood and duration values, likelihood histograms, ranking system, etc.).

The above example with respect to virtual planning system 701 is provided as one example and should not be considered limiting. For instance, other examples of a computing system such as computing device 1002 (FIG. 10) and client computing device 1202 (FIG. 12) may be configured to operate in a substantially similar manner.

In some examples, server system 1104 of FIG. 11 may be an example of a computing device. In such examples, server system 1104 may obtain the patient characteristics and the implant characteristics based on information provided by a health professional using client computing device 1102 of FIG. 11. Processing devices of server system 1104 may perform the operations defined by machine-learned model 902 (which is an example of machine-learned models 720 of FIG. 7). Server system 1104 may output the information indicative of the operational duration of the implant back to client computing device 1102. Client computing device 1102 may then display information indicative of the operational duration of the implant or may further output the information indicative of the operational duration of the implant to visualization device 213.

As described above, a computing system, using machine-learned model 902, may be configured to determine information indicative of the operational duration of the implant. The information indicative of the operational duration of the implant may be information indicative of how long before corrective action may be needed. As one example, the operational duration of the implant may indicate a likelihood of the implant serving its function (e.g., restoring joint mobility, paint reduction, no dislocation, no implant fracture, etc.) for a certain amount of time. Examples of corrective action may include revision surgery (which may involve removal of implant and implantation of a different type of implant with a different surgical procedure), replacement of the implant (e.g., removing and replacing with similar implant), physical therapy to accommodate for the reduction in functionality of the implant, and the like.

As described above, there may be various ways in which to qualify whether the implant will serve its function for a certain amount of time such as based on efficacious or effective function. Examples ways to determine efficacious or effective function include determination of range of motion, tolerable or no pain, little to no dislocation, no implant breakage, and no infection.

For example, effective function of a joint may mean that a pain score for the patient is below a certain level. As another example, effective function may mean that an activity score associated with impact on day-to-day life is within a particular range. As another example, effective function may mean that the forward flexion score is greater than a particular angle and the abduction score is greater than a particular angle. As another example, a rotation score indicative of external rotation and internal rotation may be indicative of effective function. A power score indicative of an amount of weight that the patient can pull may be indicative of effective function. These various scores may be combined together to form a composite score, also referred to as a constant score.

In one or more examples, the various scores or the composite score may be used as part of the training set for training the machine-learned model 902. For instance, utilizing the various scores for patients that have already had the implant may be predictive for the duration of the implant in a current patient, such as being indicative of whether the current patient is predicted to find the implant satisfactory, and hence, lower likelihood of needing a replacement.

In some examples, machine-learned model 902 of the computing system may receive the patient characteristics and the implant characteristics and apply model parameters of the machine-learned model to the patient characteristics and the implant characteristics, as described in this disclosure with respect to FIG. 9. Machine-learned model 902 may determine the information indicative of the operational duration based on the application of the model parameters of the machine-learned model.

There may be various ways in which machine-learned model 902 may apply the model parameters to determine the information indicative of the operational duration of the implant. As one example, machine-learned model 902, using the model parameters, may determine a classification based on the patient characteristics and the implant characteristics. The classification may be associated with a particular value for the operational duration. For instance, the model parameters may include a plurality of clusters for each of a plurality of implants, where each cluster represents a multi-dimensional range of values for the patient characteristics and the implant characteristics. Each of the clusters may be associated with a particular operational duration for respective implants. Machine-learned model 902 may be configured to determine a cluster based on the patient characteristics and the implant characteristics and then determine the operational duration of the implant based on the determined cluster.

As another example, machine-learned model 902, using the model parameters, may scale a baseline operational duration value based on numerical representations of the patient characteristics and the implant characteristics, where the amount by which machine-learned model 902 scales the baseline operational duration value is based on the model parameters. For example, the baseline operational duration value for a particular implant may be 90% likelihood of serving its function for 10 years. Based on the input data and the model parameters, machine-learned model 902 may scale the 90% down to 80% or scale the 90% to 95%.

As one or more example, machine-learned model 902 may utilize the model parameters generated from random forest machine-learning techniques. As another example, the model parameters may be for a neural network.

In the above examples, a computing system, using machine-learned model 902, may determine an operational duration for an implant. In some examples, the computing system, using machine-learned model 902, may determine respective operational durations for a plurality of implants. For instance, machine-learned model 902 may receive implant characteristics for each of a plurality of implants. For each implant of the plurality of implants, the computing system, using machine-learned model 902, may determine an operational duration. In some examples, for each implant and for each of a plurality of different surgical procedures (e.g., as input by the health professional or as automatically determined), machine-learned model 902 may determine an operational duration. The health professional may review the operational durations for each of the plurality of implants and select one of the implants. In examples where a surgical procedure is also a factor used in determining operational durations, the health professional may select one of the implants further based on the surgical procedure and operational duration or vice-versa (i.e., select surgical procedure based on operational duration for implant).

In some examples, the computing system, using machine-learned model 902, may compare the operational durations for each of the plurality of implants and select an implant of the plurality of implants based on the comparison. For instance, the computing system, using machine-learned model 902, may compare the likelihood values for each of the operational durations for each of the implants and select the implant having the highest likelihood value (e.g., implant having the highest likelihood of serving its function for a certain amount of time). In some examples, rather than relying only on the highest likelihood value, machine-learned model 902 may select the implant having a likelihood value that meets a threshold.

The computing device may then output information indicative of the operational duration of the selected implant, as the recommended implant. The health professional may then choose to accept the recommendation of the recommend implant or reject the recommendation.

In some examples, the computing system, using machine-learned model 902, may rank the implants based on the comparison. For instance, the computing system may output, for display, information indicative of the operational duration of each of the implants, but in an order most recommended to least recommended. The health professional may then review the ranking to select the implant.

The operational duration of the implant may be one factor that machine-learned model 902 may utilize in recommending or ranking the implants. In some examples, the computing system, using machine-learned model 902, may be configured to compare the information indicative of the operational duration of each of the plurality of implants based on patient characteristics and/or surgical procedure.

For certain patients, implanting the implant with the longest operational duration may not be ideal. As an example, the surgical procedure for implanting the implant with the longest operational duration may not be safe for the patient given the patient characteristics. As another example, the implant with the longest operational duration may not be ideal for a patient given his or her life expectancy. As another example, implantation of the implant with the longest operational duration may result in lower quality of life as compared to another implant (e.g., more limited range of mobility as compared to another implant). There may be various other factors that impact which implant to select.

In some examples, machine-learned model 902 may utilize information of patient characteristics to further refine the determination of which implant to recommend. For example, machine-learned model 902 may determine a feasibility score for each implant. The feasibility score may be indicative of how beneficial the implant is for the patient. The feasibility score may be based on a combination of a plurality of patient factors. Examples of the plurality of patient factors may include two or more of length of surgery, risk of infection, mobility post-implant, recovery from surgery, price of implant, and the like. For instance, the feasibility score may be based on a prediction of the composite score or the various scores used to generate the composite score such as one or more of the pain score, activity score, forward flexion score, abduction score, a rotation score indicative of external rotation and internal rotation, and a power score may be indicative of effective function.

The computing system, using machine-learned model 902, may be configured to determine a value for one or more of the patient factors and determine a feasibility score based on a combination (e.g., weighted average) of the values of the patient factors. The computing system may then output the feasibility score for the implant in addition to the operational duration. In some examples, rather than determining a single feasibility score, machine-learned model 902 may be configured to determine values for the one or more patient factors. In such examples, the values for the one or more patient factors may be each considered as examples of a feasibility score. That is, in some examples, the feasibility score refers to a single feasibility score based on a combination of values for patient factors, and in some examples, each of the values of the patient factors may be considered as a feasibility score.

Machine-learned model 902 may be configured to output information indicative of an operational duration for each of the implants (and possibly for each surgical procedure) and information indicative of the one or more feasibility scores. The health professional may then select a particular implant based on the operational duration and the feasibility score. In some examples, machine-learned model 902 may be configured to recommend a particular implant based on the operational duration and respective feasibility scores for the plurality of implants. For example, the computing system, using machine-learned model 902, may be configured to select one of the plurality of implants based on the comparison of the information indicative of the operational duration of each of the plurality of implants and the comparison of the one or more feasibility scores.

As an example, a first patient factor may be how long the surgery would take, a second patient factor may be the chances of infection, a third patient factor may be a range of mobility (or more generally, one of the example scores described above), and a fourth patient factor may be length of recovery. Machine-learned model 902 may determine how long the surgery would take to implant a first implant (e.g., a value for a first patient factor), the chances of infection (e.g., a value for a second patient factor), the range of mobility (e.g., a value for a third patient factor), and a length of recovery time (e.g., a value for a fourth patient factor). Based on the values for the first, second, third, and fourth patient factors, machine-learned model 902 may determine a feasibility score for the first implant. Machine-learned model 902 may repeat these operations for the plurality of implants.

Machine-learned model 902 may utilize the operational duration and feasibility score as factors in determining which implant to recommend. For example, machine-learned model 902 may weigh the operational duration information and the feasibility score to recommend a particular implant, and in some examples, accounting for the surgical procedure. For example, if the implant having the highest likelihood of serving its function for a certain period of time also has the highest feasibility score, then machine-learned model 902 may recommend that implant. However, if an implant has a relatively high likelihood of serving its function for a certain period of time but has a relatively low feasibility score, machine-learned model 902 may be configured to recommend another implant with a lower likelihood of serving its function for the certain period of time but with a higher feasibility score. By how much the operational duration and feasibility scores are weighted may be a factor of design choice and may be different for different types of surgeries and different patients.

In one or more examples, machine-learned model 902 may be trained using model trainer 1208 (FIG. 12), such as by using the techniques described with respect to FIG. 13, as one example. For example, model trainer 1208 may be configured to train machine-learned model 902 based on a machine learning dataset. The machine learning dataset may be information from surgeries performed on many different patients. The machine learning dataset may include pre-operative scans of a plurality of patients (e.g., such as information derived from segmentation of these scans), information indicative of surgical plans used for the surgery on the plurality of patients, information taken from follow up visits (e.g., such as the scores for generating the composite score), and patient's information such as age, weight, smoker or not, types of diseases, and the like. Examples of the information indicative of surgical plans include delto-pectoral approach or supero-lateral approach, or information such as type of glenoid, as a few examples.

As one example, the machine learning dataset may include information such as operational duration for a plurality of implants that were previously implanted in different patients. The machine learning dataset may include information such as length of surgery, mobility of patient after surgery, whether there was an infection or not, the length of recovery, and the like.

For example, training data 1302 may include information such as patient ages, weight, height, smoker or not, types of diseases, etc., types of implants, and the like. Training data 1302 may include surgical experience. Example input data 1304 may be the actual information about the patient ages, weight, height, smoker or not, types of diseases, and the implant used in patients. Some additional examples of the input data 1304 include data from publications showing survival rate of implants for specific group of patients and published revision rate for a selected implant range. Output data 1308 may be the operational duration of the implants used for the patients that make up the example input data 1304. Output data 1308 may also include information such as the length of surgery, mobility of patient after surgery, whether there was an infection or not, the length of recovery, actual surgical procedure used and the like.

Objective function 1310 may utilize the example input data 1304 and the output data 1308 to determine model parameters for machine-learned model 902. For example, the model parameters may be weights and biases, or other example parameters described with respect to FIGS. 9-13. The result of the training may be that machine-learned model 902 is configured with model parameters that can be used to determine operational duration and, optionally, feasibility score(s) for implants.

In this way, this disclosure describes example techniques utilizing computational processes for selecting an implant for implantation. The example techniques described in this disclosure are based on machine learning datasets that may be extremely vast with more information that could be accessed or processed by a surgeon without access to the computing system that uses machine-learned model 902. For instance, surgeons with limited experience may not have sufficient know-how for how to accurately determine which implant, from among multiple implants, to use, given an objective for prolonged operation and delayed need for revision surgery. Even experienced surgeons may not have access and may not be able to comprehend the vast information available that is used to train machine-learned model 902.

For example, even if a surgeon were to access and review the information from the dataset, the surgeon may still not be able to, given the vast amount of information, to construct a surgical technique, e.g., including implant(s) selection and positioning, that accurately accounts for all the different patient information and implant characteristics. However, machine-learned model 902 may be configured to utilize the vast amount of information as a way to determine operational duration of an implant and select an implant, in some examples, as the recommended implant. Moreover, using machine-learned model 902 may allow for a scalable, extensible computing system that can be updated with new information periodically to create a better version of machine-learned model 902. A surgeon may not have the ability to update his/her understanding of what the operational duration or what the recommended implant should be, much less update as quickly as machine-learned model 902 can be updated.

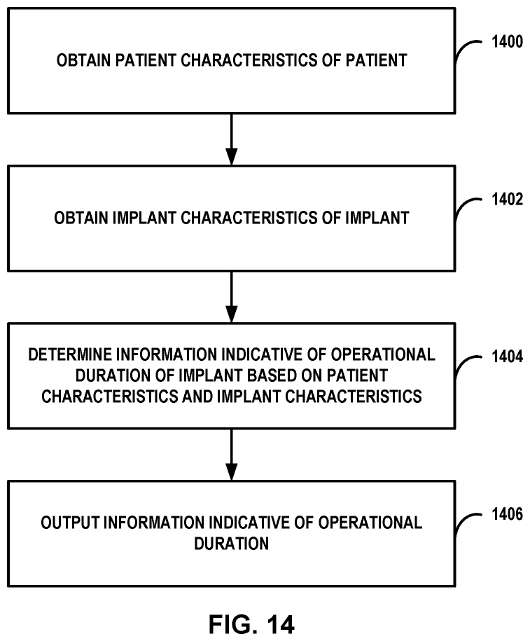

FIG. 14 is a flowchart illustrating an example method of determining information indicative of an operational duration of an implant. For ease of description, the example of FIG. 14 is described with respect to FIG. 7 and machine-learned model 902 of FIG. 9, which is an example of machine-learned model 720 of FIG. 7. However, the example techniques are not so limited.

As illustrated in FIG. 7, storage device(s) 714 stores machine-learned model(s) 720, an example of which is machine-learned model 902. One or more processors 702 may access and execute machine-learned model 902 to perform the example techniques described in this disclosure. One or more storage devices 714 and one or more processors 702 may be part of the same device or may be distributed across multiple devices. For instance, virtual planning system 701 is an example of a computing system configured to perform the example techniques described in this disclosure.

One or more processors 702 (e.g., using machine-learned model 902) may obtain patient characteristics of a patient (1400). For example, the patient characteristics include one or more age of the patient, gender of the patient, diseases of the patient, whether the patient is a smoker, and fatty infiltration of tissue adjacent a target bone where the implant is to be implanted. A health professional may provide information of the patient characteristics using input devices 710 as an example.

One or more processors 702 may obtain implant characteristics of an implant (1402). In some examples, the implant characteristics of the implant include one or more of a type of implant and dimensions of the implant (e.g., for reverse or anatomical, stemmed or stemless, etc.). A health professional may provide information of the implant characteristics using input devices 710 as an example. In some examples, one or more processors 702 may obtain implant characteristics of a plurality of implants to perform the example techniques on a plurality of implants.

One or more processors 702 may determine information indicative of an operational duration of the implant based on the patient characteristics and the implant characteristics (1404). As one example, one or more processors 702 may determine information indicative of the likelihood that the implant will serve a function of the implant for a certain amount of time. In examples where there is a plurality of implants, one or more processors 702 may determine information indicative of an operational duration for one or more (including all) of the implants.

There may be various ways in which one or more processors 702 determine the information indicative of the of the operational duration of the implant. As one example, one or more processors may receive, with machine-learned model 902, the patient characteristics and the implant characteristics, apply model parameters of machine-learned model 902 to the patient characteristics and the implant characteristics, and determine the information indicative of the operational duration based on the application of the model parameters of machine-learned model 902.

In some examples, the model parameters of machine-learned model 902 are generated based on a machine learning dataset. For example, the machine learning dataset includes one or more of pre-operative scans of a set of patients, patient characteristics of the set of patients, information indicative of previous surgical plans using the same or different implants, and information indicative of surgical results.

One or more output devices 712 may be configured to output the information indicative of the operational duration of the implant (1406). For example, one or more processors 702 may output the information indicative of the operational duration of the implant to one or more output devices 712. One or more output devices 712 may display the operational duration of the implant (e.g., in examples where display device 708 is part of output devices 712). In some examples, one or more output devices 712 may output the information indicative of the operational duration of the implant to another device, such as visualization device 213, for display.

In examples where there is a plurality of implants, output devices 712 or visualization device 213 may output information indicative of the operational duration for the plurality of implants. However, in some examples, one or more processors 702 may compare the information indicative of the operation duration for the plurality of implants to select a recommendation of the implant.

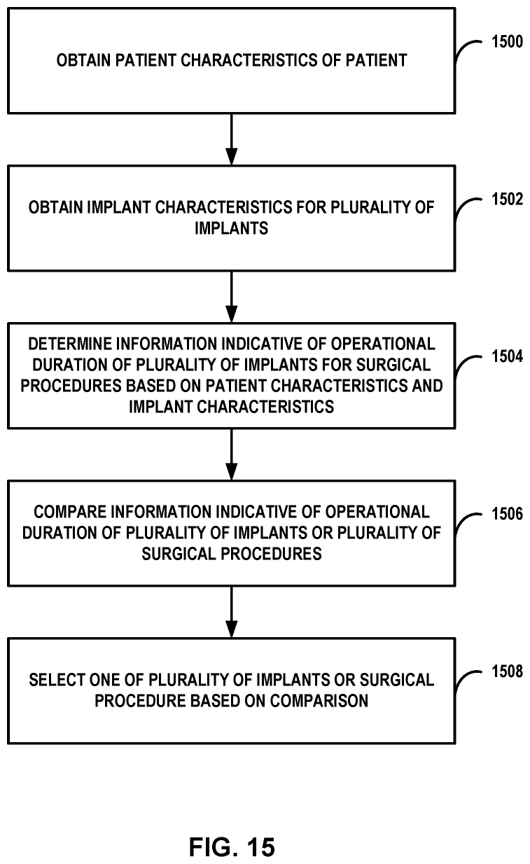

FIG. 15 is a flowchart illustrating an example method of selecting an implant. Similar to FIG. 14, one or more processors 702 may obtain patient characteristics of a patient (1500). One or more processors 702 may obtain implant characteristics for a plurality of implants (1502). For example, the implant described in FIG. 14 may be a first implant and one or more processors 702 may obtain the implant characteristics for a plurality of implants, including the first implant.

In the example illustrated in FIG. 15, one or more processors 702 may determine information indicative of operational duration of a plurality of implants for surgical procedures based on patient characteristics and implant characteristics (1504). For example, one or more processors 702 may determine an operational duration for the first implant, an operational duration for a second implant, and so forth. In some examples, one or more processors 702 may determine an operational duration for a first surgical procedure for a first implant, for a second surgical procedure for the first implant, and so forth for the first implant. One or more processors 702 may repeat such operations for other implants. For example, one or more processors 702 may determine information indicative of the operational duration of the implant for a first surgical procedure, and determine information indicative of a plurality of operational durations for the implant for a plurality of surgical procedures. Rather than performing such operations for a plurality implants, in some examples, one or more processors 702 may perform such operations only for the first implant.

In some examples, one or more processors 702, with output devices 712, may output the information indicative of the operational duration of the plurality of implants and/or information indicative of the surgical procedures. For example, output devices 712 may output information such as short, medium, long with likelihood or confidence values for the operational duration, a value indicative of a likelihood over a period of time, or a histogram of likelihood values at certain time periods, as a few examples. In some examples, output devices 712 may output information such as the surgical procedure associated with achieving the operational duration (e.g., implant location, medialization, lateralization angles, etc.).

However, in some examples, rather than or in addition to outputting the information indicative of the operational duration of the plurality of implants, one or more processors 702 may compare the information indicative of the operational duration of the plurality implants or plurality of surgical procedures (1506). For example, one or more processors 702 may compare the values of each implant indicating the likelihood that the implant will serve its function (e.g., provide mobility while remaining implanted with minimal pain or discomfort) for a certain amount of time.

One or more processors 702 may select one of the plurality of implants or surgical procedure based on the comparison (1508). For example, one or more processors 702 may select the implant with the highest likelihood of serving its function for the certain amount of time. In some examples, output devices 712 may output information indicative of the selected implant.

In some examples, as a result of the comparison, one or more processors 702 may rank each of the implants based on the operational duration. For example, one or more processors 702 may rank first the implant with the highest likelihood of serving its function for the certain amount of time, followed by the second implant with second highest likelihood, and so forth.

In some examples, as a result of the comparison, one or more processors 702 may rank each of the surgical procedures (e.g., which one takes least amount of time, which one is safest, etc.). One or more output devices 712 may be configured to output the ranked list or lists.

In the above example, one or more processors 702 may determine operational duration and rank implants or select an implant based on the operational duration. However, in some examples, one or more processors 702 may also determine one or more feasibility scores to rank implants or surgical procedure or select an implant or surgical procedure based on the operational duration and feasibility scores.

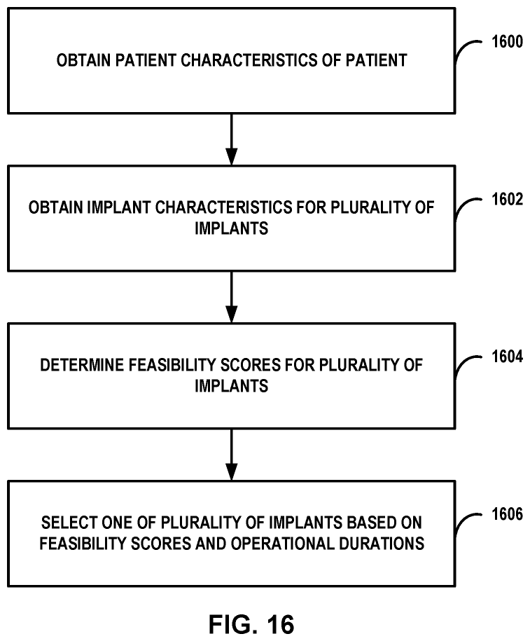

FIG. 16 is a flowchart illustrating another example method of selecting an implant. Similar to FIGS. 14 and 15, one or more processors 702 may obtain patient characteristics of a patient (1600) and obtain implant characteristics for plurality of implants (1602). One or more processors 702 may determine one or more feasibility scores for the plurality of implants, as described above (1604). For example, the feasibility score may be indicative of how beneficial the implant is for the patient. The feasibility score may be based on a combination of a plurality of patient factors. Examples of the plurality of patient factors include length of surgery, risk of infection, mobility post-implant, recovery from surgery, and the like (e.g., the composite score or one or more scores used to generate the composite score). One or more processors 702 may be configured to weight one or more of the plurality of patient factors differently and associated values to determine a feasibility score.

In one or more examples, output devices 712 may be configured to output a list of implants with their operational duration scores and feasibility scores. In some examples, output devices 712 may output a ranked list of the implants with their operational duration scores and feasibility scores.

In the example illustrated in FIG. 16, one or more processors 702 may be configured to select an implant from the plurality of implants based on the operational duration scores and feasibility scores (1606). For example, one or more processors 702 may be configured to weigh the operational duration score and the feasibility score based on patient characteristics to determine which implant should be recommended to the surgeon for implantation in the patient.

Orthopedic surgery can involve implanting one or more implant components into a patient to repair or replace a damaged or diseased joint of the patient. For example, an orthopedic shoulder surgery may involve attaching a first implant component to a glenoid cavity of a patient and attaching a second implant component to a humerus of the patient. The first and second implant components may cooperate with one another to replace the shoulder joint and restore motion and/or reduce discomfort. It is important for surgeons to select from properly designed implant components when planning an orthopedic surgery. Improperly selected or improperly designed implant components may limit patient range of motion, cause bone fractures, or loosen and detach from patients' bones or require more follow up visits.

In many cases, the implant that a surgeon selects need not necessarily be a patient specific implant. For example, an implant manufacturer may generate a plurality of different implants having different dimensions and shapes. The surgeon may select from one of these pre-manufactured implants as part of the pre-operative planning or, possibly, intra-operatively. For instance, rather than having an implant custom manufactured for a patient (e.g., based on pre-operative information of the patient or possibly intra-operatively with a 3D printer), the surgeon may select from one of the pre-manufactured implants. In some examples, it may be possible that the surgeon selects a particular implant, and then the implant is manufactured (e.g., such as where the manufacturer or hospital does not have the particular implant in stock). However, the implant manufacturing may be done without information of the specific patient who is to be implanted with the implant.

Although the implant may be not specific for a patient, the implant may be manufactured for a particular group of patients. For instance, the group of patients may be gender based, height based, obesity based, etc. As an example, the manufacturer may generate an implant that, while not specific to a particular patient, may be generally for obese patients, or male patients, or tall patients, etc.

In some examples, the implant may be manufactured based on specific patient information. For instance, as part of the pre-operative planning, the surgeon may determine patient dimensions (e.g., size of bone where implant is to be implanted) and patient characteristics (e.g., age, weight, sex, smoker or not, etc.). A manufacturer may then construct a patient specific implant.

In both examples (e.g., non-patient specific implant or patient specific implant), the implant manufacturing procedure should manufacture an implant that will be well-suited for implantation. For example, a surgeon should be able to implant with effort well within the range of normal surgical effort. If implanted in a competent manner, the implant should not cause any additional damage to the target bone, surrounding bone, or surrounding tissue. The implant should serve its function for a reasonable amount of time before the patient needs to take corrective actions (e.g., having implant replaced with same type of implant, having a reversed implant surgery, undergoing extensive physical therapy, etc.).

There may be technical problems in manufacturing implants to achieve the above example goals of the implant (e.g., reasonable implant effort, low amount of damage to bone or surrounding area, long functional time, etc.). For instance, an implant designer, which may be person or a machine, may have a limited knowledge base of how to design an implant that satisfies the various goals. With a human implant designer, the amount of knowledge needed to ensure that the implant satisfies the example goals would be too vast and a human implant designer, or even a team of implant designers, would not be able to know all of the information needed to design an implant that satisfies the example goals.

This disclosure describes example techniques of utilizing machine-learning techniques for practical applications of designing implants. For instance, a computing system utilizing a machine-learned model may be configured to perform the example techniques described in this disclosure, which a human designer or a team of human designers would not be able to perform. In some examples, it may be possible that a human designer or team of human designers can construct an example implant and input information of the implant into the machine-learned model. The machine-learned model, in this example, indicates whether the implant would be suitable or not.

The computing system may utilize a machine-learned model to determine the size and shape of an implant. The machine-learned model is a tool that may analyze input data (e.g., implant characteristics of an implant to be manufactured) utilizing computational processes of the computing system in a manner that extends beyond just know-how of a designer to generate an output of the information indicative of the dimensions of the implant (e.g., size and shape). As one example, the implant characteristics of the implant to be manufactured include information that the implant is for a type of surgery (e.g., anatomical or reversed), information that the implant is stemmed or stemless, information that the implant is for a type of patient condition (e.g., for fracture, for osteoporosis, etc.), information that the implant is for a particular bone (e.g., humerus, glenoid, etc.), and information of a press fit area (e.g., distal press fit or proximal press fit) of the implant (e.g., area around which bone is to grow to secure the implant in place). The following are some additional examples of implant characteristics: length of the stem in case of a revision stem, graft window for revision or fracture cases, locking screw to lock the stem in the humerus in case of revision, convertible prosthesis, monolithic or modular prosthesis, stem shape that mimics the internal shape of the humerus or not, etc.

The computing system may apply model parameters of the machine-learned model to the input data (e.g., categorize the input data based on the model parameters or generally perform a machine learning algorithm using the model parameters on the input data) and the result of applying the model parameters of the machine-learned model may be the information indicative of the dimensions of the implant. For instance, the machine-learned model may receive the implant characteristics. Implant characteristics may be information of a way in which the implant is to be used and not necessarily size and shape of the implant. However, information of the size and shape of the implant that the machine-learned model modifies may be possible. The machine-learned model may apply model parameters of the machine-learned model. The machine-learned model may determine the information indicative of the dimensions of the implant based on the applying of the model parameters of the machine-learned model.

The computing system may generate the model parameters of the machine-learned model based on a machine learning dataset. Examples of the machine learning dataset includes one or more of information indicative of clinical outcomes (e.g., information indicative of survival rate (how long the implant lasted), range of motion, pain level, etc.) for different types of implants and dimensions of available implants. For example, similar to the above description for determining operational duration, part of the information indicative of clinical outcomes may be composite scores or scores used to generate the composite score from patients that have had an implant implanted. For instance, a pain score associated with an implant may be indicative of a pain level for a patient. An activity score may be associated with impact on day-to-day life of the patient. The forward flexion score and the abduction score may be indicative of an amount of movement by the patient. A rotation score indicative of external rotation and internal rotation may indicate how well the patient can rotate his/her shoulder and arm. A power score may indicate how much weight the patient can move. These various scores may be combined together to form a composite score, also referred to as a constant score. Such score information may be indicative of how well an implant will function, and may help guide how an implant is to be constructed.

As another example, information indicative of clinical outcomes may include information available from articles and publications of clinical outcomes. Examples of information available from articles includes survival rate, range of motion, pain level, revision rate, dislocation rate, and infection rate. The above examples of information available from articles may be for each of a plurality of patient characteristics like mean age, gender, diseases, dominant arm, etc. The articles and publications may also include information collected directly from physicians performing procedures.

For example, the computing system may receive implant data for a large number of implants. The implant data may include information indicative of clinical outcomes of the various implants and implant 3D models. For instance, the implant data may include information of implants that were used in surgery (e.g., as trial or permanent) and what the outcome of the surgery was. Examples of the outcome of the surgery include information such as a length of time that the surgery took, how difficult the surgery was, how much damage there was to the bone and surrounding area, and how long the implant served its function, as a few examples. In addition, for each of the implants, the patient information may also be used as input, such as type of surgery for which the implant was used, type of bone on which the implant was affixed, bone characteristics (e.g., how much available bone there was), and other characteristics like patient disease.

The computing system may train (e.g., in a supervised or unsupervised manner) the machine-learned model using the implant data and patient characteristics as known inputs and the clinical outcomes as known outputs. The result of the training of the machine-learned model may be the model parameters. The computing system may periodically update the model parameters based on implant data and clinical outcomes generated subsequently. For example, the machine-learned model receives different implants and outcomes and uses the different implants and outcomes to pick the best ones (best size/shape) for a recommended design.

With the model parameters, the machine-learned model may be configured to generate information indicative of dimensions (e.g., size and shape) of an implant that is be designed and manufactured. In some examples, with the dimensions of the implant a manufacturer may manufacture the implant.

For example, the model parameters may define operations that the computing system, executing the machine-learned model, is to perform. The inputs to the machine-learned model may be implant characteristics of an implant to be manufactured such as information that the implant is for a type of surgery, information that the implant is stemmed or stemless, information that the implant is for a type of patient condition, information that the implant is for a particular bone, and press fit area, as a few non-limiting examples. The press fit area is the area where the implant will have its primary stability in the bone, waiting for bone ingrowth in this area. The strength of the press fit depends on the size/volume of the implant. The machine-learned model receives these examples of input data and groups, classifies, or generally performs the operation of a machine learning algorithm using the model parameters to determine information indicative of dimensions of the implant based on the implant characteristics.

As one example, the machine-learned model, using the model parameters, may determine a classification based on the input data. The classification may be associated with particular dimensions of the implant. For instance, the model parameters may include a plurality of clusters for each of a plurality of implants, where each cluster represents a multi-dimensional range of values for the input data. Each of the clusters may be associated with dimensions for respective implants. The machine-learned model may be configured to determine a cluster based on the input data and then determine the dimensions of the implant based on the determined cluster.

As described herein, FIGS. 9 through 13 are conceptual diagrams illustrating aspects of example machine-learning models. For ease of understanding, the example techniques are described with respect to FIG. 9 through 13. As one example, machine-learned model 902 of FIG. 9 is an example of a machine-learned model configured to perform example techniques described in this disclosure. As described in this disclosure, machine-learned model 720 of FIG. 7 is an example of machine-learned model 902. Any one or a combination of computing device 1002 (FIG. 10), server system 1104 (FIG. 11), and client computing device 1202 (FIG. 12) may be examples of a computing system configured to execute machine-learned model 902. In one or more examples, machine-learned model 902 may be generated with model trainer 1208 (FIG. 12) using example techniques described with respect to FIG. 13.

For instance, machine-learned model 902 may be configured to determine and output information indicative of information indicative of dimensions of an implant to be manufactured based on implant characteristics. A manufacturer may receive the information indicative of the dimensions of the implant and manufacture the implant based on the information indicative of the dimensions of the implant.

A computing system, applying machine-learned model 902, may be configured to obtain implant characteristics of an implant to be manufactured. The implant characteristics may include one or more of information that the implant is for a type of surgery, information that the implant is stemmed or stemless, information that the implant is for a type of patient condition, information that the implant is for a particular bone, and information of a press fit area of the implant. For example, the implant characteristics may include information indicating whether the implant will be used for an anatomical or reversed implant procedure, whether the implant will be stemmed or stemless, and the like. The implant characteristics may also include information indicating information such as the type of patient condition for which the implant will be used (e.g., fracture, osteoporosis, etc.), and/or information indicating the type of bone where the implant will be used (e.g., humerus), as some additional examples.

As explained above, the implant characteristics may be for an implant that is to be manufactured. The implant may be manufactured for keeping in stock at the manufacturer or hospital such that when that implant is needed, the implant is available. For instance, the implant may be for the humerus and stemmed, and the implant may be available in stock when needed. In some examples, the implant may be manufactured after the implant is needed (e.g., because the implant is not in stock). The implant to be manufactured need not be manufactured for a particular patient (e.g., the implant is not patient specific). However, in some examples, the implant may be a patient specific implant. Furthermore, the implants may be designed in pairs (e.g., glenoid and humeral implant) to cooperate with one another.

Moreover, the implant may not be patient specific, but may be meant for a particular group of people. The grouping of the people for which the implant is designed may be based on various factors such as race, height, gender, weight, etc. As an example, the implant characteristics may, in addition to or instead of including information that the implant is for a type of surgery, information that the implant is stemmed or stemless, information that the implant is for a type of patient condition, information that the implant is for a particular bone, and information of a press fit area of the implant, include information about a characteristic of a group of people such as race, weight, height, gender, etc.

The computing system, applying machine-learned model 902, may be configured to determine information indicative of dimensions of the implant based on the implant characteristics. For example, machine-learned model 902 may determine information indicative of a size and shape of the implant. As one example, machine-learned model 902 may determine information such as thickness, height, material, etc. of each of the components of the implant (e.g., length of stem, thickness of stem along the length, the material of the stem, shape, angles, etc.). In some examples, machine-learned model 902 may determine, in addition to or instead of the example information described above, information such as type of coating (e.g., hydroxyapatite (HAP) coating, porous titanium coating, etc.), type of finishing (e.g., blasted, mirror polished, coated, etc.), whether there is a graft window or not, location of the holes for suture and its location, whether locking screws are used or not (and if used type of locking screws), information indicative of thickness of the metal back on a glenoid implant, and information indicative of type of fixation (e.g., cemented, pressfit, locking screws, etc.).

The computing system, applying machine-learned model 902, may be configured to output the information indicative of the dimensions of the implant. A manufacturer may utilize the information indicative of the dimensions of the implant to manufacture the implant for use in surgery.

As an example, the computing system may be virtual planning system 701 of FIG. 7, and one or more storage devices 714 of virtual planning system 701 stores one or more machine-learned models 720 (e.g., object code of machine-learned models 720 that is executed by one or more processors 702 of virtual planning system 701). As described in this disclosure, one example of machine-learned models 720 is machine-learned model 902. One or more storage devices 714 stores implant design module 719 (e.g., object code of implant design module 719 that is executed by one or more processors 702).

A manufacturer may cause one or more processors 702 to execute implant design module 719 using one or more input devices 710. The manufacturer may enter, using one or more input devices 710, the implant characteristics of the implant to be manufactured. This is one example way in which the computing system (e.g., virtual planning system 701) may obtain the implant characteristics.

Executing implant design module 719 may cause one or more processors 702 to perform operations defined by one or more machine-learned models 720, including operations defined by machine-learned module 902. For example, one or more processors 702 may determine information indicative of dimensions of the implant to be manufactured based on the implant characteristics (e.g., size and shape).

One or more output devices 712 of virtual planning system 701 may be configured to output information indicative of the dimensions of the implant to be manufactured. For example, in some examples, one or more display devices 708 may be part of output devices 712, and one or more display devices 708 may be configured to present information indicative of the dimensions of the implant. In some examples, one or more output devices 712 may include one or more communication devices 706. One or more communication devices 706 may output the information indicative of the dimensions of the implant to one or more visualization devices, such as visualization device 213. In such examples, visualization device 213 may be configured to display the information indicative of the dimensions of the implant to be manufactured.

In some examples, one or more processors 702 may be configured to execute an application programming interface (API) for utilizing a computer-aided design (CAD) software. For example, the one or more processors 702 may utilize the API to provide the dimensions of the implant to be manufactured to the CAD software. The CAD software may generate a 3D model of the implant based on the dimensions of the implant. One or more processors 702 may utilize the CAD 3D model to generate an implant manufacturing file in a file format that an implant manufacturing machine can import and parse. The implant manufacturing machine may receive the implant manufacturing file and manufacture the implant based on the information in the implant manufacturing file.

The above example with respect to virtual planning system 701 is provided as one example and should not be considered limiting. For instance, other examples of a computing system such as computing device 1002 (FIG. 10) and client computing device 1202 (FIG. 12) may be configured to operate in a substantially similar manner.

In some examples, server system 1104 of FIG. 11 may be an example of a computing device. In such examples, server system 1104 may obtain the implant characteristics based on information provided by a manufacturer using client computing device 1102 of FIG. 11. Processing devices of server system 1104 may perform the operations defined by machine-learned model 902 (which is an example of machine-learned models 720 of FIG. 7). Server system 1104 may output the information indicative of the dimensions the implant back to client computing device 1102. Client computing device 1102 may then display information indicative of the dimensions of the implant or may further generate the implant manufacturing file.

In some examples, server system 1104 may generate the implant manufacturing file and transmit that implant manufacturing file to client computing device 1102 or directly to the implant manufacturing machine, bypassing client computing device 1102. However, even in such examples, server system 1104 may output information indicative of the dimensions of the implant such as output information indicative of the dimensions of the implant to the CAD software, where the CAD software may be executing on server system 1104 or elsewhere.

In some examples, machine-learned model 902 of the computing system may receive the implant characteristics and apply model parameters of the machine-learned model to the implant characteristics, as described in this disclosure with respect to FIG. 9. Machine-learned model 902 may determine the information indicative of the dimensions of the implant based on the application of the model parameters (e.g., based on applying the model parameters) of the machine-learned model.

There may be various ways in which machine-learned model 902 may apply the model parameters to determine the dimensions of the implant. As one example, machine-learned model 902, using the model parameters, may determine a classification based on the implant characteristics. The classification may be associated with a particular value for the dimensions of various components of the implant.

For example, the most appropriate pressfit area in case of fracture may be based on determining by comparison of osteolysis rate for several type of implant with distally or proximally pressfit considerations. In this example, the pressfit area may be a way in which machine-learned model 902 may classify the implants, and the classification may be based on the comparison of osteolysis rate.

In one or more examples, machine-learned model 902 may be trained using model trainer 1208 (FIG. 12), such as by using the techniques described with respect to FIG. 13, as one example. For example, model trainer 1208 may be configured to train machine-learned model 902 based on a machine learning dataset. The machine learning dataset may be information indicative of clinical outcomes for different types of implants and dimensions of available implants. For example, the information indicative of clinical outcomes may be information available from articles and publications of clinical outcomes, including information collected directly from physicians performing procedures. For each of the clinical outcomes, the machine learning dataset may include information of which implant was used, characteristics of that implant, for which procedure the implant was used, and characteristics of the patient on which the implant was used.

Examples of clinical outcomes include information indicative of survival rate, range of motion, pain level, etc. As one example, the information indicative of clinical outcomes may be information such as survival rate of the implant (e.g., how long the implant served its function before needing to be replaced). Model trainer 1208 may utilize the survival rate of various implants used for a particular type of fracture. The size and shapes of the implants may impact the survival rate, and model trainer 1208 may be configured to train machine-learned model 902 to determine size and shapes of the implants that increase the survival rate.

The combination of these criteria (e.g., which implant, characteristics of implant, procedure, and characteristics of the patient) may all influence the outcome. For example, a younger, healthier patient who received an implant for a fracture may have a different outcome than an older, unhealthy patient who received the same implant for the same type of fracture. Accordingly, model trainer 1208 may be configured to account for all these different criteria in generating the model parameters.

For example, training data 1302 may include information such as patient ages, weight, height, smoker or not, types of diseases, etc., types of implants, and the like. Example input data 1304 may be the actual information about the patient ages, weight, height, smoker or not, types of diseases, and the implants used in patients. Output data 1308 may be the clinical outcomes for the patients that make up the example input data 1304.

In some examples, the clinical outcomes for the patients may be a multi-factor comparison. For instance, length of surgery, survival rate, type of fracture, etc. may all be factors of output data 1308. As one example, output data 1308 may indicate that for a particular type of surgery and a particular type of fracture, that the result was implanting a particular implant. For a different type of surgery, a different type of fracture, and a different implant, the result may be different, and represented in output data 1308.

Objective function 1310 may utilize the example input data 1304 and the output data 1308 to determine model parameters for machine-learned model 902. For example, the model parameters may be weights and biases, or other example parameters described with respect to FIGS. 9-13. The result of the training may be that machine-learned model 902 is configured with model parameters that can be used to determine dimensions of an implant to be manufactured.

In this way, this disclosure describes example techniques utilizing computational processes for determining dimensions of an implant for manufacturing. The example techniques described in this disclosure are based on machine learning datasets that may be extremely vast with more information than could be accessed or processed by a human designer or manufacturer without access to the computing system that uses machine-learned model 902. For instance, human designers or manufacturers may not be able to determine that some implant dimensions have already been tried and have not worked for various reasons. Manufactures or designers may end up designing and manufacturing implants that were otherwise known to be defective, or at least less effective than others. With the example techniques described in this disclosure, machine-learned model 902 may determine information indicative of dimensions of the implant (e.g., diameter of the metaphysis, angle of the stem, shape of the glenoid, length of the glenoid plug, etc.) to be manufactured based on the implant characteristics and avoid bad implant concepts.

Even if a person were to access and review the information from the dataset, the person may still not be able to, given the vast amount of information, construct a technique that accurately accounts for all the different patient information and implant characteristics. However, machine-learned model 902 may be configured to utilize the vast amount of information as a way to determine dimensions of an implant. Moreover, using machine-learned model 902 allows for a scalable, extensible computing system that can be updated with new information periodically to create a better version of machine-learned model 902. A person may not have the ability to update his/her understanding of what the dimensions should be, much less update as quickly as machine-learned model 902 can be updated.

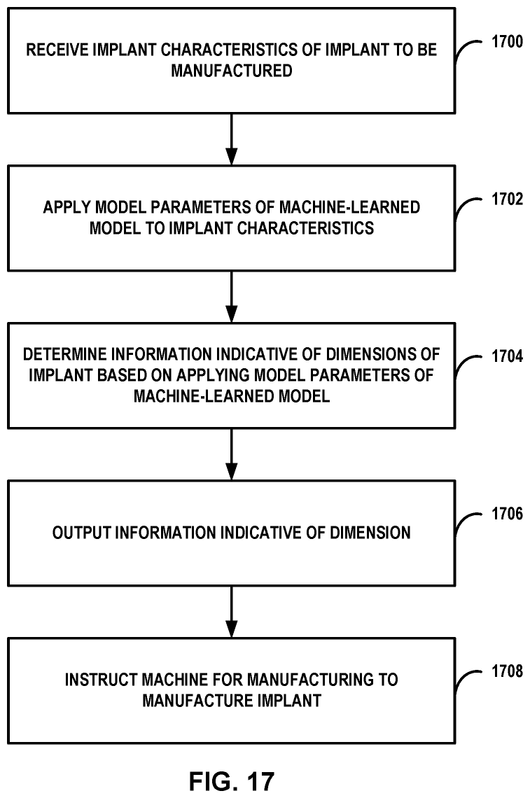

FIG. 17 is a flowchart illustrating an example method of determining information indicative of dimensions of an implant. For ease of description, the example of FIG. 17 is described with respect to FIG. 7 and machine-learned model 902 of FIG. 9, which is an example of machine-learned model 720 of FIG. 7. However, the example techniques are not so limited.

As illustrated in FIG. 7, storage device(s) 714 stores machine-learned model(s) 720, an example of which is machine-learned model 902. One or more processors 702 may access and execute machine-learned model 902 to perform the example techniques described in this disclosure. One or more storage devices 714 and one or more processors 702 may be part of the same device or may be distributed across multiple devices. For instance, virtual planning system 701 is an example of a computing system configured to perform the example techniques described in this disclosure.

One or more processors 702 (e.g., using machine-learned model 902) may receive implant characteristics of an implant (1700). For example, implant characteristics of the implant may include one or more of information that the implant is for a type of surgery, information that the implant is stemmed or stemless, information that the implant is for a type of patient condition, information that the implant is for a particular bone, and/or information of a press fit area of the implant. The press fit area is the area where the implant will have its primary stability in the bone, waiting for bone ingrowth in this area. The strength of the press fit depends on the size/volume of the implant. The following are some additional examples of implant characteristics: length of the stem in case of a revision stem, graft window for revision or fracture cases, locking screw to lock the stem in the humerus in case of revision, convertible prosthesis, monolithic or modular prosthesis, stem shape that mimics the internal shape of the humerus or not, etc. A manufacturer may provide information of the implant characteristics using input devices 710 as an example.

One or more processors 702 may apply model parameters of machine-learned model 902 to the implant characteristics (1702). In some examples, the model parameters of machine-learned model 902 are generated based on a machine learning dataset. For example, the machine learning dataset includes one or more of information indicative of clinical outcomes for different types of implants and dimensions of available implants. The information indicative of clinical outcomes may be information available from articles and publications of clinical outcomes, examples of which include information collected directly from physicians performing procedures. Examples of information available from articles includes survival rate, range of motion, pain level, revision rate, dislocation rate, and infection rate. The above examples of information available from articles may be for each of a plurality of patient characteristics like mean age, gender, diseases, dominant arm, etc.

As an example of ease with understanding, a manufacturer may want to design an implant with a particular length for the stem for men and for fracture. In this example, machine-learned model 902 may have been trained with information from publications about the outcomes of different implants within men. The result of the training may be model parameters that one or more processors 702, via machine-learned model 902, are to apply to implant characteristic information such as length of stem, for fracture, and for men. In this example, machine-learned model 902 may scale, modify, weight, etc. the input information based on the model parameters.

One or more processors 702 may be configured to determine information indicative of dimensions of the implant based on applying model parameters of machine-learned model 902 (1704). For example, the result of the applying the model parameters may be information indicative of external size and shape of the implant. In some examples, machine-learned model 902 may determine, in addition to or instead dimensions of the implant, information such as type of coating (e.g., hydroxyapatite (HAP) coating, porous titanium coating, etc.), type of finishing (e.g., blasted, mirror polished, coated, etc.), whether there is a graft window or not, location of the holes for suture and its location, whether locking screws are used or not (and if used type of locking screws), information indicative of thickness of the metal back on a glenoid implant, and information indicative of type of fixation (e.g., cemented, pressfit, locking screws, etc.).

One or more output devices 712 may be configured to output the information indicative of the dimensions of the implant to be manufactured (1706). For example, one or more processors 702 may output the information indicative of the external size and shape of the implant to one or more output devices 712. One or more output devices 712 may display the dimensions of the implant (e.g., in examples where display device 708 is part of output devices 712). In some examples, one or more output devices 712 may output information indicative of the dimensions of the implant to another device such as visualization device 213 for display.

In some examples, one or more processors 702 may generate a 3D model of the implant (e.g., such as using CAD software). Display device 708 or visualization device 213 may display the 3D model of the implant, and a surgeon or other health professional may confirm that the 3D model of the implant should be manufactured.

One or more processors 702 may instruct a machine for manufacturing to manufacture the implant (1708). For example, one or more processors 702 may cause output devices 712 to output the CAD 3D model of the implant to generate an implant manufacturing file in a file format that an implant manufacturing machine can import and parse. The implant manufacturing machine may receive the implant manufacturing file and manufacture the implant based on the information in the implant manufacturing file.

During the preoperative phase of the surgical procedure, a surgeon may use surgery planning module 718 to develop a surgical plan for the surgical procedure. As discussed elsewhere in this disclosure, processing device(s) 1004 (FIG. 10) may execute instructions that cause computing device 1002 (FIG. 10) to provide the functionality ascribed in this disclosure to surgery planning module 718. The surgical plan for the surgical procedure may specify a series of steps to perform during the surgical procedure, as well as sets of surgical items to use during various ones of the steps of the surgical procedure. As the surgeon develops the surgical plan using surgery planning module 718, surgery planning module 718 may allow the surgeon to select among various surgical options for each step of the surgical procedure.

Example types of surgical options for a step of the surgical procedure may include a range of surgical items, such as orthopedic prostheses (i.e., orthopedic implants), that the surgeon may use during the step of the orthopedic surgery. For instance, there may be a range of glenoid prostheses from which the surgeon may choose a glenoid prothesis. Other types of example surgical options include attachment positions for a specific orthopedic prosthesis. For instance, in an example involving a glenoid prosthesis, virtual planning system 701 may allow the surgeon to select an attachment position for the glenoid prosthesis from a range of attachment positions that are more medial or less medial, more anterior or less anterior, and so on.

Selecting the correct surgical options for a surgical procedure may be vital to the success of the surgical procedure. For example, selecting an incorrectly sized orthopedic prosthesis may lead to the patient experiencing pain or limited range of motion. In another example, selecting an incorrect attachment point for an orthopedic prosthesis may lead to loosening of the orthopedic prosthesis over time, which may eventually require a revision surgery.

Different patients have different anatomic parameters and different patient characteristics. The anatomic parameters for the patient may be descriptive of the patient's anatomy at the surgical site for the surgical procedure. The patient characteristics may include one or more characteristics of the patient separate from the anatomic parameter data for the patient. Because patients have different anatomic parameters and different patient characteristics, surgeons may need to select different surgical options for different patients.

Because there may be a very large number of surgical options from which a surgeon can choose, it may be challenging for the surgeon to select a combination of surgical options that is best for a specific patient. Accordingly, it may be desirable for a surgical planning system, such as surgery planning module 718, to suggest appropriate surgical options for the patient, given the anatomic parameters and patient characteristics of the patient.

However, implementing a computerized system for suggesting appropriate surgical options presents significant challenges. For instance, the number of combinations of selectable surgical options may grow exponentially, which may result in a significant draw on the memory and computational resources of any computing system implementing such a system. Moreover, there is typically a range of acceptable surgical options for any given patient. In other words, there might not be one right answer to the question of which set of surgical options should be used in a surgical procedure. Thus, even if the implementation problems associated with the potentially large number of combinations can be addressed, there may be a problem of how to account for the ranges of acceptable surgical options. Computerized solutions for suggesting appropriate surgical options during an intraoperative phase of a surgical procedure may present even more challenges, such as how to account for surgical options that can no longer be unselected or how to suggest surgical options when a surgical plan changes during the surgical procedure.

This disclosure describes techniques that may address one or more of these problems. As described herein, surgery planning module 718 (FIG. 7) may generate data specifying a surgical plan for a surgical procedure. The surgical plan may specify a series of steps that are to be performed during the surgical procedure. Furthermore, for one or more of the steps of the surgical procedure, the surgical plan may specify one or more surgical parameters. A surgical parameter of a step of the surgical procedure may be associated with a range of surgical options from which the surgeon can choose. For example, a surgical parameter of a step of implanting a glenoid prosthesis may be associated with a range of differently sized glenoid protheses.

Surgery planning module 718 may use one or more machine-learned models 720 to determine sets of recommended surgical options for one or more surgical parameters of one or more steps of a surgical procedure. For instance, surgery planning module 718 may use a different one of machine-learned models 720 to determine different sets of recommended surgical options for different surgical parameters. In some instances, a set of recommended surgical options includes a plurality of recommended surgical options. As the surgeon plans the surgical procedure, surgery planning module 718 may receive indications of the surgeon's selection of surgical options for the surgical parameters of the steps of the surgical procedure. Surgery planning module 718 may determine whether a selected surgical option is among the recommended surgical options for a surgical parameter. If the selected surgical option is not among the recommended surgical options for the surgical parameter, surgery planning module 718 may output a warning indicating that the selected surgical option is not among the recommended surgical options.

Thus, by determining a set of recommended surgical options for a surgical parameter and warning the surgeon when the selected surgical option is not among the set of recommended surgical options, the problem of how to implement a computerized system to determine which of the surgical options is the single best surgical option may be avoided. Because a machine-learned model is expected to determine a set of one or more recommended surgical options, as opposed to the single best surgical option, less training data may be required in order to train the machine-learned model to a workable state.

Furthermore, as described herein, the user's selection of a surgical option for a first surgical parameter may serve as input to a machine-learned model that generates a set of recommended surgical options for a second surgical parameter. Thus, the machine-learned model may generate the set of recommended surgical options for the second surgical parameter given the surgical option selected for the first surgical parameter. For example, a surgeon may select a specific glenoid implant as a first surgical parameter. In this example, data indicating the specific glenoid implant may serve as input to a machine-learned model that generates a set of recommended surgical options for a surgical parameter corresponding to a humeral implant.

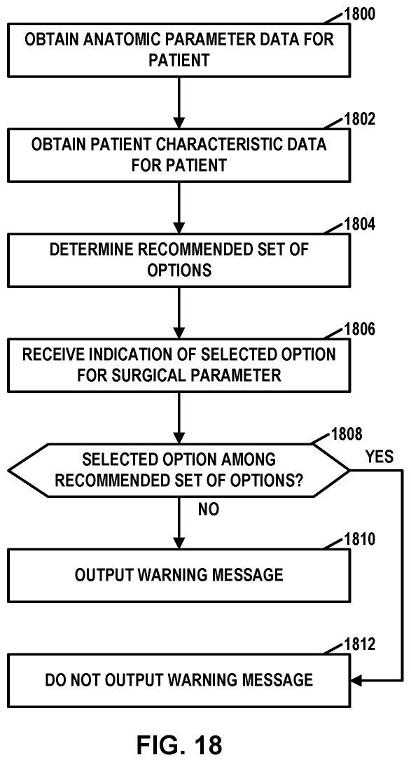

FIG. 18 is a flowchart illustrating an example operation in accordance with one or more techniques of this disclosure. FIG. 18 is presented as an example. Other examples in accordance with the techniques of this disclosure may include more, fewer, or different actions, or the actions may be performed in different orders. Surgery planning module 718 may perform the operation of FIG. 18 using different machine-learned models 720 for different surgical parameters. In other words, surgery planning module 718 may perform the operation of FIG. 18 multiple times for different steps of the surgical procedure and/or different surgical parameters.

In the example of FIG. 18, surgery planning module 718 may obtain anatomic parameter data for the patient (1800). The anatomic parameter data for the patient may include data that is descriptive of the patient's anatomy at the surgical site for the surgical procedure. Because different surgical procedures involve different surgical sites (e.g., a shoulder in a shoulder arthroplasty and an ankle in an ankle arthroplasty), the anatomic parameter data may include different data for different types of surgical procedures. In the context of shoulder arthroplasty surgeries, the anatomic parameter data may include a wide variety of data that is descriptive of the patient's anatomy at the surgical site for the surgical procedure. For instance, the anatomic parameter data may include data regarding one or more of a status of a bone of a joint of the patient that is subject to the surgical procedure; a status of muscles and connective tissue of the joint of the patient, and so on. In some examples involving the shoulder joint, other example types of anatomic parameter data may include one or more of the following:
A distance from a humeral head center to a glenoid center.
A distance from the acromion to the humeral head.
A scapula critical shoulder sagittal angle (i.e., an angle between the lines mentioned above for the CSA, as the lines would be seen from a sagittal plane of the patient).
A glenoid coracoid process angle (i.e., an angle between (1) a line from a tip of the coracoid process to a most inferior point on the border of the glenoid cavity of the scapula, and (2) a line from the most inferior point on the border of the glenoid cavity of the scapula to a most superior point on the border of the glenoid cavity of the scapula).
An infraglenoid tubrical angle (i.e., an angle between (1) a line extending from a most inferior point on the border of the glenoid cavity to a greater tuberosity of the humerus, and (2) a line extending from a most superior point on the border of the glenoid cavity to the most inferior point on the border of the glenoid cavity).
A scapula acromion index.
A humerus orientation (i.e., a value indicating an angle between (1) a line orthogonal to the center of the glenoid, and (2) a line orthogonal to the center of the humeral head, as viewed from directly superior to the patient).
A humerus direction.
A measure of humerus subluxation.
A humeral head best fit sphere (i.e., a measure (e.g., a root mean square) of conformance of the humeral head to a sphere).

Furthermore, in the example of FIG. 18, surgery planning module 718 may obtain patient characteristic data for the patient (1802). The patient characteristic data may include data regarding one or more characteristics of the patient separate from the anatomic parameter data for the patient. In other words, the patient characteristic data may include data regarding the patient that is not descriptive of the patient's anatomy at the surgical site for the surgical procedure. Example types of patient characteristic data may include one or more of the following: an age of the patient, a disease state of the patient, a smoking status of the patient, a state of an immune system of the patient, a diabetes state of the patient, desired activities of the patient, and so on. The state of the immune system of the patient may indicate whether or not the patient is in a state of immunodepression.

Surgery planning module 718 may use a machine-learned model (e.g., one of machine-learned models 720) to determine a set of recommended surgical options for a surgical parameter (1804). In some examples, the set of recommended surgical options may correspond to options that other surgeons are likely to use when planning the surgical procedure on the patient, given the patient characteristics data for the patient and/or the anatomic parameter data for the patient. Surgery planning module 718 may provide the anatomic parameter data and/or the patient characteristic data as input to the machine-learned model. In some examples, surgery planning module 718 may also provide different sets of anatomic parameter data and/or patient characteristic data to machine-learned models for different surgical parameters. Furthermore, in some examples, surgery planning module 718 may provide data indicating one or more previously selected surgical options as input to the machine-learned model.

The machine-learned model may be implemented in one of a variety of ways. For instance, the machine-learned model may be implemented using one or more of the types of machine-learned models described elsewhere in this disclosure, such as with respect to FIG. 9. For instance, in one example, the machine-learned model may include a neural network. In this example, different input neurons in a set of input neurons (e.g., some or all of the input neurons of the artificial neural network) of the neural network may receive different types of input data (e.g., anatomic parameter data, patient characteristic data, data indicating previously selected surgical options, etc.). Furthermore, in this example, the neural network has a set of output neurons (e.g., some or all of the output neurons of the artificial neural network) corresponding to different surgical options in a plurality of surgical options. Each of the output neurons in the set of output neurons may be configured to output a confidence value indicating a level of confidence that a set of reference surgeons would have selected the surgical option corresponding to the output neuron.

Virtual planning system 701 may identify the recommended surgical options based on the confidence values output by the output neurons. For instance, in some examples, virtual planning system 701 may determine that the recommended surgical options are surgical options whose corresponding output neurons generated confidence values that are above a particular threshold. In other examples, virtual planning system 701 may rank the surgical options based on the confidence values generated by the output neurons corresponding to the surgical options and select a given number of the highest-ranked surgical options as the set of recommended options.

As noted above, each of the output neurons may be configured to output a confidence value indicating a level of confidence that a set of reference surgeons would have selected the surgical option corresponding to the output neuron. To ensure that the output neurons output confidence values indicating levels of confidence that the set of reference surgeons would have selected the surgical options corresponding to the output neurons, the neural network may have been trained using training data that indicate surgical options selected by the reference surgeons when given various sets of patient characteristic data and/or anatomic parameter data for the patient.

The reference surgeons may be determined in any of one or more ways. For example, the reference surgeons may be a set of surgeons recognized as experts in performing the orthopedic surgery that the user is planning. In some examples, the reference surgeons may be a set of surgeons who are working within the same insurance network, same hospital, or same region.

In some examples where the machine-learned model includes a neural network, surgery planning module 718 may train the neural network using training data that comprises training data pairs. Although described as being trained by surgery planning module 718, the neural network may, in some examples, be trained by another application and/or model trainer 1208 (FIG. 12). Each training data pair corresponds to a different performance of the surgical procedure by one of the reference surgeons. Each training data pair includes an input vector (e.g., example input data 1304 (FIG. 13) and a target value (e.g., labels 1306). The input vector of a training data pair may include values for each input neuron of the neural network. The target value of a training data pair may specify a surgical option that was actually used in the surgical step corresponding to the machine-learned model.

In some examples, surgery planning module 718 may use the training process 1300 (FIG. 13) to train the neural network. For instance, in some examples, to train the neural network using the training data, surgery planning module 718 may perform a forward pass through the neural network using the input vector of a training data pair. Surgery planning module 718 may then compare the confidence values generated by the output neurons to the target value to determine an error value, e.g., using objective function 1310 (FIG. 13). Surgery planning module 718 may then use a backpropagation algorithm to modify weights of neurons of the neural network based on the error value. Surgery planning module 718 may repeat this process for different training data pairs in the training data. In some examples, surgery planning module 718 may receive new training data pairs and may continue training the neural network as more surgical procedures are completed.

In some examples, surgery planning module 718 may automatically generate training data pairs. As noted elsewhere in this disclosure, surgery planning module 718 may be used to generate surgical plans and generating surgical plans may involve selecting surgical options. Surgery planning module 718 may take the anatomic parameter data, patient characteristic data, and selected surgical option for a specific surgical parameter of a specific surgical step of such a surgical plan generated by a reference surgeon and generate a training data pair based on this data. Because surgical plans generated using surgery planning module 718 may share the same surgical steps (and data structures identifying the surgical steps), surgery planning module 718 may apply data generated across instances of the same surgical step in different instances of the same surgical procedure. In other words, surgery planning module 718 may generate training data pairs based on anatomic parameter data, patient characteristic data, and selected surgical options for the specific step in difference instances of the same surgical procedure. Thus, surgery planning module 718 may use the training data pair to train the machine-learned model for the specific surgical parameter of the specific surgical step. In this way, as the reference surgeons plan more and more surgical procedures, surgery planning module 718 may generate more and more training data pairs that surgery planning module 718 may use to continue training machine-learned models.

In other examples, machine-learned model 720 may be implemented as one or more support vector machine (SVM) models, Bayesian network models, decision tree models, or random forests, other types of machine-learned model. In examples where machine-learned model 720 is implemented using SVM models, there may be a plurality of separate SVM models for different surgical options. In this example, the SVM model of a surgical option may classify the surgical option as being part of the recommended set of surgical options or not in the recommended set of surgical options. In examples where machine-learned model 720 includes a set of decision trees, the set of decision trees may include decision trees that generate output indicating whether or not a surgical option is or is not in the recommended set of surgical options.

In examples where machine-learned model 720 includes a Bayesian network, the Bayesian network may take the planning parameters as inputs and the training will be done by optimization on a validation database (a set of “regular” planning). Then, for testing if a selected surgical option is or is not in a recommended set of surgical options, the surgery planning module 718 may project the selected surgical option into a space represented by the possible surgical options, and then determine whether that projection is within the recommended set of surgical options.

Furthermore, in the example of FIG. 18, surgery planning module 718 may receive an indication of a selected surgical option for the surgical parameter (1806). For example, surgery planning module 718 may receive an indication of voice input, touch input, button-push input, etc., that specifies the selected surgical option.

Surgery planning module 718 may then determine whether the selected surgical option is among the set of recommended surgical options (1808). Based on determining that the selected surgical option is not among the set of recommended surgical options (“NO” branch of 1808), surgery planning module 718 may output a warning message to the user (1810). On the other hand, based on determining that the selected surgical option is among the set of recommended surgical options (“YES” branch of 1808), surgery planning module 718 may not output the warning message (1812).

Surgery planning module 718 may output the warning message in one or more ways. For instance, in one example, surgery planning module 718 may output the warning message as text or graphical data in an MR visualization. In another example, surgery planning module 718 may output the warning message as text or graphical data in a 2-dimensional display. The warning message may indicate to the user that the reference surgeons are unlikely to have chosen the selected option for the patient, given the patient characteristic data for the patient. In some examples, the warning message on its own is not intended to prevent the user from using the selected surgical option during the surgical procedure on the patient. Thus, in some examples, the warning message does not limit the choices of the user. Rather, the warning message may help the user understand that the selected surgical option might not be the surgical option that the reference surgeons would typically choose.

In some examples, surgery planning module 718 may perform the operation of FIG. 18 during an intraoperative phase of the surgical procedure. In such examples, surgery planning module 718 may receive an indication of a selection of a surgical option for a surgical parameter during the intraoperative phase of the surgical procedure. In some examples, this selected surgical option may be different from the surgical option selected for the same surgical parameter of a step of the surgical procedure during the preoperative phase of the surgical procedure. Accordingly, in such examples, surgery planning module 718 may output a warning if the intraoperatively selected surgical option is not among a recommended set of surgical options. In this way, the surgeon may still have some level of flexibility to select among surgical options during the surgical procedure (e.g., due to unavailability of surgical item or other reason).

In some examples, the surgical plan for the surgical procedure may change while the surgeon is performing the surgical procedure. For instance, the surgeon may need to change the surgical plan for the surgical procedure during the intraoperative phase of the surgical procedure upon discovering that the patient's anatomy is different than assumed during the preoperative phase of the surgical procedure. Accordingly, surgery planning module 718 may update the surgical plan for the surgical procedure during the intraoperative phase of the surgical procedure. In some examples, surgery planning module 718 may determine a modified surgical plan for the surgical procedure in accordance with one or more of the examples described in PCT application no. PCT/US2019/036993, filed Jun. 13, 2019, the entire content of which is incorporated by reference. The updated surgical plan for the surgical procedure may have different steps from the original surgical plan for the surgical procedure. In accordance with an example of this disclosure, surgery planning module 718 may perform the operation of FIG. 18 for surgical parameters of steps of the updated surgical plan for the surgical procedure. Thus, the surgeon may be able to receive information during the surgical procedure about whether selected surgical options are among sets of recommended surgical options for the patient.

As noted above, in some examples, one or more of the machine-learned models may receive indications of previously selected surgical options. Thus, a machine-learned model may use information about the previously selected surgical options when determining the set of recommended surgical options for a surgical parameter. Hence, in some examples, surgery planning module 718 may use a second machine-learned model to determine a second set of recommended surgical options for a second surgical parameter, wherein the anatomic parameter set for the patient, the patient characteristic data for the patient are input to the machine-learned model, and the selected surgical option for a first surgical parameter are input to the second machine-learned model. Thus, the set of recommended surgical options may be different depending on a previously selected surgical option. For instance, in one example, the set of recommended surgical options may include a plurality of humeral prostheses. In this example, the plurality of humeral prostheses may be different depending on which glenoid prosthesis was selected by the surgeon.

Because the machine-learned model may be designed to accept only those ones of the previously selected surgical options that are material to the determination of the recommended surgical options, it may be unnecessary to evaluate combinations of all surgical options at once. In this way, examples of this disclosure may avoid problems associated with large numbers of potential combinations of surgical options, which may be costly in terms of computing resources.

It is noted that providing data indicating previously selected surgical options as input to machine-learned models may create dependencies in the order in which the surgeon selects surgical options. However, in some examples, if surgery planning module 718 uses a machine-learned model to determine a set of recommended surgical options and the surgeon has not indicated a selection of a surgical option that the machine-learned model uses as input, the machine-learned model may be trained to generate the set of recommended surgical options such that the set of recommended surgical options includes no recommended surgical options. In other examples, surgery planning module 718 may output the warning message without using the machine-learned model when the surgeon selects the surgical option. In this way, the resulting warning message may make the surgeon aware that surgery planning module 718 cannot accurately provide guidance about whether the selected surgical option is among a set of recommended surgical options.

An estimated amount of operating room (OR) time for a surgical procedure to be performed on a patient may be or include an estimate of an amount of time that an OR will be in use during performance of the surgical procedure on the patient. Estimating the amount of OR time for a surgical procedure may be important for a variety of reasons. For example, because hospitals typically have a limited number of ORs, it may be important for hospitals to know the estimated amounts of OR time for surgical procedures in order to determine how best to schedule surgical procedures in the ORs. That is, hospital administrators may want to maximize utilization of ORs through appropriate scheduling of surgical procedures. Appropriate estimation of amounts of OR time for some types of orthopedic surgical procedures may be especially important given that orthopedic surgical procedures can be lengthy and are also frequently non-urgent. Because orthopedic surgical procedures are frequently non-urgent, there may be greater flexibility in scheduling orthopedic surgical procedures relative to other types of surgical procedures, such as oncology surgeries, organ transplant surgeries, and so on.

In addition to using estimates of amounts of OR time for purposes of optimizing OR utilization, an accurate estimate of an amount of OR time for a surgical procedure may be important in understanding the risk of the patient acquiring an infection during the surgical procedure. The risk of the patient acquiring an infection increases with increased amounts of OR time. The patient, the surgeon, and hospital administration need to understand the risk of infection before undertaking the surgical procedure.

Currently, surgeons use their professional judgment in estimating amounts of OR time for surgical procedures. However, some surgeons may be unable to accurately estimate amounts of OR times for surgical procedures. For instance, some surgeons may estimate amounts of OR time for surgical procedures that are too long or too short, which may result in sub-optimal OR utilization. It may be especially difficult to estimate amounts of OR times for certain types of orthopedic surgeries, such as shoulder arthroplasties and ankle arthroplasties, because of the high number of surgical options available to surgeons. For instance, in one example involving a shoulder arthroplasty, a surgeon may choose between a stemmed or stemless humeral implant. In this example, it may take different amounts of time to implant a stemmed humeral implant versus a stemless humeral implant. In another example involving a shoulder arthroplasty, a surgeon may choose between different types of glenoid implants. In this example, different types of glenoid implants may require different amounts of reaming, different types of bone grafts, and so on. Furthermore, in another example involving a shoulder arthroplasty, arthritic shoulders commonly develop osteophytes that should be accounted for during the shoulder arthroplasty. Thus, because of the large number of surgical options available to a surgeon, it may be difficult for the surgeon to accurately estimate the amount of OR time for a surgical procedure.

In addition to the variety of surgical options available to a surgeon, it may be difficult to estimate an amount of OR time for a surgical procedure to be performed in a specific patient because of various patient-specific parameters. For instance, it may take different amounts of time to perform the same surgical procedure on diabetic patients as opposed to non-diabetic patients.

Computerized techniques for scheduling ORs have previously been developed. In some instances, computerized techniques for scheduling ORs simply accept a surgeon's estimate of the amount of OR time for a surgical procedure. In some instances, computerized techniques for scheduling ORs use default, static estimates of amounts of OR time for surgical procedures. However, because of the high degree of variability within even the same type of surgical procedure, the estimated amounts of time used in such computerized techniques may be quite inaccurate, leading to poor utilization of ORs. Moreover, such techniques do not provide for a way to update the estimated amount of OR time during an intraoperative phase of the surgical procedure.

Techniques of this disclosure may address one or more of these issues. In accordance with one or more techniques of this disclosure, surgery planning module 718 (FIG. 7) may use one or more machine-learned models 720 (FIG. 7) to estimate an amount of OR time for a surgical procedure. Surgery planning module 718 may estimate the amount of OR time for the surgical procedure during a preoperative phase (e.g., preoperative phase 302 (FIG. 3)) of the surgical procedure. Furthermore, in some examples where surgery planning module 718 estimates the amount of OR time for the surgical procedure during the preoperative phase, virtual planning system 701 or other computing systems may determine an operating room schedule based on the estimated amount of OR time for the surgical procedure. In some examples, surgery planning module 718 may estimate an updated amount of OR time during an intraoperative phase (e.g., intraoperative phase 306 (FIG. 3)) of the surgical procedure.

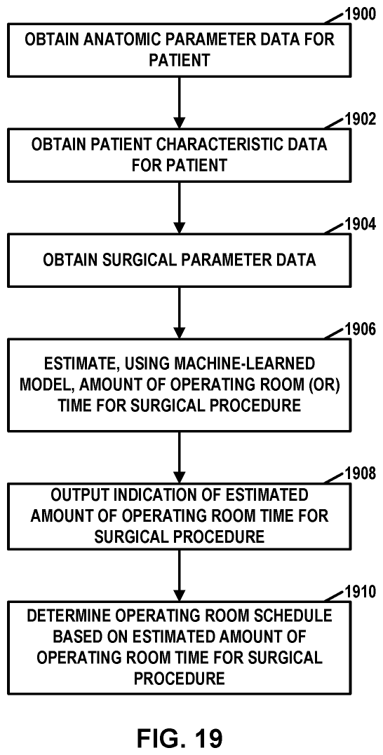

FIG. 19 is a flowchart illustrating an example operation of virtual planning system 701 to determine an estimated OR time for a surgical procedure to be performed on a patient, in accordance with one or more techniques of this disclosure. The estimated OR time for a surgical procedure to be performed on a patient is an estimate of an amount of time that an OR will be in use during performance of the surgical procedure on the patient. The operation of FIG. 19 is presented as an example. Other examples of this disclosure may include more, fewer, or different actions, or actions that are performed in different orders. For instance, in some examples, virtual planning system 701 does not perform one or more of actions 1908 and 1910.

As shown in the example of FIG. 19, surgery planning module 718 may obtain anatomic parameter data for the patient (1900). The anatomic parameter data for the patient may include data that is descriptive of the patient's anatomy at the surgical site for the surgical procedure. Because different surgical procedures involve different surgical sites (e.g., a shoulder in a shoulder arthroplasty and an ankle in an ankle arthroplasty), the anatomic parameter data may include different data for different types of surgical procedures. In the context of shoulder arthroplasty surgeries, the anatomic parameter data may include any of the types of anatomic parameter data described elsewhere in this disclosure. For instance, the anatomic parameter data may include data regarding one or more of a status of a bone of a joint of the current patient that is subject to the surgical procedure; and a status of muscles and connective tissue of the joint of the current patient, and so on.

Furthermore, in the example of FIG. 19, surgery planning module 718 may obtain patient characteristic data for the patient (1902). The patient characteristic data may include data regarding one or more characteristics of the patient separate from the anatomic parameter data for the patient. In other words, the patient characteristic data may include data regarding the patient that is not descriptive of the patient's anatomy at the surgical site for the surgical procedure. Example types of patient characteristic data may include one or more of the following: an age of the patient, a disease state of the patient, a smoking status of the patient, a state of an immune system of the patient, a diabetes state of the patient, desired activities of the patient, and so on. The state of the immune system of the patient may indicate whether or not the patient is in a state of immunodepression.

Surgery planning module 718 may also obtain surgical parameter data for the surgical procedure (1904). The surgical parameter data may include data regarding a type of surgical procedure, as well as data indicating selected surgical options for the surgical procedure. For instance, the surgical parameter data may include data indicating any of the types of surgical options described elsewhere in this disclosure. For instance, example types of surgical parameter data may include one or more of parameters of a surgeon selected to perform the surgical procedure, a type of the surgical procedure, a type of an implant to be implanted during the surgical procedure, a size of the implant, an amount of bone to be removed during the surgical procedure, and so on.

Surgery planning module 718 may estimate, using one or more of machine-learned models 720, an amount of OR time for the surgical procedure based on the patient characteristic data, the anatomic parameter data, and the surgical parameter data (1906). Surgery planning module 718 may estimate the amount of OR time in one or more of various ways.

The one or more machine-learned models 720 may be implemented in accordance with one or more of the example types of machine-learned models described with respect to FIG. 9, and elsewhere in this disclosure. For instance, in one example, surgery planning module 718 may estimate the amount of OR time for the surgical procedure using a single artificial neural network. For ease of explanation, this disclosure may refer to artificial neural networks simply as neural networks and may refer to artificial neurons simply as neurons. In this example, the neural network may include an input layer, an output layer, and one or more hidden layers between the input layer and the output layer. Each layer of the neural network includes a separate set of neurons. Neurons in the input layer are known as input neurons and neurons in the output layer are known as output neurons. In an example where surgery planning module 718 estimates the amount of OR time for the surgical procedure using a single neural network, different input neurons in the input layer of the neural network may receive, as input, different data in the anatomic parameter data, patient characteristic data, and surgical parameter data.

In some examples, the input layer may include input neurons that receive input data separate from and additional to data in the anatomic parameter data, the patient characteristic data, and the surgical parameter data. For example, an input neuron may receive input data indicating an experience level of the surgeon performing the surgical procedure. In another example, an input neuron may receive data indicating a region in which the surgeon practices.

The output neurons of the neural network may output various types of data. For instance, in some examples, the output neurons of the neural network include an output neuron that outputs an indication of the estimated amount of OR time for the surgical procedure. In such examples, surgery planning module 718 may train the neural network using training data that comprises training data pairs. Each training data pair corresponds to a different performance of the surgical procedure. Each training data pair includes an input vector (e.g., example input data 1304 (FIG. 13)) and a target value (e.g., labels 1306 (FIG. 13). The input vector of a training data pair may include values for each input neuron of the neural network. The target value of a training data pair may specify an amount of time that was actually required to perform the surgical procedure corresponding to the training data pair. Although this example and other examples of this disclosure are described with respect to surgery planning module 718 training this neural network or other neural networks, model trainer 1208 or another application may train such neural networks.

To train the neural network using the training data, surgery planning module 718 may perform a forward pass through the neural network using the input vector of a training data pair. Surgery planning module 718 may then compare the indication of the amount of OR time for the surgical procedure generated by the output neuron to the target value to determine an error value. Surgery planning module 718 may then use a backpropagation algorithm to modify weights of neurons of the neural network based on the error value. Surgery planning module 718 may repeat this process for different training data pairs in the training data. In some examples, surgery planning module 718 may receive new training data pairs and may continue training the neural network as more surgical procedures are completed.

In another example, the output neurons of the neural network correspond to different time periods. For instance, in this example, a first output neuron may correspond to an OR time of 0-29 minutes, a second output neuron may correspond to an OR time of 30-59 minutes, a third output neuron may correspond to an OR time of 60-89 minutes, and so on. In other examples, the output neurons may correspond to time periods of greater or less duration. In some examples, the time periods corresponding to the output neurons all have the same duration. In some examples, two or more of the time periods corresponding to the output neurons of the same neural network may be different.

In examples where the output neurons of the neural network include output neurons that correspond to different time periods, the output neurons may generate confidence values. A confidence value generated by an output neuron may be indicative of a level of confidence that the surgical procedure will end within the time period corresponding to the output neuron. For example, the confidence value generated by the output neuron corresponding to the OR time of 30-59 minutes indicates a level of confidence that the surgical procedure will end at some time between 30 and 59 minutes after the surgical procedure started.

In such examples, surgery planning module 718 may determine the estimated amount of OR time for the surgical procedure as a time in the time period corresponding to the output neuron that generated the highest confidence value. For instance, if the output neuron for the OR time of 30-59 minutes generated the highest confidence value, surgery planning module 718 may determine that the estimated amount of OR time for the surgical procedure is between 30 and 59 minutes.

In examples where the neural network has output neurons that correspond to different time periods, surgery planning module 718 may train the neural network using training data that comprises training data pairs. Each training data pair corresponds to a different performance of the surgical procedure. Each training data pair includes an input vector and a target value. The input vector of a training data pair may include values for each input neuron of the neural network. The target value of a training data pair may specify a time period in which the surgical procedure corresponding to the training data pair was completed. For instance, the target value of the training data pair may specify that the surgical procedure was completed within a time period from 30 to 59 minutes after the start of the surgical procedure (e.g., after the start of the OR being used for the surgical procedure).

To train the neural network using the training data, surgery planning module 718 may perform a forward pass through the neural network using the input vector of a training data pair. Surgery planning module 718 may then compare the values generated by the output neurons to the target value to determine error values. Surgery planning module 718 may then use a backpropagation algorithm to modify weights of neurons of the neural network based on the error values. Surgery planning module 718 may repeat this process for different training data pairs in the training data. In some examples, surgery planning module 718 may receive new training data pairs and may continue training the neural network as more surgical procedures are completed.

In some examples, surgery planning module 718 may estimate the amount of OR time for the surgical procedure using a plurality of machine-learned models 720, such as a plurality of neural networks. In some examples, surgery planning module 718 may generate and store data indicating a surgical plan for the surgical procedure. The surgical plan for the surgical procedure may specify the steps of the surgical procedure. In some examples, the surgical plan for the surgical procedure may further specify surgical items that are associated with specific steps of the surgical procedure.

In examples where surgery planning module 718 estimates the amount of OR time for the surgical procedure using a plurality of machine-learned models 720, the machine-learned models 720 generate output data indicating estimated amounts of time that will be required to perform separate steps of the surgical procedure. For example, a first machine-learned model may generate output data indicating an estimated amount of time to perform a first step of the surgical procedure, a second machine-learned model may generate output data indicating an estimated amount of time to perform a second step of the surgical procedure, and so on. Surgery planning module 718 may then estimate the amount of OR time for the surgical procedure based on a sum of the estimated amounts of time that will be required to perform the steps of the surgical procedure. In some examples, the estimated amount of OR time for the surgical procedure may be equal to the sum of the estimated amounts of time that will be required to perform the steps of the surgical procedure. In some examples, the estimated amount of OR time for the surgical procedure may be equal the sum of the estimated amounts of time that will be required to perform the steps of the surgical procedure plus some amount of time associated with starting and concluding the surgical procedure and/or transitioning between steps of the surgical procedure.

In some examples where surgery planning module 718 estimates the amount of OR time for the surgical procedure using a plurality of machine-learned models 720, a machine-learned model may directly output a value indicating the estimated amount of time to perform a step of the surgical procedure. For instance, at least one of the machine-learned models may be implemented as a neural network having an output neuron that generates a value indicating the estimated amount of time to perform a step of the surgical procedure. Thus, such neural networks may be similar to the neural network described in the example provided above where a single neural network is used to estimate the amount of OR time for the whole surgical procedure.

In other examples, one or more of the machine-learned models may be implemented as neural networks that have output neurons corresponding to different time periods. Thus, such neural networks may be similar to the neural network described in the example provided above where a single neural network has output neurons corresponding to different time periods and is used to estimate the amount of OR time for the whole surgical procedure. In this example, the time periods for output neurons of a neural network corresponding to an individual step of the surgical procedure may have intervals significantly shorter than the time periods used for estimating an amount of OR time for the whole surgical procedure. For instance, a first output neuron of a neural network corresponding to a specific step of the surgical procedure may correspond to 0 to 4 minutes, a second output neuron of the neural network may correspond to 5 to 9 minutes, and so on. In such examples, an output neuron of the neural network may output a confidence value that indicates a level of confidence that the step of the surgical procedure will be completed within the time period corresponding to the output neuron. Surgery planning module 718 may select the time period having the highest confidence as the estimated time amount of time required to complete the step of the surgical procedure.

In some examples, information describing the steps of the surgical procedure and the surgical items associated with the steps of the surgical procedure are presented to one or more users during the intraoperative phase of the surgical procedure. For instance, a surgeon may wear MR visualization device 213 during the surgical procedure and MR visualization device 213 may generate an MR visualization that includes virtual objects that indicate the steps of the surgical procedure and surgical items associated with specific steps of the surgical procedure. Presenting information describing the steps of the surgical procedure and the surgical items associated with the steps of the surgical procedure during the intraoperative phase of the surgical procedure may help to remind the surgeon and OR staff how they planned to perform the surgical procedure during performance of the surgical procedure. In some examples, the presented information may include checklists indicating what actions need to be performed in order to complete each step of the surgical procedure.

In some examples, surgery planning module 718 may automatically determine when a step of the surgical procedure is complete. For instance, surgery planning module 718 may automatically determine that a step of the surgical procedure is complete when a surgeon removes a surgical item associated with a next step of the surgical procedure from a storage location. In other examples, surgery planning module 718 may receive indications of user input, such as voice commands, touch input, button-push input, or other types of input to indicate the completion of steps of a surgical procedure. For instance, surgery planning module 718 may implement techniques as described in Patent Cooperation Treaty (PCT) application PCT/US2019/036978, filed Jun. 13, 2019 (the entire content of which is incorporated by reference), which describes example processes for presenting virtual checklist items in an extended reality (XR) visualization device and example ways of marking steps of surgical procedures as complete.

Based on a determination that a step of the surgical procedure is complete, surgery planning module 718 may record an amount of time that was used to complete the step of the surgical procedure. Surgery planning module 718 may then generate a new training data pair. The input vector of the training data pair may include an applicable value for the surgical procedure (e.g., anatomic parameter data, patient characteristic data, surgical parameter data, surgeon experience level, etc.). In an example where a neural network corresponding to the step of the surgical procedure has an output neuron that generates output indicating the amount of estimated amount of time required to perform the step of the surgical procedure, the target value of the training data pair indicates an amount of time it took to complete the step of the surgical procedure. In an example where a neural network corresponding to the step of the surgical procedure has output neurons corresponding to different time periods, the target value of the training data pair may indicate the time period in which the step of the surgical procedure was completed. After generating the new training data pair, surgery planning module 718 may use the new training data pair to continue the training of the neural network. In this way, the neural network may continue to improve as the step of the surgical procedure is performed more times.

As indicated above, in some examples, surgery planning module 718 may estimate an updated amount of OR time during the intraoperative phase of the surgical procedure. In some examples, when surgery planning module 718 estimates the updated amount of OR time during the intraoperative phase of the surgical procedure, surgery planning module 718 may determine the updated estimated amount of OR time in the same way that surgery planning module 718 estimates the amount of OR time during the preoperative phase, albeit with updated input data. For instance, in some examples, surgery planning module 718 may use a single machine-learned model to estimate the amount of OR time. In other examples, surgery planning module 718 may use separate machine-learned models for different steps of the surgical procedure. In such examples, surgery planning module 718 may estimate the amount of OR time based on a sum of the amount of time elapsed so far during the surgical procedure and estimates of amounts of time to perform any unfinished steps of the surgical procedure.

In examples where surgery planning module 718 estimates the updated amount of OR time for the surgical procedure during the intraoperative phase, surgery planning module 718 may estimate the updated amount of OR time in response to various events. For instance, surgery planning module 718 may estimate the updated amount of OR time for the surgical procedure in response to receiving input indicating different anatomic parameter data than the anatomic parameter data obtained during the preoperative phase of the surgical procedure. For instance, surgery planning module 718 may estimate the updated amount of OR time for the surgical procedure in response to receiving input indicating the presence of additional osteophytes that were not accounted for in the preoperative phase.

In another example, surgery planning module 718 may estimate the updated amount of OR time for the surgical procedure in response to receiving input indicating different surgical parameter data than the surgical parameter data obtained during the preoperative phase of the surgical procedure. For instance, surgery planning module 718 may estimate the updated amount of OR time for the surgical procedure in response to receiving input indicating that a surgeon has chosen a different surgical option during the surgical procedure than was selected during the preoperative phase of the surgical procedure. For example, surgery planning module 718 may receive input indicating that the surgeon has chosen to use a different type of orthopedic prosthesis than the surgeon selected during the preoperative phase of the surgical procedure.

In some examples, surgery planning module 718 may determine, during the intraoperative phase of the surgical procedure, whether different steps of the surgical procedure will need to be performed based on updated anatomic parameter data and/or updated procedure data received during the intraoperative phase of the surgical procedure. For instance, in one example involving a shoulder arthroplasty, if one or more anatomic parameters are different from what was expected (e.g., erosion of glenoid was deeper than expected), surgeon may need to perform more or fewer steps during the surgical procedure (e.g., performing a bone graft). In another example involving a shoulder surgery, if the original plan for the surgical procedure was to implant a stemless humeral implant and the surgeon selected a stemmed humeral implant instead, the surgeon may need to perform additional steps, such as sounding and compacting spongy bone tissue in the patient's humerus.

In some examples, surgery planning module 718 may determine a modified surgical plan for the surgical procedure in accordance with one or more of the examples described in PCT application no. PCT/US2019/036993, filed Jun. 13, 2019. PCT application no. PCT/US2019/036993 describes obtaining an information model specifying a first surgical plan for an orthopedic surgery to be performed on a patient; modifying the first surgical plan during an intraoperative phase of the orthopedic surgery to generate a second surgical plan; and, during the intraoperative phase of the orthopedic surgery, presenting, with a visualization device, a visualization for display that is based on the second surgical plan.

In examples where surgery planning module 718 determines the estimated amount of OR time for the surgical procedure based on a sum of estimated amounts of times to perform steps of the surgical procedure, surgery planning module 718 may estimate the amounts of time for remaining steps of the surgical procedure according to the modified surgical plan. For instance, in some such examples, machine-learned models 720 may include a machine-learned model (e.g., a neural network) for each potential step in a type of surgical procedure. Surgery planning module 718 may determine an estimated time to complete a step based on output of the machine-learned model for the step. In such examples, when surgery planning module 718 determines the estimated amount of OR time for the surgical procedure during the intraoperative phase of the orthopedic procedure, surgery planning module 718 may use the machine-learned models corresponding to remaining steps of the surgical procedure as specified by an original or modified surgical plan for the surgical procedure. Surgery planning module 718 may estimate the amount of remaining OR time for the surgical procedure based on a sum of the estimated times to complete each of the remaining steps of the surgical procedure. In some examples, during the intraoperative phase of the surgical procedure, surgery planning module 718 may estimate the amount of OR time for the surgical procedure based on a sum of the amount of time elapsed so far during the surgical procedure and the estimated amounts of time required to complete each of the remaining steps of the surgical procedure.

In examples where surgery planning module 718 estimates the amount of OR time for the surgical procedure using a plurality of machine-learned models 720, different machine-learned models in the plurality of machine-learned models 720 may have different inputs. For instance, in an example where surgery planning module 718 uses different neural networks to estimate amounts of time to perform different steps of the surgical procedure, a first neural network that estimates an amount of time to perform a first step of the surgical procedure may have input neurons that accept a different set of input from input neurons of a second neural network that estimates an amount of time to perform a second step of the surgical procedure. For instance, in one example, a first neural network may estimate an amount of time to perform a step of reaming a patient's glenoid and a second neural network may estimate an amount of time to perform a step of implanting a humeral prosthesis in the patient's humerus. In this example, the surgical parameter data may include data indicating a type of reaming bit and data indicating a type of humeral prosthesis. In this example, it may be unnecessary to provide the data indicating the type of humeral prosthesis to the first neural network and it may be unnecessary to provide the data indicating the type of reaming bit to the second neural network.

Furthermore, in the example of FIG. 19, surgery planning module 718 may output an indication of the estimated amount of OR time for the surgical procedure (1908). Surgery planning module 718 may output the indication of the estimated amount of OR time for the surgical procedure in any of a variety of ways. For instance, in one example, the MR visualization device 213 (FIG. 2) may output an MR visualization that contains text or graphical data indicating the estimated amount of OR time for the surgical procedure. In some examples, another type of display device (e.g., one of display devices 708 (FIG. 7)) or output device (e.g., one of output devices 712 (FIG. 7)) may output text, graphical data, or audible data indicating the estimated amount of OR time for the surgical procedure.

As indicated above, in some examples, surgery planning module 718 may estimate an updated amount of OR time during the intraoperative phase of the surgical procedure. In such examples, surgery planning module 718 may output the indication of the estimated amount of OR time for the surgical procedure during the intraoperative phase of the surgical procedure. In some examples where surgery planning module 718 outputs the indication of the estimated amount of OR time for the surgical procedure during the intraoperative phase of the surgical procedure, surgery planning module 718 may output the indication of the estimated amount of OR time for the surgical procedure to the surgeon or other persons in the OR.

In some examples where surgery planning module 718 outputs the indication of the estimated amount of OR time for the surgical procedure during the intraoperative phase of the surgical procedure, surgery planning module 718 may output the indication of the estimated amount of OR time for the surgical procedure to users outside the OR, such as hospital scheduling staff. Thus, if the anatomic parameters or surgical parameters change during the surgical procedure and surgery planning module 718 determines that the surgical procedure will run long, the hospital scheduling staff may cancel or reschedule one or more surgical procedures due to occur in the OR after completion of the surgical procedure on the current patient. Conversely, if the anatomic parameters or surgical parameters change during the surgical procedure and surgery planning module 718 determines that the surgical procedure will run short (e.g., because the surgeon determines that specific steps of the surgical procedure are unnecessary or cannot be performed), the hospital scheduling staff may add one or more surgical procedures to a schedule for the OR or move forward one or more surgical procedures scheduled for the OR. Advantageously, this may allow automatic updates regarding the amount of time the OR is expected to be in use without anyone outside the OR having to ask anyone inside the OR about the amount of time the OR is expected to be in use. This may reduce distraction and time pressure experienced by the surgeon, which may lead to better surgical outcomes.

In the example of FIG. 19, virtual planning system 701 may determine an OR schedule for an OR based at least in part on the estimated amount of OR time for the surgical procedure (1910). In some examples, a computing system separate from virtual planning system 701 determines the OR schedule. However, for ease of explanation, this disclosure assumes that virtual planning system 701 determines the OR schedule.

For instance, in one example, virtual planning system 701 may scan through a schedule for the OR chronologically and identify a first available unallocated time slot that has a duration longer than the estimated amount of OR time for the surgical procedure. An unallocated time slot is a time slot in which the OR has not been allocated for use.

In some examples where surgery planning module 718 generates confidence values for a plurality of time periods, the estimated amount of OR time for the surgical procedure may be the time period with the greatest confidence value. However, rather than using the first available unallocated time slot that virtual planning system 701 identifies that has a duration longer than the estimated amount of OR time for the surgical procedure, surgery planning module 718 may determine a cut-off time period. The cut-off time period is a time period immediately preceding a first-occurring time period that is longer than the time period having the greatest confidence value and that has a confidence value below a threshold. The threshold may be configurable (e.g., by hospital scheduling staff or other parties). Virtual planning system 701 may then determine the OR schedule using the cut-off time duration instead of the time duration having the greatest confidence value. In this way, surgery planning system 701 may build time into the OR schedule for possible time overruns during the surgical procedure.

As in the previous example, the estimated amount of OR time for the surgical procedure may be the time duration with the greatest confidence value. However, in some examples, surgery planning system 701 may analyze a distribution of the confidence values and determine the OR schedule based on the distribution. For instance, surgery planning system 701 may determine that the distribution of confidence values is biased toward smaller time durations than the time duration with the greatest confidence value. Accordingly, surgery planning system 701 may build in a smaller amount of time after the time duration with the greatest confidence value. For instance, if the time durations are in 30 minute increments and the two time durations before the time duration with the highest confidence value have confidence values almost as high as the highest confidence value while the two time durations after the time duration with the highest confidence value are significantly lower than the highest confidence value, surgery planning system 701 may identify an unallocated time slot that is only 30 minutes longer than the estimated amount of OR time. In contrast, in this example, if the two time durations after the time duration with the highest confidence value is almost as high as the highest confidence value, surgery planning system 701 may identify an unallocated time slot that is 60 minutes longer than the time duration having the highest confidence value.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuity,” as used herein may refer to any of the foregoing structures or any other structure suitable for implementation of the techniques described herein.

Various examples have been described. These and other examples are within the scope of the following claims.

1. A computer-implemented method comprising:
obtaining, by a computing system, patient characteristics of a patient;
obtaining, by the computing system, prosthetic implant characteristics of a prosthetic implant;
determining, by the computing system, information indicative of an operational duration of the prosthetic implant based on the patient characteristics and the prosthetic implant characteristics, wherein determining the information indicative of the operational duration of the prosthetic implant comprises:
receiving, with a machine-learned model of the computing system, the patient characteristics and the prosthetic implant characteristics;
applying, with the computing system, model parameters of the machine-learned model to the patient characteristics and the prosthetic implant characteristics, wherein the model parameters of the machine-learned model are generated based on a machine learning dataset, wherein the machine learning dataset includes one or more of pre-operative scans of a set of patients, patient characteristics of the set of patients, information indicative of previous surgical plans using different prosthetic implants, and information indicative of surgical results; and
determining the information indicative of the operational duration of the prosthetic implant from the application of the model parameters of the machine-learned model; and
outputting, by the computing system, the information indicative of the operational duration of the prosthetic implant.

2. The method of claim 1, wherein determining information indicative of the operational duration of the prosthetic implant comprises determining information indicative of a likelihood that the prosthetic implant will serve a function of the prosthetic implant for a certain amount of time.

3-4. (canceled)

5. The method of claim 1, wherein the patient characteristics include one or more age of the patient, gender of the patient, diseases of the patient, whether the patient is a smoker, or fatty infiltration at target bone where the prosthetic implant is to be implanted.

6. The method of claim 1, wherein the prosthetic implant characteristics of the prosthetic implant comprise one or more of a type of prosthetic implant or dimensions of the prosthetic implant.

7. The method of claim 1, wherein determining the information indicative of the operational duration of the prosthetic implant comprises determining information indicative of the operational duration of the prosthetic implant for a first surgical procedure, the method further comprising:
determining information indicative of a plurality of operational durations for the prosthetic implant for a plurality of surgical procedures.

8. The method of claim 1, wherein the prosthetic implant comprises a first prosthetic implant, the method further comprising:
obtaining prosthetic implant characteristics of a plurality of prosthetic implants, wherein the plurality of prosthetic implants includes the first prosthetic implant;
determining information indicative of the operational duration of each of the plurality of prosthetic implants based on the patient characteristics and respective prosthetic implant characteristics of the plurality of prosthetic implants; and
outputting the information indicative of the respective operational duration of each of the plurality of prosthetic implants.

9. The method of claim 1, wherein the prosthetic implant comprises a first prosthetic implant, the method further comprising:
obtaining prosthetic implant characteristics of a plurality of prosthetic implants, wherein the plurality of prosthetic implants includes the first prosthetic implant;
determining information indicative of the operational duration of each of the plurality of prosthetic implants based on the patient characteristics and respective prosthetic implant characteristics of the plurality of prosthetic implants;
comparing the information indicative of the operational duration of each of the plurality of prosthetic implants, including the information indicative of the operational duration of the first prosthetic implant, with each other;
selecting one of the plurality of prosthetic implants based on the comparison of the information indicative of the operational duration of each of the plurality of prosthetic implants; and
outputting information indicating that the selected prosthetic implant is a recommended prosthetic implant,
wherein outputting the information indicative of the operational duration of the prosthetic implant comprises outputting information indicative of the operational duration of the first prosthetic implant responsive to the first prosthetic implant being the selected one of the plurality of prosthetic implants.

10. The method of claim 9, further comprising:
determining one or more feasibility scores for one or more of the plurality of prosthetic implants;
comparing the one or more feasibility scores,
wherein selecting one of the plurality of prosthetic implants comprises selecting one of the plurality of prosthetic implants based on the comparison of the information indicative of the operational duration of each of the plurality of prosthetic implants and the comparison of the one or more feasibility scores.

11. A computing system comprising:
memory configured to store patient characteristics of a patient and prosthetic implant characteristics of a prosthetic implant; and
one or more processors, coupled to the memory, and configured to
obtain the patient characteristics of the patient;
obtain the prosthetic implant characteristics of the prosthetic implant;
determine information indicative of an operational duration of the prosthetic implant based on the patient characteristics and the prosthetic implant characteristics,. wherein to determine the information indicative of the operational duration of the prosthetic implant, the one or more processors are configured to:
receive, with a machine-learned model of the computing system, the patient characteristics and the prosthetic implant characteristics;
apply model parameters of the machine-learned model to the patient characteristics and the prosthetic implant characteristics, wherein the model parameters of the machine-learned model are generated based on a machine learning dataset, wherein the machine learning dataset includes one or more of pre-operative scans of a set of patients, patient characteristics of the set of patients, information indicative of previous surgical plans using different prosthetic implants, and information indicative of surgical results; and
determine the information indicative of the operational duration of the prosthetic implant from the application of the model parameters of the machine-learned model; and
output the information indicative of the operational duration of the prosthetic implant.

12. The system of claim 11, wherein to determine information indicative of the operational duration of the prosthetic implant, the one or more processors are configured to determine information indicative of a likelihood that the prosthetic implant will serve a function of the prosthetic implant for a certain amount of time.

13-14. (canceled)

15. The system of claim 11, wherein the patient characteristics include one or more age of the patient, gender of the patient, diseases of the patient, whether the patient is a smoker, or fatty infiltration at target bone where the prosthetic implant is to be implanted.

16. The system of claim 11, wherein the prosthetic implant characteristics of the prosthetic implant comprise one or more of a type of prosthetic implant or dimensions of the prosthetic implant.

17. The system of claim 11, wherein to determine the information indicative of the operational duration of the prosthetic implant, the one or more processors are configured to determine information indicative of the operational duration of the prosthetic implant for a first surgical procedure, wherein the one or more processors are configured to:
determine information indicative of a plurality of operational durations for the prosthetic implant for a plurality of surgical procedures.

18. The system of claim 11, wherein the prosthetic implant comprises a first prosthetic implant, the one or more processors are configured to:
obtain prosthetic implant characteristics of a plurality of prosthetic implants, wherein the plurality of prosthetic implants includes the first prosthetic implant;
determine information indicative of the operational duration of each of the plurality of prosthetic implants based on the patient characteristics and respective prosthetic implant characteristics of the plurality of prosthetic implants; and
output the information indicative of the respective operational duration of each of the plurality of prosthetic implants.

19. The system of claim 11, wherein the prosthetic implant comprises a first prosthetic implant, and wherein the one or more processors are configured to:
obtain prosthetic implant characteristics of a plurality of prosthetic implants, wherein the plurality of prosthetic implants includes the first prosthetic implant;
determine information indicative of the operational duration of each of the plurality of prosthetic implants based on the patient characteristics and respective prosthetic implant characteristics of the plurality of prosthetic implants;
compare the information indicative of the operational duration of each of the plurality of prosthetic implants, including the information indicative of the operational duration of the first prosthetic implant, with each other;
select one of the plurality of prosthetic implants based on the comparison of the information indicative of the operational duration of each of the plurality of prosthetic implants; and
output information indicating that the selected prosthetic implant is a recommended prosthetic implant,
wherein to output the information indicative of the operational duration of the prosthetic implant, the one or more processors are configured to output information indicative of the operational duration of the first prosthetic implant responsive to the first prosthetic implant being the selected one of the plurality of prosthetic implants.

20. The system of claim 19, wherein the one or more processors are configured to:
determine one or more feasibility scores for one or more of the plurality of prosthetic implants;
compare the one or more feasibility scores,
wherein to select one of the plurality of prosthetic implants, the one or more processors are configured to select one of the plurality of prosthetic implants based on the comparison of the information indicative of the operational duration of each of the plurality of prosthetic implants and the comparison of the one or more feasibility scores.

21-52. (canceled)

53. A non-transitory computer-readable storage medium storing instructions thereon that when executed cause one or more processors to:
obtain patient characteristics of a patient;
obtain prosthetic implant characteristics of a prosthetic implant;
determine information indicative of an operational duration of the prosthetic implant based on the patient characteristics and the prosthetic implant characteristics, wherein the instructions that cause the one or more processors to determine the information indicative of the operational duration of the prosthetic implant comprise instructions that cause the one or more processors to:
receive, with a machine-learned model, the patient characteristics and the prosthetic implant characteristics;
apply model parameters of the machine-learned model to the patient characteristics and the prosthetic implant characteristics, wherein the model parameters of the machine-learned model are generated based on a machine learning dataset, wherein the machine learning dataset includes one or more of pre-operative scans of a set of patients, patient characteristics of the set of patients, information indicative of previous surgical plans using different prosthetic implants, and information indicative of surgical results; and
determine the information indicative of the operational duration of the prosthetic implant from the application of the model parameters of the machine-learned model; and
output the information indicative of the operational duration of the prosthetic implant.

54. The non-transitory computer-readable storage medium of claim 53, wherein the instructions that cause the one or more processors to determine information indicative of the operational duration of the prosthetic implant comprise instructions that cause the one or more processors to determine information indicative of a likelihood that the prosthetic implant will serve a function of the prosthetic implant for a certain amount of time.

55. The non-transitory computer-readable storage medium of claim 53, wherein the prosthetic implant comprises a first prosthetic implant, and wherein the instructions further comprise instructions that cause the one or more processors to:
obtain prosthetic implant characteristics of a plurality of prosthetic implants, wherein the plurality of prosthetic implants includes the first prosthetic implant;
determine information indicative of the operational duration of each of the plurality of prosthetic implants based on the patient characteristics and respective prosthetic implant characteristics of the plurality of prosthetic implants; and
output the information indicative of the respective operational duration of each of the plurality of prosthetic implants.

56. The non-transitory computer-readable storage medium of claim 53, wherein the prosthetic implant comprises a first prosthetic implant, and wherein the instructions further comprise instructions that cause the one or more processors to:
obtain prosthetic implant characteristics of a plurality of prosthetic implants, wherein the plurality of prosthetic implants includes the first prosthetic implant;
determine information indicative of the operational duration of each of the plurality of prosthetic implants based on the patient characteristics and respective prosthetic implant characteristics of the plurality of prosthetic implants;
compare the information indicative of the operational duration of each of the plurality of prosthetic implants, including the information indicative of the operational duration of the first prosthetic implant, with each other;
select one of the plurality of prosthetic implants based on the comparison of the information indicative of the operational duration of each of the plurality of prosthetic implants; and
output information indicating that the selected prosthetic implant is a recommended prosthetic implant,
wherein the instructions that cause the one or more processors to output the information indicative of the operational duration of the prosthetic implant comprise instructions that cause the one or more processors to output information indicative of the operational duration of the first prosthetic implant responsive to the first prosthetic implant being the selected one of the plurality of prosthetic implants.
